SEQUENCE LISTING (198 sequences)

SEQ ID NO: 1 - protein, 8 aa, synthetic construct
GFSLTSYG

SEQ ID NO: 2 - protein, 8 aa, synthetic construct
GYTFTSYW

SEQ ID NO: 3 - protein, 8 aa, synthetic construct
GSTFNRYW

SEQ ID NO: 4 - protein, 8 aa, synthetic construct
GFTFTDYN

SEQ ID NO: 5 - protein, 8 aa, synthetic construct
GYSFTAYT

SEQ ID NO: 6 - protein, 8 aa, synthetic construct
GFNIKDDY

SEQ ID NO: 7 - protein, 8 aa, synthetic construct
GYTFTSHW

SEQ ID NO: 8 - protein, 8 aa, synthetic construct
GFTFSTFA

SEQ ID NO: 9 - protein, 8 aa, synthetic construct
GFTFTDHT

SEQ ID NO: 10 - protein, 8 aa, synthetic construct
GFSLTSYD

SEQ ID NO: 11 - protein, 8 aa, synthetic construct
GYTFTAYW

SEQ ID NO: 12 - protein, 7 aa, synthetic construct
MWGGGST

SEQ ID NO: 13 - protein, 8 aa, synthetic construct
IYPGRGST

SEQ ID NO: 14 - protein, 8 aa, synthetic construct
ILPGSGNT

SEQ ID NO: 15 - protein, 8 aa, synthetic construct
INPNNGRS

SEQ ID NO: 16 - protein, 8 aa, synthetic construct
INPYNGYA

SEQ ID NO: 17 - protein, 8 aa, synthetic construct
IDPENGDT

SEQ ID NO: 18 - protein, 8 aa, synthetic construct
IYPGSGNT

SEQ ID NO: 19 - protein, 9 aa, synthetic construct
ITGDGGTYT

SEQ ID NO: 20 - protein, 8 aa, synthetic construct
IYPRDGYT

SEQ ID NO: 21 - protein, 8 aa, synthetic construct
IWGGGGST

SEQ ID NO: 22 - protein, 7 aa, synthetic construct
IWTGGGT

SEQ ID NO: 23 - protein, 8 aa, synthetic construct
IDPNSGYT

SEQ ID NO: 24 - protein, 15 aa, synthetic construct
ARRTYSDSYYYEMDY

SEQ ID NO: 25 - protein, 9 aa, synthetic construct
ARELGGFAY

SEQ ID NO: 26 - protein, 14 aa, synthetic construct
ARLDYYGSRYYFDY

SEQ ID NO: 27 - protein, 13 aa, synthetic construct
AREDWEGFYAMDY

SEQ ID NO: 28 - protein, 9 aa, synthetic construct
ARVGYYFDY

SEQ ID NO: 29 - protein, 6 aa, synthetic construct
TGYFDY

SEQ ID NO: 30 - protein, 9 aa, synthetic construct
ARERGGFDY

SEQ ID NO: 31 - protein, 11 aa, synthetic construct
ARHSVSSWFAY

SEQ ID NO: 32 - protein, 11 aa, synthetic construct
ARPTSLLRFPY

SEQ ID NO: 33 - protein, 14 aa, synthetic construct
ARHNYDGYYYSLDY

SEQ ID NO: 34 - protein, 14 aa, synthetic construct
ARHNYDNYYYTLDY

SEQ ID NO: 35 - protein, 11 aa, synthetic construct
VRDLFPYAMDY

SEQ ID NO: 36 - protein, 10 aa, synthetic construct
ARGHFGYDDS

SEQ ID NO: 37 - protein, 7 aa, synthetic construct
SSVSSRY

SEQ ID NO: 38 - protein, 7 aa, synthetic construct
SYVSSSY

SEQ ID NO: 39 - protein, 6 aa, synthetic construct
QDIRNY

SEQ ID NO: 40 - protein, 6 aa, synthetic construct
QNVDTD

SEQ ID NO: 41 - protein, 5 aa, synthetic construct
SSVSY

SEQ ID NO: 42 - protein, 6 aa, synthetic construct
QNINLW

SEQ ID NO: 43 - protein, 6 aa, synthetic construct
QDISNF

SEQ ID NO: 44 - protein, 5 aa, synthetic construct
SSVNY

SEQ ID NO: 45 - protein, 6 aa, synthetic construct
EDIDSY

SEQ ID NO: 46 - protein, 6 aa, synthetic construct
QTIGTS

SEQ ID NO: 47 - protein, 5 aa, synthetic construct
SRVSY

SEQ ID NO: 48-55 - skipped (8 entries)

SEQ ID NO: 56 - protein, 9 aa, synthetic construct
QQYHSDPLT

SEQ ID NO: 57 - protein, 9 aa, synthetic construct
HQYHLSPWT

SEQ ID NO: 58 - protein, 9 aa, synthetic construct
QQGDPLPPT

SEQ ID NO: 59 - protein, 9 aa, synthetic construct
EQYSSYPYT

SEQ ID NO: 60 - protein, 9 aa, synthetic construct
QQFTSSPYT

SEQ ID NO: 61 - protein, 9 aa, synthetic construct
LQGQSYPWT

SEQ ID NO: 62 - protein, 9 aa, synthetic construct
QQDNNHPYT

SEQ ID NO: 63 - protein, 9 aa, synthetic construct
QQFTSSPWT

SEQ ID NO: 64 - protein, 9 aa, synthetic construct
QHYYSIPYT

SEQ ID NO: 65 - protein, 9 aa, synthetic construct
QQYHGDPLT

SEQ ID NO: 66 - protein, 9 aa, synthetic construct
QQTNSWPLT

SEQ ID NO: 67 - protein, 9 aa, synthetic construct
QQRSSYPLT

SEQ ID NO: 68 - protein, 166 aa, synthetic construct
  VARIANT 26..50: note = Any amino acid
  VARIANT 27..50: note = Residues may be deleted
  VARIANT 68..92: note = Any amino acid
  VARIANT 69..92: note = Residues may be deleted
  VARIANT 131..155: note = Any amino acid
  VARIANT 132..155: note = Residues may be deleted
QVQLVQSGAEVKKPGASVKVSCKASXXXXXXXXXXXXXXXXXXXXXXXXXISWVRQAPGQGLEWMGWXXXXXXXXXXXXXXXXXXXXXXXXXNYAQKLQGRVTMTTDTSTSTAYMELRSLRSDDTAVYYCXXXXXXXXXXXXXXXXXXXXXXXXXWGQGTLVTVSS

SEQ ID NO: 69 - protein, 166 aa, synthetic construct
  VARIANT 26..50: note = Any amino acid
  VARIANT 27..50: note = Residues may be deleted
  VARIANT 68..92: note = Any amino acid
  VARIANT 69..92: note = Residues may be deleted
  VARIANT 131..155: note = Any amino acid
  VARIANT 132..155: note = Residues may be deleted
QVQLVQSGAEVKKPGASVKVSCKASXXXXXXXXXXXXXXXXXXXXXXXXXMHWVRQAPGQGLEWMGRXXXXXXXXXXXXXXXXXXXXXXXXXNYAQKFQGRVTSTRDTSISTAYMELSRLRSDDTVVYYCXXXXXXXXXXXXXXXXXXXXXXXXXWGQGTLVTVSS

SEQ ID NO: 70 - protein, 166 aa, synthetic construct
  VARIANT 26..50: note = Any amino acid
  VARIANT 27..50: note = Residues may be deleted
  VARIANT 68..92: note = Any amino acid
  VARIANT 69..92: note = Residues may be deleted
  VARIANT 131..155: note = Any amino acid
  VARIANT 132..155: note = Residues may be deleted
QVQLVQSGAEVKKPGASVKVSCKASXXXXXXXXXXXXXXXXXXXXXXXXXMHWVRQAPGQRLEWMGWXXXXXXXXXXXXXXXXXXXXXXXXXKYSQKFQGRVTITRDTSASTAYMELSSLRSEDTAVYYCXXXXXXXXXXXXXXXXXXXXXXXXXWGQGTLVTVSS

SEQ ID NO: 71 - protein, 166 aa, synthetic construct
  VARIANT 26..50: note = Any amino acid
  VARIANT 27..50: note = Residues may be deleted
  VARIANT 68..92: note = Any amino acid
  VARIANT 69..92: note = Residues may be deleted
  VARIANT 131..155: note = Any amino acid
  VARIANT 132..155: note = Residues may be deleted
QVQLVQSGAEVKKPGASVKVSCKASXXXXXXXXXXXXXXXXXXXXXXXXXISWVRQAPGQGLEWMGDXXXXXXXXXXXXXXXXXXXXXXXXXNYNEKLQGRVTMTVDTSTSTAYMELRSLRSDDTAVYYCXXXXXXXXXXXXXXXXXXXXXXXXXWGQGTLVTVSS

SEQ ID NO: 72 - protein, 166 aa, synthetic construct
  VARIANT 26..50: note = Any amino acid
  VARIANT 27..50: note = Residues may be deleted
  VARIANT 68..92: note = Any amino acid
  VARIANT 69..92: note = Residues may be deleted
  VARIANT 131..155: note = Any amino acid
  VARIANT 132..155: note = Residues may be deleted
QVQLVQSGAEVKKPGASVKVSCKASXXXXXXXXXXXXXXXXXXXXXXXXXITWVRQAPGQGLEWMGDXXXXXXXXXXXXXXXXXXXXXXXXXNYNEKFQGRVTLTVDTSISTAYMELSRLRSDDTVVYYCXXXXXXXXXXXXXXXXXXXXXXXXXWGQGTLVTVSS

SEQ ID NO: 73 - protein, 166 aa, synthetic construct
  VARIANT 26..50: note = Any amino acid
  VARIANT 27..50: note = Residues may be deleted
  VARIANT 68..92: note = Any amino acid
  VARIANT 69..92: note = Residues may be deleted
  VARIANT 131..155: note = Any amino acid
  VARIANT 132..155: note = Residues may be deleted
QVQLVQSGAEVKKPGASVKVSCKASXXXXXXXXXXXXXXXXXXXXXXXXXITWVRQAPGQRLEWMGDXXXXXXXXXXXXXXXXXXXXXXXXXNYNEKFQGRVTITVDTSASTAYMELSSLRSEDTAVYYCXXXXXXXXXXXXXXXXXXXXXXXXXWGQGTLVTVSS

SEQ ID NO: 74 - protein, 166 aa, synthetic construct
  VARIANT 26..50: note = Any amino acid
  VARIANT 27..50: note = Residues may be deleted
  VARIANT 68..92: note = Any amino acid
  VARIANT 69..92: note = Residues may be deleted
  VARIANT 131..155: note = Any amino acid
  VARIANT 132..155: note = Residues may be deleted
QVQLQQPGAELVKPGASVKMSCKASXXXXXXXXXXXXXXXXXXXXXXXXXITWVKQRPGQGLEWIGDXXXXXXXXXXXXXXXXXXXXXXXXXNYNEKFKSKATLTVDTSSSTAYMQLSSLTSEDSADYYCXXXXXXXXXXXXXXXXXXXXXXXXXWGQGTTLTVSS

SEQ ID NO: 75 - protein, 164 aa, synthetic construct
  VARIANT 27..51: note = Any amino acid
  VARIANT 28..51: note = Residues may be deleted
  VARIANT 69..93: note = Any amino acid
  VARIANT 70..93: note = Residues may be deleted
  VARIANT 130..154: note = Any amino acid
  VARIANT 131..154: note = Residues may be deleted
DIQMTQSPSSLSASVGDRVTITCRASXXXXXXXXXXXXXXXXXXXXXXXXXLAWYQQKPGKVPKLLIYXXXXXXXXXXXXXXXXXXXXXXXXXTLQSGVPSRFSGSGSGTDFTLTISSLQPEDVATYYCXXXXXXXXXXXXXXXXXXXXXXXXXFGQGTKLEIK

SEQ ID NO: 76 - protein, 164 aa, synthetic construct
  VARIANT 27..51: note = Any amino acid
  VARIANT 28..51: note = Residues may be deleted
  VARIANT 69..93: note = Any amino acid
  VARIANT 70..93: note = Residues may be deleted
  VARIANT 130..154: note = Any amino acid
  VARIANT 131..154: note = Residues may be deleted
DIQMTQSPSSLSASVGDRVTITCQASXXXXXXXXXXXXXXXXXXXXXXXXXLNWYQQKPGKAPKLLIYXXXXXXXXXXXXXXXXXXXXXXXXXNLETGVPSRFSGSGSGTDFTFTISSLQPEDIATYYCXXXXXXXXXXXXXXXXXXXXXXXXXFGQGTKLEIK

SEQ ID NO: 77 - protein, 164 aa, synthetic construct
  VARIANT 27..51: note = Any amino acid
  VARIANT 28..51: note = Residues may be deleted
  VARIANT 69..93: note = Any amino acid
  VARIANT 70..93: note = Residues may be deleted
  VARIANT 130..154: note = Any amino acid
  VARIANT 131..154: note = Residues may be deleted
DIQMTQSPSSLSASVGDRVTITCRASXXXXXXXXXXXXXXXXXXXXXXXXXLNWYQQKPGKAPKLLIYXXXXXXXXXXXXXXXXXXXXXXXXXSLQSGVPSRFSGSGSGTDFTLTISSLQPEDFATYYCXXXXXXXXXXXXXXXXXXXXXXXXXFGQGTKLEIK

SEQ ID NO: 78 - protein, 164 aa, synthetic construct
  VARIANT 27..51: note = Any amino acid
  VARIANT 28..51: note = Residues may be deleted
  VARIANT 69..93: note = Any amino acid
  VARIANT 70..93: note = Residues may be deleted
  VARIANT 130..154: note = Any amino acid
  VARIANT 131..154: note = Residues may be deleted
DIQMTQSPSSLSASVGDRVTITCRASXXXXXXXXXXXXXXXXXXXXXXXXXLNWYQQKPEGTLKLLIYXXXXXXXXXXXXXXXXXXXXXXXXXGLHSGVPSRFSGSGSGTDFTLTISSLQPEDVATYYCXXXXXXXXXXXXXXXXXXXXXXXXXFGQGTKLEIK

SEQ ID NO: 79 - protein, 164 aa, synthetic construct
  VARIANT 27..51: note = Any amino acid
  VARIANT 28..51: note = Residues may be deleted
  VARIANT 69..93: note = Any amino acid
  VARIANT 70..93: note = Residues may be deleted
  VARIANT 130..154: note = Any amino acid
  VARIANT 131..154: note = Residues may be deleted
DIQMTQSPSSLSASVGDRVTITCQASXXXXXXXXXXXXXXXXXXXXXXXXXLNWYQQKPGKTLKLLIYXXXXXXXXXXXXXXXXXXXXXXXXXGLHTGVPSRFSGSGSGTDFTFTISSLQPEDIATYYCXXXXXXXXXXXXXXXXXXXXXXXXXFGQGTKLEIK

SEQ ID NO: 80 - protein, 164 aa, synthetic construct
  VARIANT 27..51: note = Any amino acid
  VARIANT 28..51: note = Residues may be deleted
  VARIANT 69..93: note = Any amino acid
  VARIANT 70..93: note = Residues may be deleted
  VARIANT 130..154: note = Any amino acid
  VARIANT 131..154: note = Residues may be deleted
DIQMTQSPSSLSASVGDRVTITCRASXXXXXXXXXXXXXXXXXXXXXXXXXLNWYQQKPGKAPKLLIYXXXXXXXXXXXXXXXXXXXXXXXXXGLHSGVPSRFSGSGSGTDFTLTISSLQPEDFATYYCXXXXXXXXXXXXXXXXXXXXXXXXXFGQGTKLEIK

SEQ ID NO: 81 - protein, 164 aa, synthetic construct
  VARIANT 27..51: note = Any amino acid
  VARIANT 28..51: note = Residues may be deleted
  VARIANT 69..93: note = Any amino acid
  VARIANT 70..93: note = Residues may be deleted
  VARIANT 130..154: note = Any amino acid
  VARIANT 131..154: note = Residues may be deleted
DIQMTQTTSSLSASLGDRVTINCRASXXXXXXXXXXXXXXXXXXXXXXXXXLNWYQQKPDGTLKLLIYXXXXXXXXXXXXXXXXXXXXXXXXXGLHSGVPSRFSGSGSGTDYSLTISNLEEEDIATYFCXXXXXXXXXXXXXXXXXXXXXXXXXFGSGTKLEIK

SEQ ID NO: 82 - protein, 121 aa, synthetic construct
QVQLKESGPGLVAPSQSLSITCTVSGFSLTSYGIDWVRQPPGKGLEWLGIMWGGGSTNYNSALMSRLSISKDISKSQVFLKVNSLQTDDTAMYYCARRTYSDSYYYEMDYWGQGTSVTVSS

SEQ ID NO: 83 - protein, 116 aa, synthetic construct
QVQLQQPGTELVKPGASVKMSCKASGYTFTSYWITWVKQRPGQGLEWIGDIYPGRGSTNYNEKFKSKATLTVDTSSSTAYMQLSSLTSEDSAVYYCARELGGFAYWGQGTLVTVSA

SEQ ID NO: 84 - protein, 121 aa, synthetic construct
QVQLQQSGAELMKPGASVKISCKATGSTFNRYWIEWVKQRPGHGLEWIGEILPGSGNTNYNEKFKGKATFTADTSSNTAYMQLSSLTSEDSAVYYCARLDYYGSRYYFDYWGQGTTLTVSS

SEQ ID NO: 85 - protein, 120 aa, synthetic construct
EVQLQQSGPELVKPGASVKMSCKASGFTFTDYNIHWVKQSHGKSLEWIGYINPNNGRSSYNLYFKDKATLTVNKSSSTAYMELRSLTSEDSAVYYCAREDWEGFYAMDYWGQGTSVTVSS

SEQ ID NO: 86 - protein, 116 aa, synthetic construct
EVQLQQSGPELVKPGASMKISCKASGYSFTAYTMNWVRQSHGKNLEWIGLINPYNGYANYNQKFKGKATLTVDKSSSTAYMDLLSLTSEDSAVYYCARVGYYFDYWGQGTTLTVSS

SEQ ID NO: 87 - protein, 113 aa, synthetic construct
EVQLQQSGAELVRPGASVKLSCTSSGFNIKDDYMHWVKQRPEQGLEWIGWIDPENGDTEYASKFQGKATITADTSSNTAYLQLNSLTSEDTAVYYCTGYFDYWGQGTTLTVSS

SEQ ID NO: 88 - protein, 116 aa, synthetic construct
QVQLQQPGAELVKPGASVKMSCKASGYTFTSHWITWVKQRPGQGLEWIGDIYPGSGNTNYNEKFKSKATLTVDTSSSTAYMQLSSLTSEDSADYYCARERGGFDYWGQGTTLTVSS

SEQ ID NO: 89 - protein, 119 aa, synthetic construct
DVILVESGGDLVKPGGSLKLSCAASGFTFSTFAMSWVRQTPEKRLEWVASITGDGGTYTYYSDSVKGRFTISRDNARNTLYLQMSSLRSEDTAFYYCARHSVSSWFAYWGQGTLVTVSA

SEQ ID NO: 90 - protein, 118 aa, synthetic construct
QVQLLQSDAELVKPGASVKISCKVSGFTFTDHTLHWMKQRPEQGLEWIGYIYPRDGYTKYNEKFKGKATLTADKSSSTAYMQLDSLTSEDSAVYFCARPTSLLRFPYWGQGTLVTVSA

SEQ ID NO: 91 - protein, 121 aa, synthetic construct
QVQLKESGPGLVAPSQSLSITCTVSGFSLTSYGVDWVRQPPGKGLEWLGVIWGGGGSTNYNSALMSRLSISKDNSKSQVFLKMNSLQTDDTAMYYCARHNYDGYYYSLDYWGQGTSVTVSS

SEQ ID NO: 92 - protein, 121 aa, synthetic construct
QVQLKESGPGLVAPSQSLSITCTVSGFSLTSYGVDWVRQPPGKGLEWLGVIWGGGGSTNYNSALMSRLSISKDNSKSQVFLKMNSLQTDDTAMYYCARHNYDNYYYTLDYWGQGTSVTVSS

SEQ ID NO: 93 - protein, 117 aa, synthetic construct
QVQLKESGPGLVAPSQSLSITCTVSGFSLTSYDISWIRQPPGKGLEWLGVIWTGGGTNYNSPFMSRLTISKDNSRSQVFLKMNSLQTDDTAMYYCVRDLFPYAMDYWGQGTSVTVSS

SEQ ID NO: 94 - protein, 117 aa, synthetic construct
QVQLQQSGAELAKPGASVKMSCKASGYTFTAYWIHWVKQRPGQGLEWIGYIDPNSGYTDYNQIFKDKATLTADRSSSTAYMQLNSLTSEDSAVYYCARGHFGYDDSWGQGITLTVSS

SEQ ID NO: 95 - protein, 108 aa, synthetic construct
QIVLTQSPAIMSASPGEKVTMTCSASSSVSSRYLHWYQQKSGASPKLWIYGTSNLASGVPARFSGSGSGTSYSLTISSVEAEDAATYYCQQYHSDPLTFGAGTKLELK

SEQ ID NO: 96 - protein, 108 aa, synthetic construct
QIVLTQSPAIMSASLGERVTMTCTASSYVSSSYLHWYQQKPGSSPTLWIYTTSNLASGVPARFSGSGSGTSYSLTISNMEAEDAATYYCHQYHLSPWTFGGGTKLEIK

SEQ ID NO: 97 - protein, 107 aa, synthetic construct
DIQMTQTTSSLSASLGDRVTISCRASQDIRNYLNWYQQKPDGTLKLLIYYTSRLHSGVPSRFSGSGSGTDYSLTISNLEQEDVATYFCQQGDPLPPTFGGGTKVEIK

SEQ ID NO: 98 - protein, 107 aa, synthetic construct
DIVMTQSQKFMSTSVGDRVSITCKASQNVDTDVSWYQQKPGKSPKTLIYWASNRFTGVPDRFTGSGSGTDFTLTITNVQSEDLADYFCEQYSSYPYTFGSGTKLEIK

SEQ ID NO: 99 - protein, 106 aa, synthetic construct
ENVLTQSPTIMSASLGEKVTMSCRASSSVSYMHWYQQKSDASPKLWIYYTSNLAPGVPARFSASGSGNSYSLTISSMEGEDAATYYCQQFTSSPYTFGGGTKLEIK

SEQ ID NO: 100 - protein, 107 aa, synthetic construct
DIQMNQSPSSLSASLGDTISISCRASQNINLWLNWYQQKPGNVPKLLIFKASNLHPGVPSRFSGSGSGTDFTLTISSLQPEDIATYYCLQGQSYPWTFGGGTKLEIK

SEQ ID NO: 101 - protein, 107 aa, synthetic construct
DIQMTQTTSSLSASLGDRVTINCRASQDISNFLNWYQQKPDGTLKLLIYYTSGLHSGVPSRFSGSGSGTDYSLTISNLEEEDIATYFCQQDNNHPYTFGSGTKLEIK

SEQ ID NO: 102 - protein, 106 aa, synthetic construct
ENVLTQSPAIMSASLGEKVTMSCRASSSVNYMYWYQQKSDASPKLWIYYTSNLAPGVPARFSGSGSGNSYSLTISSMEGEDAATYYCQQFTSSPWTFGGGTKLEIK

SEQ ID NO: 103 - protein, 107 aa, synthetic construct
DIQMTQSPASLSASVGETVTITCRASEDIDSYLAWYQQKQGKSPQLLVYGATLLADGVPSRFSGSGSGTQYSLKINSLQSEDVARYYCQHYYSIPYTFGSGTKLEIK

SEQ ID NO: 104 - protein, 108 aa, synthetic construct
QIVLTQSPAIMSASPGEKVTMTCSASSSVSSRYLHWYQQKSGASPKLWIYGTSNLASGVPARFSGSGSGTSYSLTISSVEAEDAATYYCQQYHGDPLTFGAGTKLELK

SEQ ID NO: 105 - protein, 108 aa, synthetic construct
QIVLTQSPAIMSASPGEKVTMTCSASSSVSSRYLHWYQQKSGASPKLWIYGTSNLASGVPARFSGSGSGTSYSLTISSVEAEDAASYYCQQYHGDPLTFGAGTKLELK

SEQ ID NO: 106 - protein, 107 aa, synthetic construct
DILLTQSPAILSVSPGERVSLSCRASQTIGTSIHWYQQRTNGSIRLLIKYASESISGIPSRFSGSGFGTDFALSINSVESEDIADYFCQQTNSWPLTFGAGTKLELK

SEQ ID NO: 107 - protein, 106 aa, synthetic construct
QIVLTQSPAIMSASPGEKVTITCSASSRVSYMHWFQQKPGTSPKLWIYSTSNLASGVPARFSGSGSGTSYSLTISRMEAEDAATYYCQQRSSYPLTFGAGTKLELK

SEQ ID NO: 108 - DNA, 363 bp, synthetic construct
  source 1..363: mol_type = other DNA
caggtgcagctgaaggagtcaggacctggcctggtggcgccctcacagagcctgtccatcacttgcaccgtctctgggttttcattaaccagctatggtatagactgggttcgccagcctccaggaaagggtctggagtggctgggaataatgtggggtggtggaagcacaaattataattcagctctcatgtccagactgagcatcagcaaagacatctccaagagccaagttttcttaaaagtgaacagtctgcaaactgatgacacagccatgtactactgtgccagacggacctactctgattcctattactatgaaatggactattggggtcaaggaacctcagtcaccgtctcctca

SEQ ID NO: 109 - DNA, 348 bp, synthetic construct
  source 1..348: mol_type = other DNA
caggtccaactgcagcagcctgggactgagcttgtgaagcctggggcttcagtgaagatgtcctgcaaggcttctggctacaccttcaccagctactggataacctgggtgaagcagaggcctggacaaggccttgagtggattggagatatttatcctggtcgtggtagtactaactacaatgagaagttcaagagcaaggccacactgactgtagacacatcctccagcacagcctacatgcagctcagcagcctgacatctgaggactctgcggtctattactgtgcaagagaactgggagggtttgcttactggggccaagggactctggtcactgtctctgca

SEQ ID NO: 110 - DNA, 363 bp, synthetic construct
  source 1..363: mol_type = other DNA
caggttcagctgcagcagtctggagctgagctgatgaagcctggggcctcagtgaagatttcctgcaaggctactggctccacattcaataggtactggatagagtgggtaaagcagaggcctggacatggccttgagtggattggagagattttacctggaagtggtaatactaattacaatgagaagttcaagggcaaggccacattcactgcagatacatcctccaacacagcctacatgcaactcagcagcctgacatctgaggactctgccgtctattactgtgcaagattggattactacggtagtaggtactactttgactactggggccaaggcaccactctcacagtctcctca

SEQ ID NO: 111 - DNA, 360 bp, synthetic construct
  source 1..360: mol_type = other DNA
gaggtccagctgcaacagtctggacctgaactggtgaagcctggggcttcagtgaagatgtcctgcaaggcttctggattcacattcactgactacaacatacactgggtgaagcagagccatggaaagagccttgagtggattggatatattaaccctaacaatggtcgttctagttacaacctgtatttcaaggacaaggccacattgactgtaaacaagtcgtccagcacagcctacatggagctccgcagcctgacatcggaagattctgcagtctattactgtgcaagagaagactgggagggtttttatgctatggactactggggtcaaggaacctcagtcaccgtctcctca

SEQ ID NO: 112 - DNA, 348 bp, synthetic construct
  source 1..348: mol_type = other DNA
gaggtccagctgcaacagtctggacctgagctggtgaaacctggagcttcaatgaagatatcctgcaaggcttctggttactcattcactgcctacaccatgaactgggtgaggcagagccatggaaagaaccttgagtggattggacttattaatccttacaatggttatgctaactacaaccagaagttcaagggcaaggccacattaactgtagacaagtcatccagtacagcctacatggacctcctcagtctgacatctgaggactctgcagtctattactgtgcaagagtgggatactactttgactactggggccaaggcaccactctcacagtctcctca

SEQ ID NO: 113 - DNA, 339 bp, synthetic construct
  source 1..339: mol_type = other DNA
gaggttcagctgcagcagtctggggctgagcttgtgaggccaggggcctcagtcaagttgtcctgcacatcttctggctttaacattaaagacgactatatgcactgggtgaagcagaggcctgaacagggcctggagtggattggatggattgatcctgagaatggtgatactgaatatgcctcgaagttccagggcaaggccactataacagcagacacatcctccaacacagcctacctgcagctcaacagcctgacatctgaggacactgccgtctattactgtacaggctactttgactactggggccaaggcaccactctcacagtctcctca

SEQ ID NO: 114 - DNA, 348 bp, synthetic construct
  source 1..348: mol_type = other DNA
caggtccaactgcagcagcctggggctgagcttgtgaagcctggggcttcagtgaagatgtcctgcaaggcttctggctacaccttcaccagccactggataacctgggtgaagcagaggcctggacaaggccttgagtggattggagatatttatcctggtagtggtaatactaactacaatgagaagttcaagagcaaggccacactgactgtagacacatcctccagcacagcctacatgcaactcagcagcctgacatctgaggactctgcggactattattgtgcaagagagaggggaggctttgactactggggccaaggcaccactctcacagtctcctca

SEQ ID NO: 115 - DNA, 357 bp, synthetic construct
  source 1..357: mol_type = other DNA
gacgtaatactggtggagtctgggggagacttagtgaagcctggagggtccctgaaactctcctgtgcagcctctggattcactttcagtacctttgccatgtcttgggttcgccagactccggagaagaggctggagtgggtcgcaagcattactggtgatggtggtacttatacctactattcagacagtgtgaagggtcgattcaccatctccagagacaatgccaggaacaccctgtacctgcaaatgagcagtctgaggtctgaggacacggccttctattactgtgcaagacactccgttagtagctggtttgcttactggggccaagggactctggtcactgtctctgca

SEQ ID NO: 116 - DNA, 354 bp, synthetic construct
  source 1..354: mol_type = other DNA
caggttcagctgctacagtctgacgctgagttggtgaaacctggagcttcagtgaagatctcctgcaaggtttctggcttcaccttcactgaccatactcttcactggatgaagcagaggcctgaacagggcctggaatggattggatatatttatcctagagatggttatactaagtacaatgagaaattcaagggcaaggccacattgactgcagacaaatcctccagcacagcctacatgcagctcgacagcctgacatctgaggactctgcagtctatttctgtgcaagaccgacatctttactacggtttccttactggggccaagggactctggtcactgtctctgca

SEQ ID NO: 117 - DNA, 363 bp, synthetic construct
  source 1..363: mol_type = other DNA
caggtgcaactgaaggagtcaggacctggcctggtggcgccctcacagagcctgtccatcacttgcactgtctctgggttttcattaaccagttatggtgtagactgggttcgccagcctccgggaaagggtctggagtggctgggagtaatatggggtggtggtggaagcacaaattataattcagctctcatgtccagactgagcatcagcaaagacaactccaagagtcaagttttcttaaaaatgaacagtctgcaaactgatgacacagccatgtactactgtgccagacataactatgatggttattactattctttggactactggggtcaaggaacctcagtcaccgtctcctca

SEQ ID NO: 118 - DNA, 363 bp, synthetic construct
  source 1..363: mol_type = other DNA
caggtgcagttgaaggagtcaggacctggcctggtggcgccctcacagagcctgtccatcacttgcactgtctctgggttttcattaaccagttatggtgtagactgggttcgccagcctccgggaaagggtctggagtggctgggagtaatatggggtggtggtggaagcacaaattataattcagctctcatgtccagactgagcatcagcaaagacaactccaagagtcaagttttcttaaaaatgaacagtctgcaaactgatgacacagccatgtactactgtgccagacataactatgataattattactatactttggactactggggtcaaggaacctcagtcaccgtctcctca

SEQ ID NO: 119 - DNA, 351 bp, synthetic construct
  source 1..351: mol_type = other DNA
caggtgcaactgaaggagtcaggacctggcctggtggcgccctcacagagcctgtccattacctgcactgtctctgggttctcattaaccagctatgatataagctggattcgccagccaccaggaaagggtctggagtggcttggagtaatatggactggtggaggcacaaattataattcacctttcatgtccagactgaccatcagcaaggacaactccaggagccaagtattcttaaaaatgaacagtctgcaaactgatgacacagccatgtattactgtgtaagagatctcttcccctatgctatggactactggggtcaaggaacctcagtcaccgtctcctca

SEQ ID NO: 120 - DNA, 351 bp, synthetic construct
  source 1..351: mol_type = other DNA
caggtccagcttcagcagtctggggctgaactggcaaaacctggggcctcagtgaagatgtcctgcaaggcttctggctacacctttactgcctactggatacattgggtgaaacagaggcctggacagggtctagaatggattggatacattgatcctaacagtggttatactgactacaatcagatattcaaggacaaggccacattgactgcagacagatcctccagcacagcctacatgcagctgaacagcctgacatctgaggactctgcagtctattactgtgcaagaggacactttggttacgacgactcctggggccaaggcatcactctcacagtctcctca

SEQ ID NO: 121 - DNA, 324 bp, synthetic construct
  source 1..324: mol_type = other DNA
caaattgttctcacccagtctccagcaatcatgtctgcctctccaggggagaaggtcaccatgacctgcagtgccagctcaagtgtaagttccaggtacttgcactggtaccagcagaagtcaggagcctcccccaaactctggatttatggcacatccaacctggcttctggagtccctgctcgcttcagtggcagtgggtctgggacctcttactctctcacaatcagcagcgtggaggctgaagatgctgccacttattactgccagcaatatcatagtgacccgctcacgttcggtgctgggaccaagctggagctgaag

SEQ ID NO: 122 - DNA, 324 bp, synthetic construct
  source 1..324: mol_type = other DNA
caaattgttctcacccagtctccagcaatcatgtctgcatctctaggggaacgggtcaccatgacctgcactgccagctcatatgtaagttccagttacttacactggtaccagcagaagccaggatcctcccccacactctggatttataccacatccaacctggcttctggagtcccagctcgcttcagtggcagtgggtctgggacctcttactctctcacaatcagcaacatggaggctgaagatgctgccacttattactgccaccagtatcatctttccccatggacgttcggtggaggcaccaagctggaaatcaaa

SEQ ID NO: 123 - DNA, 321 bp, synthetic construct
  source 1..321: mol_type = other DNA
gatatccagatgacacagactacatcctccctgtctgcctctctgggagacagagtcaccatcagttgcagggcaagtcaggacattaggaattatttaaactggtatcagcagaaaccagatggaactcttaaactcctgatctattacacatcaagattacactcaggagtcccatcaaggttcagtggcagtgggtctggaacagattattctctcaccattagcaacctggagcaagaagatgttgccacttacttttgccaacagggtgatccgcttcctccgacgttcggtggaggcaccaaggtggaaatcaaa

SEQ ID NO: 124 - DNA, 321 bp, synthetic construct
  source 1..321: mol_type = other DNA
gacattgtgatgacccagtctcaaaaattcatgtccacatcagtaggagacagggtcagcatcacctgcaaggccagtcagaatgtggatactgatgtatcctggtatcaacagaaaccagggaaatctcctaaaacactgatttattgggcatcaaaccggttcactggagtccctgatcgcttcacaggcagtggatctgggacagatttcactctcaccatcaccaatgtgcagtctgaagacttggcagattatttctgtgagcaatatagcagctatccgtatacgttcggatcggggaccaagctggaaataaaa

SEQ ID NO: 125 - DNA, 318 bp, synthetic construct
  source 1..318: mol_type = other DNA
gaaaatgtgctcacccagtctccaacaatcatgtctgcatctctaggggagaaggtcaccatgagctgcagggccagctcaagtgttagttacatgcactggtaccagcagaagtcagatgcctcccccaaactttggatttattacacatccaacctggctcctggagtcccagctcgcttcagtgccagtgggtctgggaactcttattctctcacaatcagcagcatggagggtgaagatgctgccacttattactgccagcagtttactagttccccatacacgttcggaggggggaccaagctggaaataaaa

SEQ ID NO: 126 - DNA, 321 bp, synthetic construct
  source 1..321: mol_type = other DNA
gacatccagatgaaccagtctccatccagtctgtctgcatccctcggagacacaatttccatctcttgccgtgccagtcagaacattaatctttggttaaactggtaccagcagaaaccaggaaatgttcctaaactattgatctttaaggcttccaacttgcacccaggcgtcccatcaaggtttagtggcagtggatctggaacagatttcacattaaccatcagcagtctgcagcctgaagacattgccacttactactgtctacagggtcaaagttatccgtggacgttcggtggaggcaccaagctggaaatcaaa

SEQ ID NO: 127 - DNA, 321 bp, synthetic construct
  source 1..321: mol_type = other DNA
gatatccagatgacacagactacatcttccctgtctgcctctctgggagacagagtcaccatcaattgcagggcaagtcaggacattagcaattttttaaactggtatcagcagaaaccagatggaactcttaaactcctgatctactacacatcaggattacactcaggagtcccatcaaggttcagtggcagtgggtctgggacagattattctctcactattagcaacctggaggaagaagatattgccacttacttttgccaacaggataataaccatccttatacgttcggatcggggaccaagctggaaataaaa

SEQ ID NO: 128 - DNA, 318 bp, synthetic construct
  source 1..318: mol_type = other DNA
gaaaatgtgctcacccagtctccagcaatcatgtctgcatctctaggggagaaggtcaccatgagctgcagggccagctcaagtgtaaattacatgtactggtaccagcagaagtcagatgcctcccccaaactatggatttattacacatccaacctggctcctggagtcccagctcgcttcagtggcagtgggtctgggaactcttattctctcacaatcagcagcatggagggtgaagatgctgccacttattactgccagcagtttactagttccccgtggacgttcggtggaggcaccaagctggaaatcaaa

SEQ ID NO: 129 - DNA, 321 bp, synthetic construct
  source 1..321: mol_type = other DNA
gacatccagatgactcagtctccagcttccctgtctgcatctgtgggagaaactgtcaccatcacatgtcgagcaagtgaagatattgacagttatttagcttggtatcaacagaaacagggaaaatctcctcagctcctggtctatggtgcaacactcttagcagatggtgtgccatcaaggttcagtggcagtggatcaggcacacagtattctctcaagatcaacagcctgcagtctgaagatgttgcgagatattactgtcaacattattatagtattccgtatacgttcggatcggggaccaagctggaaataaaa

SEQ ID NO: 130 - DNA, 324 bp, synthetic construct
  source 1..324: mol_type = other DNA
caaattgttctcacccagtctccagcaatcatgtctgcctctccaggggagaaggtcaccatgacctgcagtgccagctcaagtgtaagttccaggtacttgcactggtaccagcagaagtcaggagcctcccccaaactctggatttatggcacatccaacctggcttctggagtccctgctcgcttcagtggcagtgggtctgggacctcttactctctcacaatcagcagcgtggaggctgaagatgctgccacttattactgccagcagtatcatggtgacccgctcacgttcggtgctgggaccaagctggagctgaaa

SEQ ID NO: 131 - DNA, 324 bp, synthetic construct
  source 1..324: mol_type = other DNA
caaattgttctcacccagtctccagcaatcatgtctgcctctccaggggagaaggtcaccatgacctgcagtgccagctcaagtgtaagttccaggtacttgcactggtaccagcagaagtcaggagcctcccccaaactctggatttatggcacatccaacctggcttctggagtccctgctcgcttcagtggcagtgggtctgggacctcttactctctcacaatcagcagcgtggaggctgaagatgctgcctcttattactgccagcagtatcatggtgacccgctcacgttcggtgctgggaccaagctggagctgaaa

SEQ ID NO: 132 - DNA, 321 bp, synthetic construct
  source 1..321: mol_type = other DNA
gacatcctgctgactcagtctccagccatcctgtctgtgagtccaggagaaagagtcagcctctcctgcagggccagtcagaccattggcacaagcatacactggtatcagcaaagaacaaatggttctataaggcttctcataaaatatgcttctgagtctatctctgggatcccttctaggtttagtggcagtggatttgggacagattttgctcttagcatcaacagtgtggagtctgaagatattgcagattatttctgtcaacaaactaatagctggccactcacgttcggtgctgggaccaagctggagctgaaa

SEQ ID NO: 133 - DNA, 318 bp, synthetic construct
  source 1..318: mol_type = other DNA
caaattgttctcacccagtctccagcaatcatgtctgcatctccaggggagaaggtcaccataacctgcagtgccagctcaagagtaagttacatgcactggttccaacaaaagccaggcacttctcccaaactctggatttatagcacatccaacctggcttctggagtccctgctcgcttcagtggcagtggatctgggacctcttattctttaacaatcagccgaatggaggctgaagatgctgccacttattactgccagcaaaggagtagttacccactcacgttcggtgctgggaccaagctggagctgaaa

SEQ ID NO: 134 - protein, 485 aa, synthetic construct
  VARIANT 1..40: note = Any amino acid
  VARIANT 15..40: note = Residues may be deleted
XXXXXXXXXXXXXXXXXXXXXXXXXXXXXXXXXXXXXXXXQVQLQQPGAELVKPGASVKMSCKASGYTFTSHWITWVKQRPGQGLEWIGDIYPGSGNTNYNEKFKSKATLTVDTSSSTAYMQLSSLTSEDSADYYCARERGGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 485 aa, synthetic construct
  VARIANT 1..40: note = Any amino acid
  VARIANT 15..40: note = Residues may be deleted
XXXXXXXXXXXXXXXXXXXXXXXXXXXXXXXXXXXXXXXXQVQLVQSGAEVKKPGASVKVSCKASGYTFTSHWISWVRQAPGQGLEWMGDIYPGSGNTNYNEKLQGRVTMTVDTSTSTAYMELRSLRSDDTAVYYCARERGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 136 - protein, 485 aa, synthetic construct
  VARIANT 1..40: note = Any amino acid
  VARIANT 15..40: note = Residues may be deleted
XXXXXXXXXXXXXXXXXXXXXXXXXXXXXXXXXXXXXXXXQVQLVQSGAEVKKPGASVKVSCKASGYTFTSHWITWVRQAPGQGLEWMGDIYPGSGNTNYNEKFQGRVTLTVDTSISTAYMELSRLRSDDTVVYYCARERGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 485 aa, synthetic construct
  VARIANT 1..40: note = Any amino acid
  VARIANT 15..40: note = Residues may be deleted
XXXXXXXXXXXXXXXXXXXXXXXXXXXXXXXXXXXXXXXXQVQLVQSGAEVKKPGASVKVSCKASGYTFTSHWITWVRQAPGQRLEWMGDIYPGSGNTNYNEKFQGRVTITVDTSASTAYMELSSLRSEDTAVYYCARERGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 138 - protein, 482 aa, synthetic construct
  VARIANT 1..40: note = Any amino acid
  VARIANT 15..40: note = Residues may be deleted
XXXXXXXXXXXXXXXXXXXXXXXXXXXXXXXXXXXXXXXXQVQLQQPGAELVKPGASVKMSCKASGYTFTSHWITWVKQRPGQGLEWIGDIYPGSGNTNYNEKFKSKATLTVDTSSSTAYMQLSSLTSEDSADYYCARERGGFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 139 - protein, 482 aa, synthetic construct
  VARIANT 1..40: note = Any amino acid
  VARIANT 15..40: note = Residues may be deleted
XXXXXXXXXXXXXXXXXXXXXXXXXXXXXXXXXXXXXXXXQVQLVQSGAEVKKPGASVKVSCKASGYTFTSHWISWVRQAPGQGLEWMGDIYPGSGNTNYNEKLQGRVTMTVDTSTSTAYMELRSLRSDDTAVYYCARERGGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 140 - protein, 482 aa, synthetic construct
  VARIANT 1..40: note = Any amino acid
  VARIANT 15..40: note = Residues may be deleted
XXXXXXXXXXXXXXXXXXXXXXXXXXXXXXXXXXXXXXXXQVQLVQSGAEVKKPGASVKVSCKASGYTFTSHWITWVRQAPGQGLEWMGDIYPGSGNTNYNEKFQGRVTLTVDTSISTAYMELSRLRSDDTVVYYCARERGGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 141 - protein, 482 aa, synthetic construct
  VARIANT 1..40: note = Any amino acid
  VARIANT 15..40: note = Residues may be deleted
XXXXXXXXXXXXXXXXXXXXXXXXXXXXXXXXXXXXXXXXQVQLVQSGAEVKKPGASVKVSCKASGYTFTSHWITWVRQAPGQRLEWMGDIYPGSGNTNYNEKFQGRVTITVDTSASTAYMELSSLRSEDTAVYYCARERGGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 142 - protein, 254 aa, synthetic construct
  VARIANT 1..40: note = Any amino acid
  VARIANT 15..40: note = Residues may be deleted
XXXXXXXXXXXXXXXXXXXXXXXXXXXXXXXXXXXXXXXXDIQMTQTTSSLSASLGDRVTINCRASQDISNFLNWYQQKPDGTLKLLIYYTSGLHSGVPSRFSGSGSGTDYSLTISNLEEEDIATYFCQQDNNHPYTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 254 aa, synthetic construct
  VARIANT 1..40: note = Any amino acid
  VARIANT 15..40: note = Residues may be deleted
XXXXXXXXXXXXXXXXXXXXXXXXXXXXXXXXXXXXXXXXDIQMTQSPSSLSASVGDRVTITCRASQDISNFLNWYQQKPEGTLKLLIYYTSGLHSGVPSRFSGSGSGTDFTLTISSLQPEDVATYYCQQDNNHPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 254 aa, synthetic construct
  VARIANT 1..40: note = Any amino acid
  VARIANT 15..40: note = Residues may be deleted
XXXXXXXXXXXXXXXXXXXXXXXXXXXXXXXXXXXXXXXXDIQMTQSPSSLSASVGDRVTITCQASQDISNFLNWYQQKPGKTLKLLIYYTSGLHTGVPSRFSGSGSGTDFTFTISSLQPEDIATYYCQQDNNHPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 254 aa, synthetic construct
  VARIANT 1..40: note = Any amino acid
  VARIANT 15..40: note = Residues may be deleted
XXXXXXXXXXXXXXXXXXXXXXXXXXXXXXXXXXXXXXXXDIQMTQSPSSLSASVGDRVTITCRASQDISNFLNWYQQKPGKAPKLLIYYTSGLHSGVPSRFSGSGSGTDFTLTISSLQPEDFATYYCQQDNNHPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - DNA, 1458 bp, synthetic construct
  source 1..1458: mol_type = other DNA
  misc_difference 43..120: note = Residues may be deleted
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaggtgcagctccagcagcccggagcggagctggttaagcctggcgcaagcgtgaagatgagttgcaaggccagcggctacaccttcaccagccactggatcacctgggtgaagcagaggccgggacagggcctggagtggatcggcgacatctaccccggcagcggcaacaccaactacaacgagaagttcaagagtaaggccacactgaccgtggacaccagtagcagcaccgcctacatgcagctgagcagcctgaccagcgaggacagcgccgactactactgtgcccgcgagaggggcggattcgactattggggccaaggcaccaccttgaccgtcagctctgctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgaagagatgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatag

SEQ ID NO: 147 - DNA, 1458 bp, synthetic construct
  source 1..1458: mol_type = other DNA
  misc_difference 43..120: note = Residues may be deleted
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaggtgcagctggtgcagagcggagcagaggtgaagaagcccggagcgagcgtgaaggtgagctgcaaggccagcggctacacctttaccagccactggatcagctgggtgaggcaagcccctggacagggcctggagtggatgggcgacatctaccccggcagcggcaacacaaactacaacgagaagctgcagggcagggtgaccatgaccgtggacaccagtaccagcacagcctacatggagctgaggagcctcaggagcgacgacaccgccgtgtactactgcgccagggaaaggggcggattcgactactggggccagggcaccctggtgaccgttagcagcgctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgaagagatgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatag

SEQ ID NO: 148 - DNA, 1458 bp, synthetic construct
  source 1..1458: mol_type = other DNA
  misc_difference 43..120: note = Residues may be deleted
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaggtgcagctggtgcagagcggagcagaggtgaagaagcccggagcgagcgtgaaggtgagctgcaaggccagcggctacacctttaccagccactggatcacctgggtgaggcaagcccctggacagggcctggagtggatgggcgacatctaccccggcagcggcaacacaaactacaacgagaagttccagggcagggtgaccttgaccgtggacaccagtatcagcacagcctacatggagctgagtagactcaggagcgacgacaccgtcgtgtactactgcgccagggaaaggggcggattcgactactggggccagggcaccctggtgaccgttagcagcgctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgaagagatgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatag

SEQ ID NO: 149 - DNA, 1458 bp, synthetic construct
  source 1..1458: mol_type = other DNA
  misc_difference 43..120: note = Residues may be deleted
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaggtgcagctggtgcagagcggagcagaggtgaagaagcccggagcgagcgtgaaggtgagctgcaaggccagcggctacacctttaccagccactggatcacctgggtgaggcaagcccctggacagcgactggagtggatgggcgacatctaccccggcagcggcaacacaaactacaacgagaagttccagggcagggtgaccatcaccgtggacaccagtgccagcacagcctacatggagctgagtagcctcaggagcgaagacaccgccgtgtactactgcgccagggaaaggggcggattcgactactggggccagggcaccctggtgaccgttagcagcgctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgaagagatgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatag

SEQ ID NO: 150 - DNA, 1449 bp, synthetic construct
  source 1..1449: mol_type = other DNA
  misc_difference 43..120: note = Residues may be deleted
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaggtgcagctccagcagcccggagcggagctggttaagcctggcgcaagcgtgaagatgagttgcaaggccagcggctacaccttcaccagccactggatcacctgggtgaagcagaggccgggacagggcctggagtggatcggcgacatctaccccggcagcggcaacaccaactacaacgagaagttcaagagtaaggccacactgaccgtggacaccagtagcagcaccgcctacatgcagctgagcagcctgaccagcgaggacagcgccgactactactgtgcccgcgagaggggcggattcgactattggggccaaggcaccaccttgaccgtcagctctgctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctag

SEQ ID NO: 151 - DNA, 1449 bp, synthetic construct
  source 1..1449: mol_type = other DNA
  misc_difference 43..120: note = Residues may be deleted
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaggtgcagctggtgcagagcggagcagaggtgaagaagcccggagcgagcgtgaaggtgagctgcaaggccagcggctacacctttaccagccactggatcagctgggtgaggcaagcccctggacagggcctggagtggatgggcgacatctaccccggcagcggcaacacaaactacaacgagaagctgcagggcagggtgaccatgaccgtggacaccagtaccagcacagcctacatggagctgaggagcctcaggagcgacgacaccgccgtgtactactgcgccagggaaaggggcggattcgactactggggccagggcaccctggtgaccgttagcagcgctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctag

SEQ ID NO: 152 - DNA, 1449 bp, synthetic construct; identical to SEQ ID NO: 151
  source 1..1449: mol_type = other DNA
  misc_difference 43..120: note = Residues may be deleted

SEQ ID NO: 153 - DNA, 1449 bp, synthetic construct
  source 1..1449: mol_type = other DNA
  misc_difference 43..120: note = Residues may be deleted
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaggtgcagctggtgcagagcggagcagaggtgaagaagcccggagcgagcgtgaaggtgagctgcaaggccagcggctacacctttaccagccactggatcacctgggtgaggcaagcccctggacagcgactggagtggatgggcgacatctaccccggcagcggcaacacaaactacaacgagaagttccagggcagggtgaccatcaccgtggacaccagtgccagcacagcctacatggagctgagtagcctcaggagcgaagacaccgccgtgtactactgcgccagggaaaggggcggattcgactactggggccagggcaccctggtgaccgttagcagcgctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctag

SEQ ID NO: 154 - DNA, 765 bp, synthetic construct
  source 1..765: mol_type = other DNA
  misc_difference 43..120: note = Residues may be deleted
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngacatccagatgacccagaccaccagcagcctgagcgccagcctgggcgacagggtgaccatcaactgcagggccagccaggacataagcaacttcctgaactggtatcaacagaagcccgacggcaccctgaagctgctgatctactacaccagcggcctgcacagcggcgtgcccagtaggttcagcggtagcggctccggcaccgattatagcctgactatcagcaacctggaggaagaggatatcgccacctacttctgccagcaggacaacaaccacccctacacctttggcagcggcaccaagttggagatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 155 - DNA, 765 bp, synthetic construct
  source 1..765: mol_type = other DNA
  misc_difference 43..120: note = Residues may be deleted
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngacatccagatgacccagagccccagctccctgagcgccagcgtgggcgacagggtgaccatcacgtgcagggccagccaggacatcagcaacttcctgaactggtatcaacagaagcccgaaggcaccctgaagctgcttatatactacaccagcggcctgcacagcggcgtgcccagccgattcagcggcagcgggagtggcaccgacttcacccttacaatcagcagtctgcagcccgaggacgtggctacctactactgccagcaggataacaaccacccctacaccttcggccagggcactaagctggagatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 156 - DNA, 765 bp, synthetic construct
  source 1..765: mol_type = other DNA
  misc_difference 43..120: note = Residues may be deleted
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngacatccagatgacccagagccccagctccctgagcgccagcgtgggcgacagggtgaccatcacgtgccaggccagccaggacatcagcaacttcctgaactggtatcaacagaagcccggaaagaccctgaagctgcttatatactacaccagcggcctgcacaccggcgtgcccagccgattcagcggcagcgggagtggcaccgacttcacctttacaatcagcagtctgcagcccgaggacattgctacctactactgccagcaggataacaaccacccctacaccttcggccagggcactaagctggagatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 157 - DNA, 765 bp, synthetic construct
  source 1..765: mol_type = other DNA
  misc_difference 43..120: note = Residues may be deleted
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngacatccagatgacccagagccccagctccctgagcgccagcgtgggcgacagggtgaccatcacgtgcagggccagccaggacatcagcaacttcctgaactggtatcaacagaagcccggaaaggctccgaagctgcttatatactacaccagcggcctgcacagcggcgtgcccagccgattcagcggcagcgggagtggcaccgacttcacccttacaatcagcagtctgcagcccgaggacttcgctacctactactgccagcaggataacaaccacccctacaccttcggccagggcactaagctggagatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 158 - protein, 24 aa, synthetic construct
MDPKGSLSWRILLFLSLAFELSYG

SEQ ID NO: 159 - protein, 20 aa, synthetic construct
METDTLLLWVLLLWVPGSTG

SEQ ID NO: 160 - protein, 19 aa, synthetic construct
MGWSLILLFLVAVATRVHS

SEQ ID NO: 161 - protein, 20 aa, synthetic construct
MRVPAQLLGLLLLWLPGARC

SEQ ID NO: 162 - protein, 5 aa, synthetic construct
SHWIT

SEQ ID NO: 163 - protein, 10 aa, synthetic construct
GYTFTSHWIT

SEQ ID NO: 164 - protein, 7 aa, synthetic construct
GYTFTSH

SEQ ID NO: 165 - protein, 6 aa, synthetic construct
TSHWIT

SEQ ID NO: 166 - protein, 5 aa, synthetic construct
SHWIS

SEQ ID NO: 167 - protein, 10 aa, synthetic construct
GYTFTSHWIS

SEQ ID NO: 168 - protein, 6 aa, synthetic construct
TSHWIS

SEQ ID NO: 169 - protein, 17 aa, synthetic construct
DIYPGSGNTNYNEKFKS

SEQ ID NO: 170 - protein, 10 aa, synthetic construct
DIYPGSGNTN

SEQ ID NO: 171 - protein, 6 aa, synthetic construct
YPGSGN

SEQ ID NO: 172 - protein, 13 aa, synthetic construct
WIGDIYPGSGNTN

SEQ ID NO: 173 - protein, 17 aa, synthetic construct
DIYPGSGNTNYNEKLQG

SEQ ID NO: 174 - protein, 13 aa, synthetic construct
WMGDIYPGSGNTN

SEQ ID NO: 175 - protein, 17 aa, synthetic construct
DIYPGSGNTNYNEKFQG

SEQ ID NO: 176 - protein, 7 aa, synthetic construct
ERGGFDY

SEQ ID NO: 177 - protein, 8 aa, synthetic construct
ARERGGFD

SEQ ID NO: 178 - protein, 11 aa, synthetic construct
RASQDISNFLN

SEQ ID NO: 179 - protein, 7 aa, synthetic construct
SNFLNWY

SEQ ID NO: 180 - protein, 11 aa, synthetic construct
QASQDISNFLN

SEQ ID NO: 181 - protein, 7 aa, synthetic construct
YTSGLHS

SEQ ID NO: 182 - protein, 10 aa, synthetic construct
LLIYYTSGLH

SEQ ID NO: 183 - protein, 7 aa, synthetic construct
YTSGLHT

SEQ ID NO: 184 - protein, 8 aa, synthetic construct
QQDNNHPY

SEQ ID NO: 185 - protein, 539 aa, Mus sp.
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSHYIEPYLEFEARRRLLGHSWEDASVLSLKQRQQWLFLEMLIPSTSYEVQVRVKAQRNNTGTWSPWSQPLTFRTRPADPMKEILPMSWLRYLLLVLGCFSGFFSCVYILVKCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 186 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 187 - protein, 214 aa, Homo sapiens
AAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 188 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 189 - protein, 5 aa, synthetic construct
DFYME

SEQ ID NO: 190 - protein, 19 aa, synthetic construct
ASRNKANDYTTEYSASVKG

SEQ ID NO: 191 - protein, 10 aa, synthetic construct
SYYRYDGMDY

SEQ ID NO: 192 - protein, 10 aa, synthetic construct
SAISSVSYMY

SEQ ID NO: 193 - protein, 7 aa, synthetic construct
DTSNLVS

SEQ ID NO: 194 - protein, 9 aa, synthetic construct
QQWNTYPYT

SEQ ID NO: 195 - protein, 6 aa, synthetic construct
VDIVTL

SEQ ID NO: 196 - protein, 7 aa, synthetic construct
FKPFENL

SEQ ID NO: 197 - protein, 6 aa, synthetic construct
HYFERH

SEQ ID NO: 198 - protein, 5 aa, synthetic construct
DGTLK

(19) United States
(12) Patent Application Publication
(10) Pub. No.: US 2025/0257143 A1
(43) Pub. Date: Aug. 14, 2025

(54) ANTI-CD122 ANTIBODIES AND USES THEREOF

(71) Applicant: FORTE SUBSIDIARY, INC., Dallas, TX (US)

(72) Inventor: Paul A. WAGNER, San Juan, PR (US)

(21) Appl. No.: 19/094,089

(22) Filed: Mar. 28, 2025

Related U.S. Application Data
(63) Continuation of application No. PCT/US2023/075600, filed on Sep. 29, 2023.
(60) Provisional application No. 63/377,847, filed on Sep. 30, 2022.

(51) Int. Cl. C07K 16/28

(52) U.S. Cl. CPC C07K 16/2866; C07K 2317/24; C07K 2317/76; C07K 2317/92

(57) ABSTRACT

Provided herein, in certain aspects, are anti-CD122 antibodies and pharmaceutical compositions which comprise anti-CD122 antibodies. In some embodiments, the anti-CD122 antibodies and the pharmaceutical compositions comprising anti-CD122 antibodies can be used for targeting CD122-expressing tissues and cells.

### CROSS-REFERENCE TO RELATED APPLICATIONS

This application is a continuation of International Patent Application No. PCT/US2023/075600 filed Sep. 29, 2023, which claims the benefit of U.S. provisional application Ser. No. 63/377,847 filed Sep. 30, 2022; which are hereby incorporated by reference in their entireties.

### INCORPORATION BY REFERENCE OF SEQUENCE LISTING

The present application is being filed along with a Sequence Listing in electronic format. The Sequence Listing is provided as a file entitled 53654-717.301_SL.XML, created Mar. 28, 2025, which is 247,841 bytes in size. The information in the electronic format of the Sequence Listing is incorporated by reference in its entirety.

### BACKGROUND

Interleukin receptors are involved in mediating many cellular responses including T cell immune responses. The interleukin 2 receptor is present in three forms with respect to ability to bind interleukin 2 (IL2). The low affinity form of the receptor is a monomer of the alpha receptor subunit (IL2RA; also known as CD25) and is not involved in signal transduction. The intermediate affinity receptor form is composed of an alpha/beta subunit heterodimer, while the high affinity receptor form is composed of an alpha/beta/gamma subunit heterotrimer. Both the intermediate and high affinity forms of the receptor are involved in receptor-mediated endocytosis and transduction pathways for IL2. The IL2RB gene encodes the beta subunit of the interleukin 2 receptor. The protein encoded by the IL2RB gene (IL2RB) is a type I transmembrane protein with its amino- (N-) terminal domains extracellular to the plasma membrane in mature forms. IL2RB is also known as IL15RB or CD122. CD122 protein is primarily expressed in the hematopoietic system. The gamma subunit of the IL2 receptor is IL2RG (also known as CD132).

In addition to functioning in IL2-mediating signaling as the beta receptor subunit, CD122 also transmits signals from the cytokine interleukin 15 (IL15). Unlike the alpha subunit of the IL2 receptor, the alpha subunit of the IL15 receptor (IL15RA or IL15Ra) is capable of binding its ligand (IL15) with high affinity independent of the other receptor subunits. IL15RA can form an alpha/beta subunit heterodimer together with CD122 for signal transduction. IL15RA can also form an alpha/beta/gamma subunit heterotrimer with CD122 and CD132 for signal transduction. It is through these various interleukin receptor complexes that CD122 is involved in transmitting signals from the cytokines IL2 and IL15.

As such, anti-CD122 antibodies can be used for diagnostics, as well as therapeutic purposes for diseases or symptoms related to abundant CD122 expression. Thus, there is a need for developing improved anti-CD122 antibodies.

### SUMMARY

Disclosed herein, in certain aspects, are anti-CD122 antibodies, and pharmaceutical compositions which comprise the anti-CD122 antibodies. In some embodiments, the anti-CD122 antibodies and the pharmaceutical compositions comprising anti-CD122 antibodies can be used for targeting CD122-expressing tissues and cells.

Described herein, in some embodiments, are anti-CD122 antibodies comprising i) a heavy chain comprising a variable heavy chain (VH) domain and ii) a light chain comprising a variable light chain (VL) domain, wherein the VH domain comprises an HCDR1 sequence comprising a sequence selected from SEQ ID NOs: 1-11, an HCDR2 sequence comprising a sequence selected from SEQ ID NOs: 12-23, and an HCDR3 sequence comprising a sequence selected from SEQ ID NOs: 24-36, and VL domain comprises an LCDR1 sequence comprising a sequence selected from SEQ ID NOs: 37-47, an LCDR2 sequence comprising a sequence selected from GTS, TTS, YTS, WAS, KAS, GAT, YAS or STS, and an LCDR3 sequence comprising a sequence selected from SEQ ID NOs: 56-67. In some embodiments, the anti-CD122 antibody comprises the HCDR1 sequence comprising SEQ ID NO: 1, the HCDR2 sequence comprising SEQ ID NO: 12, the HCDR3 sequence comprising SEQ ID NO: 24, the LCDR1 sequence comprising SEQ ID NO: 37, the LCDR2 sequence comprising GTS, and the LCDR3 sequence comprising SEQ ID NO: 56. In some embodiments, the anti-CD122 antibody comprises the HCDR1 sequence comprising SEQ ID NO: 2, the HCDR2 sequence comprising SEQ ID NO: 13, the HCDR3 sequence comprising SEQ ID NO: 25, the LCDR1 sequence comprising SEQ ID NO: 38, the LCDR2 sequence comprising TTS, and the LCDR3 sequence comprising SEQ ID NO: 57. In some embodiments, the anti-CD122 antibody comprises the HCDR1 sequence comprising SEQ ID NO: 3, the HCDR2 sequence comprising SEQ ID NO: 14, the HCDR3 sequence comprising SEQ ID NO: 26, the LCDR1 sequence comprising SEQ ID NO: 39, the LCDR2 sequence comprising YTS, and the LCDR3 sequence comprising SEQ ID NO: 58. In some embodiments, the anti-CD122 antibody comprises wherein the HCDR1 sequence comprising SEQ ID NO: 4, the HCDR2 sequence comprising SEQ ID NO: 15, the HCDR3 sequence comprising SEQ ID NO: 27, the LCDR1 sequence comprising SEQ ID NO: 40, the LCDR2 sequence comprising WAS, and the LCDR3 comprising SEQ ID NO: 59. In some embodiments, the anti-CD122 antibody comprises the HCDR1 sequence comprising SEQ ID NO: 5, the HCDR2 sequence comprising SEQ ID NO: 16, the HCDR3 sequence comprising SEQ ID NO: 28, the LCDR1 sequence comprising SEQ ID NO: 41, the LCDR2 sequence comprising YTS, and the LCDR3 sequence comprising SEQ ID NO: 60. In some embodiments, the anti-CD122 antibody comprises the HCDR1 sequence comprising SEQ ID NO: 6, the HCDR2 sequence comprising SEQ ID NO: 17, the HCDR3 sequence comprising SEQ ID NO: 29, the LCDR1 sequence comprising SEQ ID NO: 42, the LCDR2 sequence comprising KAS, and the LCDR3 sequence comprising SEQ ID NO: 61. In some embodiments, the anti-CD122 antibody comprises the HCDR1 sequence comprising SEQ ID NO: 7, the HCDR2 sequence comprising SEQ ID NO: 18, the HCDR3 sequence comprising SEQ ID NO: 30, the LCDR1 sequence comprising SEQ ID NO: 43, the LCDR2 sequence comprising YTS, and the LCDR3 sequence comprising SEQ ID NO: 62. In some embodiments, the anti-CD122 antibody comprises the HCDR1 sequence comprising SEQ ID NO: 8, the HCDR2 sequence comprising SEQ ID NO: 19, the HCDR3 sequence comprising SEQ ID NO: 31, the LCDR1 sequence comprising SEQ ID NO: 44, the LCDR2 sequence comprising YTS, and the LCDR3 sequence comprising SEQ ID NO: 63. In some embodiments, the anti-CD122 antibody comprises the HCDR1 sequence comprising SEQ ID NO: 9, the HCDR2 sequence comprising SEQ ID NO: 20, the HCDR3 sequence comprising SEQ ID NO: 32, the LCDR1 sequence comprising SEQ ID NO: 45, the LCDR2 sequence comprising GAT, and the LCDR3 sequence comprising SEQ ID NO: 64. In some embodiments, the anti-CD122 antibody comprises the HCDR1 sequence comprising SEQ ID NO: 1, the HCDR2 sequence comprising SEQ ID NO: 21, the HCDR3 sequence comprising SEQ ID NO: 33, the LCDR1 sequence comprising SEQ ID NO: 37, the LCDR2 sequence comprising GTS, and the LCDR3 sequence comprising SEQ ID NO: 65. In some embodiments, the anti-CD122 antibody comprises the HCDR1 sequence comprising SEQ ID NO: 1, the HCDR2 sequence comprising SEQ ID NO: 21, the HCDR3 sequence comprising SEQ ID NO: 34, the LCDR1 sequence comprising SEQ ID NO: 37, the LCDR2 sequence comprising GTS, and the LCDR3 sequence comprising SEQ ID NO: 65. In some embodiments, the anti-CD122 antibody comprises the HCDR1 sequence comprising SEQ ID NO: 10, the HCDR2 sequence comprising SEQ ID NO: 22, the HCDR3 sequence comprising SEQ ID NO: 35, the LCDR1 sequence comprising SEQ ID NO: 46, the LCDR2 sequence comprising YAS, and the LCDR3 sequence comprising SEQ ID NO: 66. In some embodiments, the anti-CD122 antibody comprises the HCDR1 sequence comprising SEQ ID NO: 11, the HCDR2 sequence comprising SEQ ID NO: 23, the HCDR3 sequence comprising SEQ ID NO: 36, the LCDR1 sequence comprising SEQ ID NO: 47, the LCDR2 sequence comprising STS, and the LCDR3 sequence comprising SEQ ID NO: 67. In some embodiments, the anti-CD122 antibody comprises the VH domain comprising at least 80%, at least 85%, at least 90%, or at least 95% sequence identity to a sequence selected from SEQ ID NOs: 82-94. In some embodiments, the anti-CD122 antibody comprises the VL domain comprising at least 80%, at least 85%, at least 90%, or at least 95% sequence identity to a sequence selected from SEQ ID NOs: 95-107. In some embodiments, the anti-CD122 antibody comprises the VH domain encoded by a nucleic acid comprising at least 80%, at least 85%, at least 90%, or at least 95% sequence identity to a sequence selected from SEQ ID NOs: 108-120. In some embodiments, the anti-CD122 antibody comprises the VL domain encoded by a nucleic acid comprising at least 80%, at least 85%, at least 90%, or at least 95% sequence identity to a sequence selected from SEQ ID NOs: 121-133. In some embodiments, the anti-CD122 antibody comprises the heavy chain comprising at least 80%, at least 85%, at least 90%, or at least 95% sequence identity to a sequence selected from SEQ ID NOs: 134-141. In some embodiments, the anti-CD122 antibody comprises the light chain comprising at least 80%, at least 85%, at least 90%, or at least 95% sequence identity to a sequence selected from SEQ ID NOs: 142-145. In some embodiments, the anti-CD122 antibody comprises the heavy chain encoded by a nucleic acid comprising at least 80%, at least 85%, at least 90%, or at least 95% sequence identity to a sequence selected from SEQ ID NOs: 146-153. In some embodiments, the anti-CD122 antibody comprises the light chain encoded by a nucleic acid comprising at least 80%, at least 85%, at least 90%, or at least 95% sequence identity to a sequence selected from SEQ ID NOs: 154-157. In some embodiments, the anti-CD122 antibody comprises the heavy chain comprising a leader sequence at an N-terminus of a heavy chain polypeptide. In some embodiments, the anti-CD122 antibody comprises the heavy chain wherein the leader sequence comprises SEQ ID NO: 158, SEQ ID NO: 159. SEQ ID NO: 160, or SEQ ID NO: 161. In some embodiments, the anti-CD122 antibody comprises the light chain comprising a leader sequence at an N-terminus of a light chain polypeptide. In some embodiments, the anti-CD122 antibody comprises the light chain wherein the leader sequence comprises SEQ ID NO: 158, SEQ ID NO: 159. SEQ ID NO: 160, or SEQ ID NO: 161. In some embodiments, the anti-CD122 antibody is a humanized antibody or antigen binding fragment thereof. In some embodiments, the anti-CD122 antibody is a chimeric antibody or antigen binding fragment thereof. In some embodiments, the anti-CD122 antibody comprises an IgG-scFv, nanobody, mini-antibody, minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, or intrabody. In some embodiments, the anti-CD122 antibody is an IgG1 antibody. In some embodiments, the anti-CD122 antibody is an IgG2 antibody. In some embodiments, the anti-CD122 antibody is an IgG4 antibody. In some embodiments, the anti-CD122 antibody comprises a light chain wherein the light chain is a kappa chain. In some embodiments, the anti-CD122 antibody has a binding affinity to human CD122 of about 100 pM to about 3 nM. In some embodiments, a pharmaceutical composition described herein comprises an anti-CD122 antibody described herein and a pharmaceutically acceptable excipient.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows sensorgram plots of anti-CD122 antibodies G1 (top) and G2 (bottom) to measure binding and dissociation properties to different concentrations of huCD122.

FIG. 2 shows sensorgram plots of anti-CD122 antibodies G3 (top) and G4 (bottom) to measure binding and dissociation properties to different concentrations of huCD122.

FIG. 3 shows sensorgram plots of anti-CD122 antibodies G5 (top) and G6 (bottom) to measure binding and dissociation properties to different concentrations of huCD122.

FIG. 4 shows sensorgram plots of anti-CD122 antibodies G7 (top) and G8 (bottom) to measure binding and dissociation properties to different concentrations of huCD122.

FIG. 5 shows sensorgram plots of anti-CD122 antibodies G9 (second batch) (top) and G10 (bottom) to measure binding and dissociation properties to different concentrations of huCD122.

FIG. 6 shows sensorgram plots of anti-CD122 antibodies G11 (top) and G12 (bottom) to measure binding and dissociation properties to different concentrations of huCD122.

FIG. 7 shows sensorgram plots of anti-CD122 antibodies G13 (top) and G14 (bottom) to measure binding and dissociation properties to different concentrations of huCD122.

FIG. 8 shows sensorgram plots of anti-CD122 antibodies G15 (top) and G16 (bottom) to measure binding and dissociation properties to different concentrations of huCD122.

FIG. 9 shows sensorgram plots of anti-CD122 antibodies G17 (top) and G18 (bottom) to measure binding and dissociation properties to different concentrations of huCD122.

FIG. 10 shows sensorgram plots of anti-CD122 antibodies G19 (top) and G20 (bottom) to measure binding and dissociation properties to different concentrations of huCD122.

FIG. 11 shows graphs and IC₅₀ calculations for anti-CD122 antibody inhibition of IL2 (top graph) and IL15 (bottom graph) signaling in a cell line expressing the intermediate affinity IL-βγ receptor using a reporter assay expressing luciferase under either IL2 or IL15 stimulation. Results are shown for Antibody 1, Antibody 2, and a Commercial anti-CD122 antibody.

FIG. 12 shows a graph for anti-CD122 antibody (Antibody 1 and Antibody 2) inhibition of IL2 signaling in a cell line expressing the high affinity IL-αβγ receptor using a reporter assay.

FIG. 13 shows a summary of IL2RB epitope mapping results from analyzing a humanized anti-CD122 Mab (SEQ ID NO: 187) described herein as detailed in epitope mapping experiments in Example 5.

FIG. 14 shows a heatmap of modification changes in the humanized anti-CD122 Mab bound versus unbound IL2RB as detailed in epitope mapping experiments in Example 5. Significant changes (p<0.05) are indicated by hatched boxes in the t-test column.

FIG. 15 shows a diagram of the crystal structure of Interleukin-2 receptor subunit beta (IL2RB) and IL2 with candidate epitope regions. Amino acid residues with significant changes in solvent accessibility are shown by a star (epitope region) and by arrows pointing to residues with conformational changes.

### DETAILED DESCRIPTION

Immune cell responses can be context dependent and may be influenced by signals from their environment through a variety of receptor-ligand interactions. For instance, these signals can amplify and modify a T cell receptor (TCR) signal received by antigenic stimulation in a resting naïve or memory T cell, regulate T cell proliferation and differentiation in recently activated T cells, or control effector functions in particular somatic environments. IL2 and IL15 share similar and contrasting roles in regulation of T cell function. As a non-limiting example, both IL2 and IL15 are involved in T cell differentiation. IL2 promotes the differentiation of immature T cells into regulatory T cells, which thereby are capable of suppressing other T cells that could attack normal healthy cells in the body. IL2 signaling is involved in peripheral tolerance through the elimination of self-reactive T cells by way of the activation-induced cell death (AICD) pathway. IL2 can also promote the differentiation of immature T cells into either effector T cells or into memory T cells when an initial T cell is stimulated by an antigen. IL2 has also been demonstrated to enhance the activity of both cytotoxic T cells and natural killer (NK) cells. IL15 can regulate the activation and proliferation of T cells and NK cells. In contrast to IL2 signaling, IL15 signaling can inhibit IL2-mediated AICD by eliciting antiapoptotic actions. IL15 can stimulate the persistence of memory phenotype CD8+ T cells that are involved in the elimination of invading pathogens, thereby protecting the subject against infection.

IL2 and IL15 have distinct means for initiating signaling through the various IL receptors. IL2 is secreted and can bind to heterodimeric and heterotrimeric receptors complexes both involving CD122 on the surface of activated cells. IL15 is primarily membrane bound and induces signaling in the context of cell-cell contacts, at the immunological synapse. IL15RA presents membrane bound IL15 in trans to neighboring CD8+ T cells and NK cells. Despite these differences in the mechanisms of the initiation of ligand-mediated signaling, once activated, IL2 receptors complexes and IL15 receptor complexes activate shared molecular pathways including the JAK1/JAK3/STAT5, the PI3K, and the MAPK signal transduction pathways. Activation of these pathways can modulate gene transcription to regulate apoptosis, proliferation, or differentiation of immune cells. Comprising both IL2 receptors and IL15 receptors, CD122 serves critical roles in all of these functions.

Disclosed herein, in certain aspects, are anti-CD122 antibodies, and pharmaceutical compositions which comprise the anti-CD122 antibodies. In some embodiments, also disclosed herein are methods used for targeting CD122-expressing tissues and cells with an anti-CD122 antibody described herein.

### Anti-CD122 Antibodies

Provided herein are antibodies that bind to CD122. In some instances, the antibodies that bind to CD122 are monoclonal antibodies. In certain aspects, disclosed herein is an anti-CD122 antibody. In some instances, the anti-CD122 antibody specifically binds to mammalian CD122. In some instances, the anti-CD122 antibody specifically binds to a human CD122. In some instances, the anti-CD122 antibody specifically binds to an extracellular portion of CD122. In some instances, the anti-CD122 antibody specifically binds to an extracellular portion of human CD122. In some instances, the anti-CD122 antibody is made of chimeric amino acid sequences some of which are murine-derived and some of which are human-derived. In some instances, the anti-CD122 antibody is made with complementarity-determining regions (CDRs) that have been incorporated into an antibody scaffold. In some instances, the anti-CD122 antibody is made with complementarity-determining regions (CDRs) incorporated into a human antibody variable region framework. In some instances, the human antibody variable region framework has been sequence-optimized to retain CD122 affinity with the engrafted mouse CDR sequences. In some instances, the anti-CD122 antibody is a humanized antibody. In some instances, the anti-CD122 antibody is a human antibody.

In some embodiments, the anti-CD122 antibody comprises i) a heavy chain comprising a variable heavy chain (VH) domain and ii) a light chain comprising a variable light chain (VL) domain. In some embodiments, VH domain comprises heavy chain CDR1 (HCDR1) sequence comprising a sequence selected from SEQ ID NOs: 1-11, heavy chain CDR2 (HCDR2) sequence comprising a sequence selected from SEQ ID NOs: 12-23, and heavy chain CDR3 (HCDR3) sequence comprising a sequence selected from SEQ ID NOs: 24-36. In some embodiments, VL domain comprises light chain CDR1 (LCDR1) sequence comprising a sequence selected from SEQ ID NOs: 37-47, light chain CDR2 (LCDR2) sequence comprising a sequence selected from GTS, TTS, YTS, WAS, KAS, GAT, YAS or STS, and light chain CDR3 (LCDR3) sequence comprising a sequence selected from SEQ ID NOs: 56-67.

In some embodiments, the VH region of the anti-CD122 antibody comprises HCDR1, HCDR2, and HCDR3 sequences selected from Table 1.

TABLE 1
HCDR Sequences

| SEQ ID NO: | HCDR1 Sequence |
|---|---|
| 1 | GFSLTSYG |
| 2 | GYTFTSYW |
| 3 | GSTFNRYW |
| 4 | GFTFTDYN |
| 5 | GYSFTAYT |
| 6 | GFNIKDDY |
| 7 | GYTFTSHW |
| 8 | GFTFSTFA |
| 9 | GFTFTDHT |
| 10 | GFSLTSYD |
| 11 | GYTFTAYW |
| SEQ ID NO: | HCDR2 Sequence |
| 12 | MWGGGST |
| 13 | IYPGRGST |
| 14 | ILPGSGNT |
| 15 | INPNNGRS |
| 16 | INPYNGYA |
| 17 | IDPENGDT |
| 18 | IYPGSGNT |
| 19 | ITGDGGTYT |
| 20 | IYPRDGYT |
| 21 | IWGGGGST |
| 22 | IWTGGGT |
| 23 | IDPNSGYT |
| SEQ ID NO: | HCDR3 Sequence |
| 24 | ARRTYSDSYYYEMDY |
| 25 | ARELGGFAY |
| 26 | ARLDYYGSRYYFDY |
| 27 | AREDWEGFYAMDY |
| 28 | ARVGYYFDY |
| 29 | TGYFDY |
| 30 | ARERGGFDY |
| 31 | ARHSVSSWFAY |
| 32 | ARPTSLLRFPY |
| 33 | ARHNYDGYYYSLDY |
| 34 | ARHNYDNYYYTLDY |
| 35 | VRDLFPYAMDY |
| 36 | ARGHFGYDDS |

In some embodiments, the VH region comprises HCDR1 sequence comprising SEQ ID NO: 1; HCDR2 sequence comprising SEQ ID NO: 12; and HCDR3 sequence comprising SEQ ID NO: 24. In some embodiments, the VH region comprises HCDR1 sequence comprising SEQ ID NO: 2; HCDR2 sequence comprising SEQ ID NO: 13; and HCDR3 sequence comprising SEQ ID NO: 25. In some embodiments, the VH region comprises HCDR1 sequence comprising SEQ ID NO: 3; HCDR2 sequence comprising SEQ ID NO: 14; and HCDR3 sequence comprising SEQ ID NO: 26. In some embodiments, the VH region comprises HCDR1 sequence comprising SEQ ID NO: 4; HCDR2 sequence comprising SEQ ID NO: 15; and HCDR3 sequence comprising SEQ ID NO: 27. In some embodiments, the VH region comprises HCDR1 sequence comprising SEQ ID NO: 5; HCDR2 sequence comprising SEQ ID NO: 16; and HCDR3 sequence comprising SEQ ID NO: 28. In some embodiments, the VH region comprises HCDR1 sequence comprising SEQ ID NO: 6; HCDR2 sequence comprising SEQ ID NO: 17; and HCDR3 sequence comprising SEQ ID NO: 29. In some embodiments, the VH region comprises HCDR1 sequence comprising SEQ ID NO: 7; HCDR2 sequence comprising SEQ ID NO: 18; and HCDR3 sequence comprising SEQ ID NO: 30. In some embodiments, the VH region comprises HCDR1 sequence comprising SEQ ID NO: 8; HCDR2 sequence comprising SEQ ID NO: 19; and HCDR3 sequence comprising SEQ ID NO: 31. In some embodiments, the VH region comprises HCDR1 sequence comprising SEQ ID NO: 9; HCDR2 sequence comprising SEQ ID NO: 20; and HCDR3 sequence comprising SEQ ID NO: 32. In some embodiments, the VH region comprises HCDR1 sequence comprising SEQ ID NO: 1; HCDR2 sequence comprising SEQ ID NO: 21; and HCDR3 sequence comprising SEQ ID NO: 33. In some embodiments, the VH region comprises HCDR1 sequence comprising SEQ ID NO: 1; HCDR2 sequence comprising SEQ ID NO: 21; and HCDR3 sequence comprising SEQ ID NO: 34. In some embodiments, the VH region comprises HCDR1 sequence comprising SEQ ID NO: 10; HCDR2 sequence comprising SEQ ID NO: 22; and HCDR3 sequence comprising SEQ ID NO: 35. In some embodiments, the VH region comprises HCDR1 sequence comprising SEQ ID NO: 11; HCDR2 sequence comprising SEQ ID NO: 23; and HCDR3 sequence comprising SEQ ID NO: 36.

In some embodiments, the VL region of the anti-CD122 antibody comprises LCDR1, LCDR2, and LCDR3 sequences selected from Table 2.

TABLE 2
LCDR Sequences

| SEQ ID NO: | LCDR1 Sequence |
|---|---|
| 37 | SSVSSRY |
| 38 | SYVSSSY |
| 39 | QDIRNY |
| 40 | QNVDTD |
| 41 | SSVSY |
| 42 | QNINLW |
| 43 | QDISNF |
| 44 | SSVNY |
| 45 | EDIDSY |
| 46 | QTIGTS |
| 47 | SRVSY |
| SEQ ID NO: | LCDR2 Sequence |
| | GTS |
| | TTS |
| | YTS |
| | WAS |
| | KAS |
| | GAT |
| | YAS |
| | STS |
| SEQ ID NO: | LCDR3 Sequence |
| 56 | QQYHSDPLT |
| 57 | HQYHLSPWT |
| 58 | QQGDPLPPT |
| 59 | EQYSSYPYT |
| 60 | QQFTSSPYT |
| 61 | LQGQSYPWT |
| 62 | QQDNNHPYT |
| 63 | QQFTSSPWT |
| 64 | QHYYSIPYT |
| 65 | QQYHGDPLT |
| 66 | QQTNSWPLT |
| 67 | QQRSSYPLT |

In some embodiments, the VL region comprises LCDR1 sequence comprising SEQ ID NO: 37; LCDR2 sequence comprising SEQ ID NO: GTS; and LCDR3 sequence comprising SEQ ID NO: 56. In some embodiments, the VL region comprises LCDR1 sequence comprising SEQ ID NO: 38; LCDR2 sequence comprising SEQ ID NO: TTS; and LCDR3 sequence comprising SEQ ID NO: 57. In some embodiments, the VL region comprises LCDR1 sequence comprising SEQ ID NO: 39; LCDR2 sequence comprising SEQ ID NO: YTS; and LCDR3 sequence comprising SEQ ID NO: 58. In some embodiments, the VL region comprises LCDR1 sequence comprising SEQ ID NO: 40; LCDR2 sequence comprising SEQ ID NO: WAS; and LCDR3 sequence comprising SEQ ID NO: 59. In some embodiments, the VL region comprises LCDR1 sequence comprising SEQ ID NO: 41; LCDR2 sequence comprising SEQ ID NO: YTS; and LCDR3 sequence comprising SEQ ID NO: 60. In some embodiments, the VL region comprises LCDR1 sequence comprising SEQ ID NO: 42; LCDR2 sequence comprising KAS; and LCDR3 sequence comprising SEQ ID NO: 61. In some embodiments, the VL region comprises LCDR1 sequence comprising SEQ ID NO: 43; LCDR2 sequence comprising YTS; and LCDR3 sequence comprising SEQ ID NO: 62. In some embodiments, the VL region comprises LCDR1 sequence comprising SEQ ID NO: 44; LCDR2 sequence comprising YTS; and LCDR3 sequence comprising SEQ ID NO: 63. In some embodiments, the VL region comprises LCDR1 sequence comprising SEQ ID NO: 45; LCDR2 sequence comprising GAT; and LCDR3 sequence comprising SEQ ID NO: 64. In some embodiments, the VL region comprises LCDR1 sequence comprising SEQ ID NO: 37; LCDR2 sequence comprising GTS; and LCDR3 sequence comprising SEQ ID NO: 65. In some embodiments, the VL region comprises LCDR1 sequence comprising SEQ ID NO: 46; LCDR2 sequence comprising YAS; and LCDR3 sequence comprising SEQ ID NO: 66. In some embodiments, the VL region comprises LCDR1 sequence comprising SEQ ID NO: 47; LCDR2 sequence comprising STS; and LCDR3 sequence comprising SEQ ID NO: 67.

In some embodiments, the anti-CD122 antibody comprises a VH region and a VL region, in which the VH region comprises HCDR1 sequence comprising SEQ ID NO: 1; HCDR2 sequence comprising SEQ ID NO: 12; and HCDR3 sequence comprising SEQ ID NO: 24 and in which the VL region comprises LCDR1 sequence comprising SEQ ID NO: 37; LCDR2 sequence comprising GTS; and LCDR3 sequence comprising SEQ ID NO: 56. In some embodiments, the anti-CD122 antibody comprises a VH region and a VL region, in which the VH region comprises HCDR1 sequence comprising SEQ ID NO: 2; HCDR2 sequence comprising SEQ ID NO: 13; and HCDR3 sequence comprising SEQ ID NO: 25 and in which the VL region comprises LCDR1 sequence comprising SEQ ID NO: 38; LCDR2 sequence comprising TTS; and LCDR3 sequence comprising SEQ ID NO: 57. In some embodiments, the anti-CD122 antibody comprises a VH region and a VL region, in which the VH region comprises HCDR1 sequence comprising SEQ ID NO: 3; HCDR2 sequence comprising SEQ ID NO: 14; and HCDR3 sequence comprising SEQ ID NO: 26 and in which the VL region comprises LCDR1 sequence comprising SEQ ID NO: 39; LCDR2 sequence comprising YTS; and LCDR3 sequence comprising SEQ ID NO: 58. In some embodiments, the anti-CD122 antibody comprises a VH region and a VL region, in which the VH region comprises HCDR1 sequence comprising SEQ ID NO: 4; HCDR2 sequence comprising SEQ ID NO: 15; and HCDR3 sequence comprising SEQ ID NO: 27 and in which the VL region LCDR1 sequence comprising SEQ ID NO: 40; LCDR2 sequence comprising WAS; and LCDR3 sequence comprising SEQ ID NO: 59. In some embodiments, the anti-CD122 antibody comprises a VH region and a VL region, in which the VH region comprises HCDR1 sequence comprising SEQ ID NO: 5; HCDR2 sequence comprising SEQ ID NO: 16; and HCDR3 sequence comprising SEQ ID NO: 28 and in which the VL region comprises LCDR1 sequence comprising SEQ ID NO: 41; LCDR2 sequence comprising YTS; and LCDR3 sequence comprising SEQ ID NO: 60. In some embodiments, the anti-CD122 antibody comprises a VH region and a VL region, in which the VH region comprises HCDR1 sequence comprising SEQ ID NO: 6; HCDR2 sequence comprising SEQ ID NO: 17; and HCDR3 sequence comprising SEQ ID NO: 29 and in which the VL region comprises LCDR1 sequence comprising SEQ ID NO: 42; LCDR2 sequence comprising KAS; and LCDR3 sequence comprising SEQ ID NO: 61. In some embodiments, the anti-CD122 antibody comprises a VH region and a VL region, in which the VH region comprises HCDR1 sequence comprising SEQ ID NO: 7; HCDR2 sequence comprising SEQ ID NO: 18; and HCDR3 sequence comprising SEQ ID NO: 30 and in which the VL region comprises LCDR1 sequence comprising SEQ ID NO: 43; LCDR2 sequence comprising YTS; and LCDR3 sequence comprising SEQ ID NO: 62. In some embodiments, the anti-CD122 antibody comprises a VH region and a VL region, in which the VH region comprises HCDR1 sequence comprising SEQ ID NO: 8; HCDR2 sequence comprising SEQ ID NO: 19; and HCDR3 sequence comprising SEQ ID NO: 31 and in which the VL region comprises LCDR1 sequence comprising SEQ ID NO: 44; LCDR2 sequence comprising YTS; and LCDR3 sequence comprising SEQ ID NO: 63. In some embodiments, the anti-CD122 antibody comprises a VH region and a VL region, in which the VH region comprises HCDR1 sequence comprising SEQ ID NO: 9; HCDR2 sequence comprising SEQ ID NO: 20; and HCDR3 sequence comprising SEQ ID NO: 32 and in which the VL region comprises LCDR1 sequence comprising SEQ ID NO: 45; LCDR2 sequence comprising GAT; and LCDR3 sequence comprising SEQ ID NO: 64. In some embodiments, the anti-CD122 antibody comprises a VH region and a VL region, in which the VH region comprises HCDR1 sequence comprising SEQ ID NO: 1; HCDR2 sequence comprising SEQ ID NO: 21; and HCDR3 sequence comprising SEQ ID NO: 33 and in which the VL region comprises LCDR1 sequence comprising SEQ ID NO: 37; LCDR2 sequence comprising GTS; and LCDR3 sequence comprising SEQ ID NO: 65. In some embodiments, the anti-CD122 antibody comprises a VH region and a VL region, in which the VH region comprises HCDR1 sequence comprising SEQ ID NO: 1; HCDR2 sequence comprising SEQ ID NO: 21; and HCDR3 sequence comprising SEQ ID NO: 34 and in which the VL region comprises LCDR1 sequence comprising SEQ ID NO: 37; LCDR2 sequence comprising GTS; and LCDR3 sequence comprising SEQ ID NO: 65. In some embodiments, the anti-CD122 antibody comprises a VH region and a VL region, in which the VH region comprises HCDR1 sequence comprising SEQ ID NO: 10; HCDR2 sequence comprising SEQ ID NO: 22; and HCDR3 sequence comprising SEQ ID NO: 35 and in which the VL region comprises LCDR1 sequence comprising SEQ ID NO: 46; LCDR2 sequence comprising YAS; and LCDR3 sequence comprising SEQ ID NO: 66. In some embodiments, the anti-CD122 antibody comprises a VH region and a VL region, in which the VH region comprises HCDR1 sequence comprising SEQ ID NO: 11; HCDR2 sequence comprising SEQ ID NO: 23; and HCDR3 sequence comprising SEQ ID NO: 36 and in which the VL region comprises LCDR1 sequence comprising SEQ ID NO: 47; LCDR2 sequence comprising STS; and LCDR3 sequence comprising SEQ ID NO: 67.

In some embodiments, the anti-CD122 antibody comprises a series of CDR sequences. In some embodiments, the series of CDR sequences comprises an HCDR1 sequence, an HCDR2 sequence, an HCDR3 sequence, an LCDR1 sequence, an LCDR2 sequence, and an LCDR3 sequence. The series of CDR sequences for each anti-CD122 antibody described herein is listed in Table 3. The names for the series of CDR sequences are E1-E13.

TABLE 3
Series of CDR sequences for anti-CD122 antibodies

| Name | HCDR1 | HCDR2 | HCDR3 | LCDR1 | LCDR2 | LCDR3 |
|---|---|---|---|---|---|---|
| E1 | SEQ ID | SEQ ID | SEQ ID | SEQ ID | GTS | SEQ ID |
| | NO: 1 | NO: 12 | NO: 24 | NO: 37 | | NO: 56 |
| E2 | SEQ ID | SEQ ID | SEQ ID | SEQ ID | TTS | SEQ ID |
| | NO: 2 | NO: 13 | NO: 25 | NO: 38 | | NO: 57 |
| E3 | SEQ ID | SEQ ID | SEQ ID | SEQ ID | YTS | SEQ ID |
| | NO: 3 | NO: 14 | NO: 26 | NO: 39 | | NO: 58 |
| E4 | SEQ ID | SEQ ID | SEQ ID | SEQ ID | WAS | SEQ ID |
| | NO: 4 | NO: 15 | NO: 27 | NO: 40 | | NO: 59 |
| ES | SEQ ID | SEQ ID | SEQ ID | SEQ ID | YTS | SEQ ID |
| | NO: 5 | NO: 16 | NO: 28 | NO: 41 | | NO: 60 |
| E6 | SEQ ID | SEQ ID | SEQ ID | SEQ ID | KAS | SEQ ID |
| | NO: 6 | NO: 17 | NO: 29 | NO: 42 | | NO: 61 |
| E7 | SEQ ID | SEQ ID | SEQ ID | SEQ ID | YTS | SEQ ID |
| | NO: 7 | NO: 18 | NO: 30 | NO: 43 | | NO: 62 |
| E8 | SEQ ID | SEQ ID | SEQ ID | SEQ ID | YTS | SEQ ID |
| | NO: 8 | NO: 19 | NO: 31 | NO: 44 | | NO: 63 |
| E9 | SEQ ID | SEQ ID | SEQ ID | SEQ ID | GAT | SEQ ID |
| | NO: 9 | NO: 20 | NO: 32 | NO: 45 | | NO: 64 |
| E10 | SEQ ID | SEQ ID | SEQ ID | SEQ ID | GTS | SEQ ID |
| | NO: 1 | NO: 21 | NO: 33 | NO: 37 | | NO: 65 |
| E11 | SEQ ID | SEQ ID | SEQ ID | SEQ ID | GTS | SEQ ID |
| | NO: 1 | NO: 21 | NO: 34 | NO: 37 | | NO: 65 |
| E12 | SEQ ID | SEQ ID | SEQ ID | SEQ ID | YAS | SEQ ID |
| | NO: 10 | NO: 22 | NO: 35 | NO: 46 | | NO: 66 |
| E13 | SEQ ID | SEQ ID | SEQ ID | SEQ ID | STS | SEQ ID |
| | NO: 11 | NO: 23 | NO: 36 | NO: 47 | | NO: 67 |

In some embodiments, the anti-CD122 antibody comprises a framework for grafting CDRs from another animal species. In some embodiments, CDRs from a mammalian antibody are grafted onto a human framework sequence. In some embodiments, CDRs from a mouse antibody are grafted onto a human framework sequence. In some embodiments, the human framework sequence forms part of the VH region of an antibody. In some embodiments, sequences from HCDR1, HCDR2, and HCDR3 are grafted into a human framework sequence.

In some embodiments, an anti-CD122 antibody described herein comprises an IgG framework, an IgA framework, an IgE framework, or an IgM framework. In some instances, the anti-CD122 antibody comprises an IgG framework (e.g., IgG1, IgG2, IgG3, or IgG4). In some cases, the anti-CD122 antibody comprises an IgG1 framework. In some cases, the anti-CD122 antibody comprises an IgG2 (e.g., an IgG2a or IgG2b) framework. In some cases, the anti-CD122 antibody comprises an IgG2a framework. In some cases, the anti-CD122 antibody comprises an IgG2b framework. In some cases, the anti-CD122 antibody comprises an IgG3 framework. In some cases, the anti-CD122 antibody comprises an IgG4 framework. In some embodiments, the human framework sequence is inserted into an IgG backbone to form a heavy chain sequence. In some embodiments, the IgG backbone is an IgG1 backbone, an IgG2 backbone, an IgG3 backbone, or an IgG4 backbone. In some embodiments, the IgG framework comprises a human IgG heavy chain framework sequence. In some embodiments, the human IgG heavy chain framework sequence has anti-CD122 HCDR sequences grafted into it. In some embodiments, the human IgG heavy chain framework sequence with anti-CD122 HCDR sequences grafted into it is analyzed and modeled for monoclonal antibody 3D structure to identify key amino acid positions supporting CDR loop structure. In some embodiments, key amino acid positions are identified in the human IgG heavy chain framework sequence that if reverted back to a mouse framework sequence from a mouse anti-CD122 antibody, will restore affinity to human CD122 in the context of a humanized antibody using a human IgG heavy chain framework sequence that has mouse anti-CD122 CDR sequences grafted into it. In some embodiments, those key amino acid positions are mutated in the human IgG heavy chain framework sequences back to the mouse sequences and are called back mutations. In some embodiments, the human IgG heavy chain framework sequences with mouse anti-CD122 CDR sequences grafted into it and back mutations incorporated into the framework sequence are used in a human IgG backbone to create a humanized IgG heavy chain sequence. In some embodiments, the humanized IgG heavy chain sequence is used to create an anti-CD122 antibody. Table 4 lists human IgG heavy chain framework sequences used herein for grafting anti-CD122 HCDR sequences. Table 4 also lists human IgG heavy chain framework sequences used herein for grafting anti-CD122 HCDR sequences that have had back mutations incorporated into the framework to potentially restore an affinity lost to the human CD122 ligand. Table 4 also lists a mouse parental heavy chain variable framework sequence used to incorporate the mouse anti-CD122 heavy chain variable regions into a human IgG backbone to create an anti-CD122 chimera heavy chain. In Table 4 sequences disclosed, X residues are the CDR insertion sites. In Table 4 sequences disclosed, (X)ₙ₌₁₋₂₅ refers to a CDR1, a CDR2, or a CDR3 sequence wherein X refers to a CDR amino acid sequence and n equals the number of amino acid residues in a CDR sequence. In Table 4, the CDR1, CDR2, or CDR3 sequences are HCDR sequences. The framework sequences disclosed in Table 4 are given names F1-F7. In some embodiments, F1 is a human IgG heavy chain framework sequence used herein for grafting anti-CD122 HCDR sequences. In some embodiments, F2 is a human IgG heavy chain framework sequence used herein for grafting anti-CD122 HCDR sequences. In some embodiments, F3 is a human IgG heavy chain framework sequence used herein for grafting anti-CD122 HCDR sequences. In some embodiments, F4 is a human IgG heavy chain framework sequence used herein for grafting anti-CD122 HCDR sequence that have had back mutations incorporated into the framework to potentially restore an affinity lost to the human CD122 ligand. In some embodiments, F5 is a human IgG heavy chain framework sequence used herein for grafting anti-CD122 HCDR sequences that have had back mutations incorporated into the framework to potentially restore an affinity lost to the human CD122 ligand. In some embodiments, F6 is a human IgG heavy chain framework sequence used herein for grafting anti-CD122 HCDR sequences that have had back mutations incorporated into the framework to potentially restore an affinity lost to the human CD122 ligand. In some embodiments, F7 is a mouse parental heavy chain variable framework sequence used to incorporate mouse anti-CD122 heavy chain variable regions into a human IgG backbone to create an anti-CD122 chimera heavy chain into the framework to potentially restore an affinity lost to the human CD122 ligand. In some embodiments, HCDR sequences from one of series E1-E13 are grafted into F1 to create a heavy chain variable domain sequence. In some embodiments, HCDR sequences from one of series E1-E13 are grafted into F2 to create a heavy chain variable domain sequence. In some embodiments, HCDR sequences from one of series E1-E13 are grafted into F3 to create a heavy chain variable domain sequence. In some embodiments, HCDR sequences from one of series E1-E13 are grafted into F4 to create a heavy chain variable domain sequence. In some embodiments, HCDR sequences from one of series E1-E13 are grafted into F5 to create a heavy chain variable domain sequence. In some embodiments, HCDR sequences from one of series E1-E13 are grafted into F6 to create a heavy chain variable domain sequence. In some embodiments, HCDR sequences from one of series E1-E13 are grafted into F7 to create a heavy chain variable domain sequence.

TABLE 4
human IgG heavy chain framework sequences and
parental mouse IgG heavy chain framework sequences

| | SEQ | |
|---|---|---|
| | ID | |
| Name | NO: | Framework Sequence |
| F1 | 68 | QVQLVQSGAEVKKPGASVKVSCKAS(X)ₙ ₌ ₁₋₂₅ |
| | | ISWVRQAPGQGLEWMGW(X)ₙ ₌ ₁₋₂₅ |
| | | NYAQKLQGRVTMTTDTSTSTAYMELRSLRSDDTAVYYC(X)ₙ ₌ ₁₋₂₅ |
| | | WGQGTLVTVSS |
| F2 | 69 | QVQLVQSGAEVKKPGASVKVSCKAS(X)ₙ ₌ ₁₋₂₅ |
| | | MHWVRQAPGQGLEWMGR(X)ₙ ₌ ₁₋₂₅ |
| | | NYAQKFQGRVTSTRDTSISTAYMELSRLRSDDTVVYYC(X)ₙ ₌ ₁₋₂₅ |
| | | WGQGTLVTVSS |
| F3 | 70 | QVQLVQSGAEVKKPGASVKVSCKAS(X)ₙ ₌ ₁₋₂₅ |
| | | MHWVRQAPGQRLEWMGW(X)ₙ ₌ ₁₋₂₅ |
| | | KYSQKFQGRVTITRDTSASTAYMELSSLRSEDTAVYYC(X)ₙ ₌ ₁₋₂₅ |
| | | WGQGTLVTVSS |
| F4 | 71 | QVQLVQSGAEVKKPGASVKVSCKAS(X)ₙ ₌ ₁₋₂₅ |
| | | ISWVRQAPGQGLEWMGD(X)ₙ ₌ ₁₋₂₅ |
| | | NYNEKLQGRVTMTVDTSTSTAYMELRSLRSDDTAVYYC(X)ₙ ₌ ₁₋₂₅ |
| | | WGQGTLVTVSS |
| F5 | 72 | QVQLVQSGAEVKKPGASVKVSCKAS(X)ₙ ₌ ₁₋₂₅ |
| | | ITWVRQAPGQGLEWMGD(X)ₙ ₌ ₁₋₂₅ |
| | | NYNEKFQGRVTLTVDTSISTAYMELSRLRSDDTVVYYC(X)ₙ ₌ ₁₋₂₅ |
| | | WGQGTLVTVSS |
| F6 | 73 | QVQLVQSGAEVKKPGASVKVSCKAS(X)ₙ ₌ ₁₋₂₅ |
| | | ITWVRQAPGQRLEWMGD(X)ₙ ₌ ₁₋₂₅ |
| | | NYNEKFQGRVTITVDTSASTAYMELSSLRSEDTAVYYC(X)ₙ ₌ ₁₋₂₅ |
| | | WGQGTLVTVSS |
| F7 | 74 | QVQLQQPGAELVKPGASVKMSCKAS(X)ₙ ₌ ₁₋₂₅ |
| | | ITWVKQRPGQGLEWIGD(X)ₙ ₌ ₁₋₂₅ |
| | | NYNEKFKSKATLTVDTSSSTAYMQLSSLTSEDSADYYC(X)ₙ ₌ ₁₋₂₅ |
| | | WGQGTTLTVSS |

(X)_{n =} ₁₋₂₅ is a CDR1, a CDR2, or a CDR3 sequence.

In some embodiments, the anti-CD122 antibody comprises a framework for grafting CDRs from another animal species. In some embodiments, CDRs from a mammalian antibody are grafted onto a human framework sequence. In some embodiments, CDRs from a mouse antibody are grafted onto a human framework sequence. In some embodiments, the human framework sequence forms part of the VL region of an antibody. In some embodiments, sequences from LCDR1, LCDR2, and LCDR3 are grafted into a human framework sequence.

In some embodiments, an anti-CD122 antibody described herein comprises a lambda or a kappa framework. In some instances, the anti-CD122 antibody comprises a kappa framework that has LCDR1, LCDR2, and LCDR3 sequences grafted into it. In some instances, the anti-CD122 antibody comprises a kappa framework that has LCDR1, LCDR2, and LCDR3 sequences grafted into it. In some embodiments, the kappa framework forms part of a light chain. In some embodiments, the kappa framework forming part of a light chain is paired with a heavy chain described herein. In some embodiments, the kappa framework is a human kappa light chain framework. In some embodiments, the human kappa light chain framework comprises a human kappa light chain framework sequence. In some embodiments, the human kappa light chain framework sequence has anti-CD122 LCDR sequences grafted into it. In some embodiments, the human kappa light chain framework sequence with anti-CD122 LCDR sequences grafted into it is analyzed and modeled for monoclonal antibody 3D structure to identify key amino acid positions supporting CDR loop structure. In some embodiments, key amino acid positions are identified in the human kappa light chain framework sequence that if reverted back to a mouse framework sequence from a mouse anti-CD122 antibody, will restore affinity to human CD122 in the context of a humanized antibody using a human kappa light chain framework sequence that has mouse anti-CD122 CDR sequences grafted into it. In some embodiments, those key amino acid positions are mutated in the human kappa light chain framework sequences back to the mouse sequences and are called back mutations. In some embodiments, the human kappa light chain framework sequences with mouse anti-CD122 CDR sequences grafted into it and back mutations incorporated into the framework sequence are used in a human light chain backbone to create a humanized light chain sequence. In some embodiments, the humanized kappa light chain sequence is used to create an anti-CD122 antibody. Table 5 lists human kappa light chain framework sequences used herein for grafting anti-CD122 LCDR sequences. Table 5 also lists human kappa light chain framework sequences used herein for grafting anti-CD122 LCDR sequences that have had back mutations incorporated into the framework to potentially restore an affinity lost to the human CD122 ligand. Table 5 also lists the mouse parental light chain variable framework sequence used to incorporate the mouse anti-CD122 light chain variable regions into a human light chain backbone to create an anti-CD122 chimera light chain. In the Table 5 sequences disclosed, X residues are the CDR insertion sites. In Table 5 sequences disclosed, (X)ₙ₌₁₋₂₅ refers to a CDR1, a CDR2, or a CDR3 sequence wherein X refers to a CDR amino acid sequence and n equals the number of amino acid residues in a CDR sequence. In Table 5, the CDR1, CDR2, or CDR3 sequences are LCDR sequences. The framework sequences disclosed in Table 5 are given names F8-F14. In some embodiments, F8 is a human kappa light chain framework sequence used herein for grafting anti-CD122 LCDR sequences. In some embodiments, F9 is a human kappa light chain framework sequence used herein for grafting anti-CD122 LCDR sequences. In some embodiments, F10 is a human kappa light chain framework sequence used herein for grafting anti-CD122 LCDR sequences. In some embodiments, F11 is a human kappa light chain framework sequence used herein for grafting anti-CD122 LCDR sequences that have had back mutations incorporated into the framework to potentially restore an affinity lost to the human CD122 ligand. In some embodiments, F12 is a human kappa light chain framework sequence used herein for grafting anti-CD122 LCDR sequences that have had back mutations incorporated into the framework to potentially restore an affinity lost to the human CD122 ligand. In some embodiments, F13 is a human IgG heavy chain framework sequence used herein for grafting anti-CD122 LCDR sequences that have had back mutations incorporated into the framework to potentially restore an affinity lost to the human CD122 ligand. In some embodiments, F14 is a mouse parental light chain variable framework sequence used to incorporate mouse anti-CD122 light chain variable regions into a human light chain backbone to create an anti-CD122 chimera light chain into the framework to potentially restore an affinity lost to the human CD122 ligand. In some embodiments, LCDR sequences from one of series E1-E13 are grafted into F8 to create a light chain variable domain sequence. In some embodiments, LCDR sequences from one of series E1-E13 are grafted into F9 to create a light chain variable domain sequence. In some embodiments, LCDR sequences from one of series E1-E13 are grafted into F10 to create a light chain variable domain sequence. In some embodiments, LCDR sequences from one of series E1-E13 are grafted into F11 to create a light chain variable domain sequence. In some embodiments, LCDR sequences from one of series E1-E13 are grafted into F12 to create a light chain variable domain sequence. In some embodiments, LCDR sequences from one of series E1-E13 are grafted into F13 to create a light chain variable domain sequence. In some embodiments, LCDR sequences from one of series E1-E13 are grafted into F14 to create a light chain variable domain sequence.

TABLE 5
human kappa light chain framework sequences and parental
mouse light chain framework sequences

| Name | SEQ ID NO: | Framework Sequence |
|---|---|---|
| F8 | 75 | DIQMTQSPSSLSASVGDRVTITCRAS(X)_{n = 1-25} |
| | | LAWYQQKPGKVPKLLIY(X)_{n = 1-25} |
| | | TLQSGVPSRFSGSGSGTDFTLTISSLQPEDVATYYC(X)_{n = 1-25} |
| | | FGQGTKLEIK |
| F9 | 76 | DIQMTQSPSSLSASVGDRVTITCQAS(X)_{n = 1-25} |
| | | LNWYQQKPGKAPKLLIY(X)_{n = 1-25} |
| | | NLETGVPSRFSGSGSGTDFTFTISSLQPEDIATYYC(X)_{n = 1-25} |
| | | FGQGTKLEIK |
| F10 | 77 | DIQMTQSPSSLSASVGDRVTITCRAS(X)_{n = 1-25} |
| | | LNWYQQKPGKAPKLLIY(X)_{n = 1-25} |
| | | SLQSGVPSRFSGSGSGTDFTLTISSLQPEDFATYYC(X)_{n = 1-25} |
| | | FGQGTKLEIK |
| F11 | 78 | DIQMTQSPSSLSASVGDRVTITCRAS(X)_{n = 1-25} |
| | | LNWYQQKPEGTLKLLIY(X)_{n = 1-25} |
| | | GLHSGVPSRFSGSGSGTDFTLTISSLQPEDVATYYC(X)_{n = 1-25} |
| | | FGQGTKLEIK |
| F12 | 79 | DIQMTQSPSSLSASVGDRVTITCQAS(X)_{n = 1-25} |
| | | LNWYQQKPGKTLKLLIY(X)_{n = 1-25} |
| | | GLHTGVPSRFSGSGSGTDFTFTISSLQPEDIATYYC(X)_{n = 1-25} |
| | | FGQGTKLEIK |
| F13 | 80 | DIQMTQSPSSLSASVGDRVTITCRAS(X)_{n = 1-25} |
| | | LNWYQQKPGKAPKLLIY(X)_{n = 1-25} |
| | | GLHSGVPSRFSGSGSGTDFTLTISSLQPEDFATYYC(X)_{n = 1-25} |
| | | FGQGTKLEIK |
| F14 | 81 | DIQMTQTTSSLSASLGDRVTINCRAS(X)_{n = 1-25} |
| | | LNWYQQKPDGTLKLLIY(X)_{n = 1-25} |
| | | GLHSGVPSRFSGSGSGTDYSLTISNLEEEDIATYFC(X)_{n = 1-25} |
| | | FGSGTKLEIK |

(X)_{n = 1-25} is CDR1, CDR2, or CDR3 sequence

In some embodiments, the anti-CD122 antibody comprises a VH region and a VL region in which the sequence of the VH region comprises about 80%, 85%, 90%, 95%, 96% 97%, 98%, 99%, or 100% sequence identity to SEQ ID NOs: 82-94 and the sequence of the VL region comprises about 80%, 85%, 90%, 95%, 96% 97%, 98%, 99%, or 100% sequence identity to SEQ ID NOs: 95-107. In some embodiments, the anti-CD122 antibody comprises the VH domain comprising at least 80% sequence identity to a sequence selected from SEQ ID NOS: 82-94. In some embodiments, the anti-CD122 antibody comprises the VH domain comprising at least 85% sequence identity to a sequence selected from SEQ ID NOs: 82-94. In some embodiments, the anti-CD122 antibody comprises the VH domain comprising at least 90% sequence identity to a sequence selected from SEQ ID NOs: 82-94. In some embodiments, the anti-CD122 antibody comprises the VH domain comprising at least 95% sequence identity to a sequence selected from SEQ ID NOs: 82-94. In some embodiments, the anti-CD122 antibody comprises the VL domain comprising at least 80% sequence identity to a sequence selected from SEQ ID NOs: 95-107. In some embodiments, the anti-CD122 antibody comprises the VL domain comprising at least 85% sequence identity to a sequence selected from SEQ ID NOs: 95-107. In some embodiments, the anti-CD122 antibody comprises the VL domain comprising at least 90% sequence identity to a sequence selected from SEQ ID NOs: 95-107. In some embodiments, the anti-CD122 antibody comprises the VL domain comprising at least 95% sequence identity to a sequence selected from SEQ ID NOs: 95-107. In some embodiments, the VH region comprises a sequence selected from SEQ ID NOs: 82-94 (Table 6) and the VL region comprises a sequence selected from SEQ ID NOs: 95-107 (Table 7). VH sequences, names and corresponding SEQ ID NOs are listed in Table 6. VL sequences, names and corresponding SEQ ID NOs are listed in Table 7.

TABLE 6
VH Sequences

| SEQ ID | |
|---|---|
| NO: | VH Sequence |
| 82 | QVQLKESGPGLVAPSQSLSITCTVSGFSLTSYGIDWVRQPPGKGLEWLGIMWGGG |
| | STNYNSALMSRLSISKDISKSQVFLKVNSLQTDDTAMYYCARRTYSDSYYYEMD |
| | YWGQGTSVTVSS |
| 83 | QVQLQQPGTELVKPGASVKMSCKASGYTFTSYWITWVKQRPGQGLEWIGDIYPG |
| | RGSTNYNEKFKSKATLTVDTSSSTAYMQLSSLTSEDSAVYYCARELGGFAYWGQ |
| | GTLVTVSA |
| 84 | QVQLQQSGAELMKPGASVKISCKATGSTFNRYWIEWVKQRPGHGLEWIGEILPGS |
| | GNTNYNEKFKGKATFTADTSSNTAYMQLSSLTSEDSAVYYCARLDYYGSRYYFD |
| | YWGQGTTLTVSS |
| 85 | EVQLQQSGPELVKPGASVKMSCKASGFTFTDYNIHWVKQSHGKSLEWIGYINPN |
| | NGRSSYNLYFKDKATLTVNKSSSTAYMELRSLTSEDSAVYYCAREDWEGFYAM |
| | DYWGQGTSVTVSS |
| 86 | EVQLQQSGPELVKPGASMKISCKASGYSFTAYTMNWVRQSHGKNLEWIGLINPY |
| | NGYANYNQKFKGKATLTVDKSSSTAYMDLLSLTSEDSAVYYCARVGYYFDYWG |
| | QGTTLTVSS |
| 87 | EVQLQQSGAELVRPGASVKLSCTSSGFNIKDDYMHWVKQRPEQGLEWIGWIDPE |
| | NGDTEYASKFQGKATITADTSSNTAYLQLNSLTSEDTAVYYCTGYFDYWGQGTT |
| | LTVSS |
| 88 | QVQLQQPGAELVKPGASVKMSCKASGYTFTSHWITWVKQRPGQGLEWIGDIYPG |
| | SGNTNYNEKFKSKATLTVDTSSSTAYMQLSSLTSEDSADYYCARERGGFDYWGQ |
| | GTTLTVSS |
| 89 | DVILVESGGDLVKPGGSLKLSCAASGFTFSTFAMSWVRQTPEKRLEWVASITGDG |
| | GTYTYYSDSVKGRFTISRDNARNTLYLQMSSLRSEDTAFYYCARHSVSSWFAYW |
| | GQGTLVTVSA |
| 90 | QVQLLQSDAELVKPGASVKISCKVSGFTFTDHTLHWMKQRPEQGLEWIGYIYPR |
| | DGYTKYNEKFKGKATLTADKSSSTAYMQLDSLTSEDSAVYFCARPTSLLRFPYW |
| | GQGTLVTVSA |
| 91 | QVQLKESGPGLVAPSQSLSITCTVSGFSLTSYGVDWVRQPPGKGLEWLGVIWGG |
| | GGSTNYNSALMSRLSISKDNSKSQVFLKMNSLQTDDTAMYYCARHNYDGYYYS |
| | LDYWGQGTSVTVSS |
| 92 | QVQLKESGPGLVAPSQSLSITCTVSGFSLTSYGVDWVRQPPGKGLEWLGVIWGG |
| | GGSTNYNSALMSRLSISKDNSKSQVFLKMNSLQTDDTAMYYCARHNYDNYYYT |
| | LDYWGQGTSVTVSS |
| 93 | QVQLKESGPGLVAPSQSLSITCTVSGFSLTSYDISWIRQPPGKGLEWLGVIWTG |
| | GGTNYNSPFMSRLTISKDNSRSQVFLKMNSLQTDDTAMYYCVRDLFPYAMDYWG |
| | QGTSVTVSS |
| 94 | QVQLQQSGAELAKPGASVKMSCKASGYTFTAYWIHWVKQRPGQGLEWIGYIDPN |
| | SGYTDYNQIFKDKATLTADRSSSTAYMQLNSLTSEDSAVYYCARGHFGYDDSW |
| | GQGITLTVSS |

TABLE 7
VL Sequences

| SEQ ID | |
|---|---|
| NO: | VL Sequence |
| 95 | QIVLTQSPAIMSASPGEKVTMTCSASSSVSSRYLHWYQQKSGASPKLWIYGTSN |
| | LASGVPARFSGSGSGTSYSLTISSVEAEDAATYYCQQYHSDPLTFGAGTKLELK |
| 96 | QIVLTQSPAIMSASLGERVTMTCTASSYVSSSYLHWYQQKPGSSPTLWIYTTSN |
| | LASGVPARFSGSGSGTSYSLTISNMEAEDAATYYCHQYHLSPWTFGGGTKLEIK |
| 97 | DIQMTQTTSSLSASLGDRVTISCRASQDIRNYLNWYQQKPDGTLKLLIYYTSRL |
| | HSGVPSRFSGSGSGTDYSLTISNLEQEDVATYFCQQGDPLPPTFGGGTKVEIK |
| 98 | DIVMTQSQKFMSTSVGDRVSITCKASQNVDTDVSWYQQKPGKSPKTLIYWASN |
| | RFTGVPDRFTGSGSGTDFTLTITNVQSEDLADYFCEQYSSYPYTFGSGTKLEIK |
| 99 | ENVLTQSPTIMSASLGEKVTMSCRASSSVSYMHWYQQKSDASPKLWIYYTSNL |
| | APGVPARFSASGSGNSYSLTISSMEGEDAATYYCQQFTSSPYTFGGGTKLEIK |
| 100 | DIQMNQSPSSLSASLGDTISISCRASQNINLWLNWYQQKPGNVPKLLIFKASNL |
| | HPGVPSRFSGSGSGTDFTLTISSLQPEDIATYYCLQGQSYPWTFGGGTKLEIK |
| 101 | DIQMTQTTSSLSASLGDRVTINCRASQDISNFLNWYQQKPDGTLKLLIYYTSGL |
| | HSGVPSRFSGSGSGTDYSLTISNLEEEDIATYFCQQDNNHPYTFGSGTKLEIK |
| 102 | ENVLTQSPAIMSASLGEKVTMSCRASSSVNYMYWYQQKSDASPKLWIYYTSNL |
| | APGVPARFSGSGSGNSYSLTISSMEGEDAATYYCQQFTSSPWTFGGGTKLEIK |
| 103 | DIQMTQSPASLSASVGETVTITCRASEDIDSYLAWYQQKQGKSPQLLVYGATLL |
| | ADGVPSRFSGSGSGTQYSLKINSLQSEDVARYYCQHYYSIPYTFGSGTKLEIK |
| 104 | QIVLTQSPAIMSASPGEKVTMTCSASSSVSSRYLHWYQQKSGASPKLWIYGTSN |
| | LASGVPARFSGSGSGTSYSLTISSVEAEDAATYYCQQYHGDPLTFGAGTKLELK |
| 105 | QIVLTQSPAIMSASPGEKVTMTCSASSSVSSRYLHWYQQKSGASPKLWIYGTSN |
| | LASGVPARFSGSGSGTSYSLTISSVEAEDAASYYCQQYHGDPLTFGAGTKLELK |
| 106 | DILLTQSPAILSVSPGERVSLSCRASQTIGTSIHWYQQRTNGSIRLLIKYASES |
| | ISGIPSRFSGSGFGTDFALSINSVESEDIADYFCQQTNSWPLTFGAGTKLELK |
| 107 | QIVLTQSPAIMSASPGEKVTITCSASSRVSYMHWFQQKPGTSPKLWIYSTSNLA |
| | SGVPARFSGSGSGTSYSLTISRMEAEDAATYYCQQRSSYPLTFGAGTKLELK |

In some embodiments, the anti-CD122 antibody comprises a VH region that has HCDR sequences from a CDR series incorporated into a heavy chain framework sequence. In some embodiments, the anti-CD122 antibody comprises a VH region comprising HCDR sequences from series E7 incorporated into framework sequence F1. In some embodiments, the anti-CD122 antibody comprises a VH region comprising HCDR sequences from series E7 incorporated into framework sequence F2. In some embodiments, the anti-CD122 antibody comprises a VH region comprising HCDR sequences from series E7 incorporated into framework sequence F3. In some embodiments, the anti-CD122 antibody comprises a VH region comprising HCDR sequences from series E7 incorporated into framework sequence F4. In some embodiments, the anti-CD122 antibody comprises a VH region comprising HCDR sequences from series E7 incorporated into framework sequence F5. In some embodiments, the anti-CD122 antibody comprises a VH region comprising HCDR sequences from series E7 incorporated into framework sequence F6. In some embodiments, the anti-CD122 antibody comprises a VH region comprising HCDR sequences from series E7 incorporated into framework sequence F7.

In some embodiments, the anti-CD122 antibody comprises a VL region that has LCDR sequences from a CDR series incorporated into a light chain framework sequence. In some embodiments, the anti-CD122 antibody comprises a VL region comprising LCDR sequences from series E7 incorporated into framework sequence F8. In some embodiments, the anti-CD122 antibody comprises a VL region comprising LCDR sequences from series E7 incorporated into framework sequence F9. In some embodiments, the anti-CD122 antibody comprises a VL region comprising LCDR sequences from series E7 incorporated into framework sequence F10. In some embodiments, the anti-CD122 antibody comprises a VL region comprising LCDR sequences from series E7 incorporated into framework sequence F11. In some embodiments, the anti-CD122 antibody comprises a VL region comprising LCDR sequences from series E7 incorporated into framework sequence F12. In some embodiments, the anti-CD122 antibody comprises a VL region comprising LCDR sequences from series E7 incorporated into framework sequence F13. In some embodiments, the anti-CD122 antibody comprises a VL region comprising LCDR sequences from series E7 incorporated into framework sequence F14.

In some embodiments, the anti-CD122 antibody comprises a VH region comprising SEQ ID NO: 82 and a VL region comprising SEQ ID NO: 95. In some embodiments, the anti-CD122 antibody comprises a VH region comprising SEQ ID NO: 83 and a VL region comprising SEQ ID NO: 96. In some embodiments, the anti-CD122 antibody comprises a VH region comprising SEQ ID NO: 84 and a VL region comprising SEQ ID NO: 97. In some embodiments, the anti-CD122 antibody comprises a VH region comprising SEQ ID NO: 85 and a VL region comprising SEQ ID NO: 98. In some embodiments, the anti-CD122 antibody comprises a VH region comprising SEQ ID NO: 86 and a VL region comprising SEQ ID NO: 99. In some embodiments, the anti-CD122 antibody comprises a VH region comprising SEQ ID NO: 87 and a VL region comprising SEQ ID NO: 100. In some embodiments, the anti-CD122 antibody comprises a VH region comprising SEQ ID NO: 88 and a VL region comprising SEQ ID NO: 101. In some embodiments, the anti-CD122 antibody comprises a VH region comprising SEQ ID NO: 89 and a VL region comprising SEQ ID NO: 102. In some embodiments, the anti-CD122 antibody comprises a VH region comprising SEQ ID NO: 90 and a VL region comprising SEQ ID NO: 103. In some embodiments, the anti-CD122 antibody comprises a VH region comprising SEQ ID NO: 91 and a VL region comprising SEQ ID NO: 104. In some embodiments, the anti-CD122 antibody comprises a VH region comprising SEQ ID NO: 92 and a VL region comprising SEQ ID NO: 105. In some embodiments, the anti-CD122 antibody comprises a VH region comprising SEQ ID NO: 93 and a VL region comprising SEQ ID NO: 106. In some embodiments, the anti-CD122 antibody comprises a VH region comprising SEQ ID NO: 94 and a VL region comprising SEQ ID NO: 107.

In some embodiments, the anti-CD122 antibody comprises the VH domain encoded by a nucleic acid comprising at least 80%, sequence identity to a sequence selected from SEQ ID NOs: 108-120. In some embodiments, the anti-CD122 antibody comprises the VH domain encoded by a nucleic acid comprising at least 85% sequence identity to a sequence selected from SEQ ID NOs: 108-120. In some embodiments, the anti-CD122 antibody comprises the VH domain encoded by a nucleic acid comprising at least 90% sequence identity to a sequence selected from SEQ ID NOs: 108-120. In some embodiments, the anti-CD122 antibody comprises the VH domain encoded by a nucleic acid comprising at least 95% sequence identity to a sequence selected from SEQ ID NOs: 108-120. In some embodiments, the anti-CD122 antibody comprises the VL domain encoded by a nucleic acid comprising at least 80% sequence identity to a sequence selected from SEQ ID NOs: 121-133. In some embodiments, the anti-CD122 antibody comprises the VL domain encoded by a nucleic acid comprising at least 85% sequence identity to a sequence selected from SEQ ID NOs: 121-133. In some embodiments, the anti-CD122 antibody comprises the VL domain encoded by a nucleic acid comprising at least 90% sequence identity to a sequence selected from SEQ ID NOs: 121-133. In some embodiments, the anti-CD122 antibody comprises the VL domain encoded by a nucleic acid comprising at least 95% sequence identity to a sequence selected from SEQ ID NOs: 121-133.

Nucleic acid sequences encoding VH and VL domains are listed in Table 8. In some embodiments, the anti-CD122 antibody comprises a VH domain that is encoded by a nucleic acid comprising SEQ ID NO: 108. In some embodiments, the anti-CD122 antibody comprises a VH domain that is encoded by a nucleic acid comprising SEQ ID NO: 109. In some embodiments, the anti-CD122 antibody comprises a VH domain that is encoded by a nucleic acid comprising SEQ ID NO: 110. In some embodiments, the anti-CD122 antibody comprises a VH domain that is encoded by a nucleic acid comprising SEQ ID NO: 111. In some embodiments, the anti-CD122 antibody comprises a VH domain that is encoded by a nucleic acid comprising SEQ ID NO: 112. In some embodiments, the anti-CD122 antibody comprises a VH domain that is encoded by a nucleic acid comprising SEQ ID NO: 113. In some embodiments, the anti-CD122 antibody comprises a VH domain that is encoded by a nucleic acid comprising SEQ ID NO: 114. In some embodiments, the anti-CD122 antibody comprises a VH domain that is encoded by a nucleic acid comprising SEQ ID NO: 115. In some embodiments, the anti-CD122 antibody comprises a VH domain that is encoded by a nucleic acid comprising SEQ ID NO: 116. In some embodiments, the anti-CD122 antibody comprises a VH domain that is encoded by a nucleic acid comprising SEQ ID NO: 117. In some embodiments, the anti-CD122 antibody comprises a VH domain that is encoded by a nucleic acid comprising SEQ ID NO: 118. In some embodiments, the anti-CD122 antibody comprises a VH domain that is encoded by a nucleic acid comprising SEQ ID NO: 119. In some embodiments, the anti-CD122 antibody comprises a VH domain that is encoded by a nucleic acid comprising SEQ ID NO: 120. In some embodiments, the anti-CD122 antibody comprises a VL domain that is encoded by a nucleic acid comprising SEQ ID NO: 121. In some embodiments, the anti-CD122 antibody comprises a VL domain that is encoded by a nucleic acid comprising SEQ ID NO: 122. In some embodiments, the anti-CD122 antibody comprises a VL domain that is encoded by a nucleic acid comprising SEQ ID NO: 123. In some embodiments, the anti-CD122 antibody comprises a VL domain that is encoded by a nucleic acid comprising SEQ ID NO: 124. In some embodiments, the anti-CD122 antibody comprises a VL domain that is encoded by a nucleic acid comprising SEQ ID NO: 125. In some embodiments, the anti-CD122 antibody comprises a VL domain that is encoded by a nucleic acid comprising SEQ ID NO: 126. In some embodiments, the anti-CD122 antibody comprises a VL domain that is encoded by a nucleic acid comprising SEQ ID NO: 127. In some embodiments, the anti-CD122 antibody comprises a VL domain that is encoded by a nucleic acid comprising SEQ ID NO: 128. In some embodiments, the anti-CD122 antibody comprises a VL domain that is encoded by a nucleic acid comprising SEQ ID NO: 129. In some embodiments, the anti-CD122 antibody comprises a VL domain that is encoded by a nucleic acid comprising SEQ ID NO: 130. In some embodiments, the anti-CD122 antibody comprises a VL domain that is encoded by a nucleic acid comprising SEQ ID NO: 131. In some embodiments, the anti-CD122 antibody comprises a VL domain that is encoded by a nucleic acid comprising SEQ ID NO: 132. In some embodiments, the anti-CD122 antibody comprises a VL domain that is encoded by a nucleic acid comprising SEQ ID NO 133.

TABLE 8
Nucleic acid sequences encoding VH and VL domains

| SEQ ID | |
|---|---|
| NO: | Nucleic acid sequences encoding VH and VL domains |
| 108 | CAGGTGCAGCTGAAGGAGTCAGGACCTGGCCTGGTGGCGCCCTCACAGAGCCTG |
| | TCCATCACTTGCACCGTCTCTGGGTTTTCATTAACCAGCTATGGTATAGACTGG |
| | GTTCGCCAGCCTCCAGGAAAGGGTCTGGAGTGGCTGGGAATAATGTGGGGTGGT |
| | GGAAGCACAAATTATAATTCAGCTCTCATGTCCAGACTGAGCATCAGCAAAGAC |
| | ATCTCCAAGAGCCAAGTTTTCTTAAAAGTGAACAGTCTGCAAACTGATGACACA |
| | GCCATGTACTACTGTGCCAGACGGACCTACTCTGATTCCTATTACTATGAAATG |
| | GACTATTGGGGTCAAGGAACCTCAGTCACCGTCTCCTCA |
| 109 | CAGGTCCAACTGCAGCAGCCTGGGACTGAGCTTGTGAAGCCTGGGGCTTCAGTG |
| | AAGATGTCCTGCAAGGCTTCTGGCTACACCTTCACCAGCTACTGGATAACCTGG |
| | GTGAAGCAGAGGCCTGGACAAGGCCTTGAGTGGATTGGAGATATTTATCCTGGT |
| | CGTGGTAGTACTAACTACAATGAGAAGTTCAAGAGCAAGGCCACACTGACTGTA |
| | GACACATCCTCCAGCACAGCCTACATGCAGCTCAGCAGCCTGACATCTGAGGAC |
| | TCTGCGGTCTATTACTGTGCAAGAGAACTGGGAGGGTTTGCTTACTGGGGCCAA |
| | GGGACTCTGGTCACTGTCTCTGCA |
| 110 | CAGGTTCAGCTGCAGCAGTCTGGAGCTGAGCTGATGAAGCCTGGGGCCTCAGTG |
| | AAGATTTCCTGCAAGGCTACTGGCTCCACATTCAATAGGTACTGGATAGAGTGG |
| | GTAAAGCAGAGGCCTGGACATGGCCTTGAGTGGATTGGAGAGATTTTACCTGGA |
| | AGTGGTAATACTAATTACAATGAGAAGTTCAAGGGCAAGGCCACATTCACTGCA |
| | GATACATCCTCCAACACAGCCTACATGCAACTCAGCAGCCTGACATCTGAGGAC |
| | TCTGCCGTCTATTACTGTGCAAGATTGGATTACTACGGTAGTAGGTACTACTTT |
| | GACTACTGGGGCCAAGGCACCACTCTCACAGTCTCCTCA |
| 111 | GAGGTCCAGCTGCAACAGTCTGGACCTGAACTGGTGAAGCCTGGGGCTTCAGTG |
| | AAGATGTCCTGCAAGGCTTCTGGATTCACATTCACTGACTACAACATACACTGG |
| | GTGAAGCAGAGCCATGGAAAGAGCCTTGAGTGGATTGGATATATTAACCCTAAC |
| | AATGGTCGTTCTAGTTACAACCTGTATTTCAAGGACAAGGCCACATTGACTGTA |
| | AACAAGTCGTCCAGCACAGCCTACATGGAGCTCCGCAGCCTGACATCGGAAGAT |
| | TCTGCAGTCTATTACTGTGCAAGAGAAGACTGGGAGGGTTTTTATGCTATGGAC |
| | TACTGGGGTCAAGGAACCTCAGTCACCGTCTCCTCA |
| 112 | GAGGTCCAGCTGCAACAGTCTGGACCTGAGCTGGTGAAACCTGGAGCTTCAATG |
| | AAGATATCCTGCAAGGCTTCTGGTTACTCATTCACTGCCTACACCATGAACTGG |
| | GTGAGGCAGAGCCATGGAAAGAACCTTGAGTGGATTGGACTTATTAATCCTTAC |
| | AATGGTTATGCTAACTACAACCAGAAGTTCAAGGGCAAGGCCACATTAACTGTA |
| | GACAAGTCATCCAGTACAGCCTACATGGACCTCCTCAGTCTGACATCTGAGGAC |
| | TCTGCAGTCTATTACTGTGCAAGAGTGGGATACTACTTTGACTACTGGGGCCAA |
| | GGCACCACTCTCACAGTCTCCTCA |
| 113 | GAGGTTCAGCTGCAGCAGTCTGGGGCTGAGCTTGTGAGGCCAGGGGCCTCAGTC |
| | AAGTTGTCCTGCACATCTTCTGGCTTTAACATTAAAGACGACTATATGCACTGG |
| | GTGAAGCAGAGGCCTGAACAGGGCCTGGAGTGGATTGGATGGATTGATCCTGAG |
| | AATGGTGATACTGAATATGCCTCGAAGTTCCAGGGCAAGGCCACTATAACAGCA |
| | GACACATCCTCCAACACAGCCTACCTGCAGCTCAACAGCCTGACATCTGAGGAC |
| | ACTGCCGTCTATTACTGTACAGGCTACTTTGACTACTGGGGCCAAGGCACCACT |
| | CTCACAGTCTCCTCA |
| 114 | CAGGTCCAACTGCAGCAGCCTGGGGCTGAGCTTGTGAAGCCTGGGGCTTCAGTG |
| | AAGATGTCCTGCAAGGCTTCTGGCTACACCTTCACCAGCCACTGGATAACCTGG |
| | GTGAAGCAGAGGCCTGGACAAGGCCTTGAGTGGATTGGAGATATTTATCCTGGT |
| | AGTGGTAATACTAACTACAATGAGAAGTTCAAGAGCAAGGCCACACTGACTGTA |
| | GACACATCCTCCAGCACAGCCTACATGCAACTCAGCAGCCTGACATCTGAGGAC |
| | TCTGCGGACTATTATTGTGCAAGAGAGAGGGGAGGCTTTGACTACTGGGGCCAA |
| | GGCACCACTCTCACAGTCTCCTCA |
| 115 | GACGTAATACTGGTGGAGTCTGGGGGAGACTTAGTGAAGCCTGGAGGGTCCCTG |
| | AAACTCTCCTGTGCAGCCTCTGGATTCACTTTCAGTACCTTTGCCATGTCTTGG |
| | GTTCGCCAGACTCCGGAGAAGAGGCTGGAGTGGGTCGCAAGCATTACTGGTGAT |
| | GGTGGTACTTATACCTACTATTCAGACAGTGTGAAGGGTCGATTCACCATCTCC |
| | AGAGACAATGCCAGGAACACCCTGTACCTGCAAATGAGCAGTCTGAGGTCTGAG |
| | GACACGGCCTTCTATTACTGTGCAAGACACTCCGTTAGTAGCTGGTTTGCTTAC |
| | TGGGGCCAAGGGACTCTGGTCACTGTCTCTGCA |
| 116 | CAGGTTCAGCTGCTACAGTCTGACGCTGAGTTGGTGAAACCTGGAGCTTCAGTG |
| | AAGATCTCCTGCAAGGTTTCTGGCTTCACCTTCACTGACCATACTCTTCACTGG |
| | ATGAAGCAGAGGCCTGAACAGGGCCTGGAATGGATTGGATATATTTATCCTAGA |
| | GATGGTTATACTAAGTACAATGAGAAATTCAAGGGCAAGGCCACATTGACTGCA |
| | GACAAATCCTCCAGCACAGCCTACATGCAGCTCGACAGCCTGACATCTGAGGAC |
| | TCTGCAGTCTATTTCTGTGCAAGACCGACATCTTTACTACGGTTTCCTTACTGG |
| | GGCCAAGGGACTCTGGTCACTGTCTCTGCA |
| 117 | CAGGTGCAACTGAAGGAGTCAGGACCTGGCCTGGTGGCGCCCTCACAGAGCCTG |
| | TCCATCACTTGCACTGTCTCTGGGTTTTCATTAACCAGTTATGGTGTAGACTGG |
| | GTTCGCCAGCCTCCGGGAAAGGGTCTGGAGTGGCTGGGAGTAATATGGGGTGGT |
| | GGTGGAAGCACAAATTATAATTCAGCTCTCATGTCCAGACTGAGCATCAGCAAA |
| | GACAACTCCAAGAGTCAAGTTTTCTTAAAAATGAACAGTCTGCAAACTGATGAC |
| | ACAGCCATGTACTACTGTGCCAGACATAACTATGATGGTTATTACTATTCTTTG |
| | GACTACTGGGGTCAAGGAACCTCAGTCACCGTCTCCTCA |
| 118 | CAGGTGCAGTTGAAGGAGTCAGGACCTGGCCTGGTGGCGCCCTCACAGAGCCTG |
| | TCCATCACTTGCACTGTCTCTGGGTTTTCATTAACCAGTTATGGTGTAGACTGG |
| | GTTCGCCAGCCTCCGGGAAAGGGTCTGGAGTGGCTGGGAGTAATATGGGGTGGT |
| | GGTGGAAGCACAAATTATAATTCAGCTCTCATGTCCAGACTGAGCATCAGCAAA |
| | GACAACTCCAAGAGTCAAGTTTTCTTAAAAATGAACAGTCTGCAAACTGATGAC |
| | ACAGCCATGTACTACTGTGCCAGACATAACTATGATAATTATTACTATACTTTG |
| | GACTACTGGGGTCAAGGAACCTCAGTCACCGTCTCCTCA |
| 119 | CAGGTGCAACTGAAGGAGTCAGGACCTGGCCTGGTGGCGCCCTCACAGAGCCTG |
| | TCCATTACCTGCACTGTCTCTGGGTTCTCATTAACCAGCTATGATATAAGCTGG |
| | ATTCGCCAGCCACCAGGAAAGGGTCTGGAGTGGCTTGGAGTAATATGGACTGGT |
| | GGAGGCACAAATTATAATTCACCTTTCATGTCCAGACTGACCATCAGCAAGGAC |
| | AACTCCAGGAGCCAAGTATTCTTAAAAATGAACAGTCTGCAAACTGATGACACA |
| | GCCATGTATTACTGTGTAAGAGATCTCTTCCCCTATGCTATGGACTACTGGGGT |
| | CAAGGAACCTCAGTCACCGTCTCCTCA |
| 120 | CAGGTCCAGCTTCAGCAGTCTGGGGCTGAACTGGCAAAACCTGGGGCCTCAGTG |
| | AAGATGTCCTGCAAGGCTTCTGGCTACACCTTTACTGCCTACTGGATACATTGG |
| | GTGAAACAGAGGCCTGGACAGGGTCTAGAATGGATTGGATACATTGATCCTAAC |
| | AGTGGTTATACTGACTACAATCAGATATTCAAGGACAAGGCCACATTGACTGCA |
| | GACAGATCCTCCAGCACAGCCTACATGCAGCTGAACAGCCTGACATCTGAGGAC |
| | TCTGCAGTCTATTACTGTGCAAGAGGACACTTTGGTTACGACGACTCCTGGGGC |
| | CAAGGCATCACTCTCACAGTCTCCTCA |
| 121 | CAAATTGTTCTCACCCAGTCTCCAGCAATCATGTCTGCCTCTCCAGGGGAGAAG |
| | GTCACCATGACCTGCAGTGCCAGCTCAAGTGTAAGTTCCAGGTACTTGCACTGG |
| | TACCAGCAGAAGTCAGGAGCCTCCCCCAAACTCTGGATTTATGGCACATCCAAC |
| | CTGGCTTCTGGAGTCCCTGCTCGCTTCAGTGGCAGTGGGTCTGGGACCTCTTAC |
| | TCTCTCACAATCAGCAGCGTGGAGGCTGAAGATGCTGCCACTTATTACTGCCAG |
| | CAATATCATAGTGACCCGCTCACGTTCGGTGCTGGGACCAAGCTGGAGCTGAAG |
| 122 | CAAATTGTTCTCACCCAGTCTCCAGCAATCATGTCTGCATCTCTAGGGGAACGG |
| | GTCACCATGACCTGCACTGCCAGCTCATATGTAAGTTCCAGTTACTTACACTGG |
| | TACCAGCAGAAGCCAGGATCCTCCCCCACACTCTGGATTTATACCACATCCAAC |
| | CTGGCTTCTGGAGTCCCAGCTCGCTTCAGTGGCAGTGGGTCTGGGACCTCTTAC |
| | TCTCTCACAATCAGCAACATGGAGGCTGAAGATGCTGCCACTTATTACTGCCAC |
| | CAGTATCATCTTTCCCCATGGACGTTCGGTGGAGGCACCAAGCTGGAAATCAAA |
| 123 | GATATCCAGATGACACAGACTACATCCTCCCTGTCTGCCTCTCTGGGAGACAGA |
| | GTCACCATCAGTTGCAGGGCAAGTCAGGACATTAGGAATTATTTAAACTGGTAT |
| | CAGCAGAAACCAGATGGAACTCTTAAACTCCTGATCTATTACACATCAAGATTA |
| | CACTCAGGAGTCCCATCAAGGTTCAGTGGCAGTGGGTCTGGAACAGATTATTCT |
| | CTCACCATTAGCAACCTGGAGCAAGAAGATGTTGCCACTTACTTTTGCCAACAG |
| | GGTGATCCGCTTCCTCCGACGTTCGGTGGAGGCACCAAGGTGGAAATCAAA |
| 124 | GACATTGTGATGACCCAGTCTCAAAAATTCATGTCCACATCAGTAGGAGACAGG |
| | GTCAGCATCACCTGCAAGGCCAGTCAGAATGTGGATACTGATGTATCCTGGTAT |
| | CAACAGAAACCAGGGAAATCTCCTAAAACACTGATTTATTGGGCATCAAACCGG |
| | TTCACTGGAGTCCCTGATCGCTTCACAGGCAGTGGATCTGGGACAGATTTCACT |
| | CTCACCATCACCAATGTGCAGTCTGAAGACTTGGCAGATTATTTCTGTGAGCAA |
| | TATAGCAGCTATCCGTATACGTTCGGATCGGGGACCAAGCTGGAAATAAAA |
| 125 | GAAAATGTGCTCACCCAGTCTCCAACAATCATGTCTGCATCTCTAGGGGAGAAG |
| | GTCACCATGAGCTGCAGGGCCAGCTCAAGTGTTAGTTACATGCACTGGTACCAG |
| | CAGAAGTCAGATGCCTCCCCCAAACTTTGGATTTATTACACATCCAACCTGGCT |
| | CCTGGAGTCCCAGCTCGCTTCAGTGCCAGTGGGTCTGGGAACTCTTATTCTCTC |
| | ACAATCAGCAGCATGGAGGGTGAAGATGCTGCCACTTATTACTGCCAGCAGTTT |
| | ACTAGTTCCCCATACACGTTCGGAGGGGGGACCAAGCTGGAAATAAAA |
| 126 | GACATCCAGATGAACCAGTCTCCATCCAGTCTGTCTGCATCCCTCGGAGACACA |
| | ATTTCCATCTCTTGCCGTGCCAGTCAGAACATTAATCTTTGGTTAAACTGGTAC |
| | CAGCAGAAACCAGGAAATGTTCCTAAACTATTGATCTTTAAGGCTTCCAACTTG |
| | CACCCAGGCGTCCCATCAAGGTTTAGTGGCAGTGGATCTGGAACAGATTTCACA |
| | TTAACCATCAGCAGTCTGCAGCCTGAAGACATTGCCACTTACTACTGTCTACAG |
| | GGTCAAAGTTATCCGTGGACGTTCGGTGGAGGCACCAAGCTGGAAATCAAA |
| 127 | GATATCCAGATGACACAGACTACATCTTCCCTGTCTGCCTCTCTGGGAGACAGA |
| | GTCACCATCAATTGCAGGGCAAGTCAGGACATTAGCAATTTTTTAAACTGGTAT |
| | CAGCAGAAACCAGATGGAACTCTTAAACTCCTGATCTACTACACATCAGGATTA |
| | CACTCAGGAGTCCCATCAAGGTTCAGTGGCAGTGGGTCTGGGACAGATTATTCT |
| | CTCACTATTAGCAACCTGGAGGAAGAAGATATTGCCACTTACTTTTGCCAACAG |
| | GATAATAACCATCCTTATACGTTCGGATCGGGGACCAAGCTGGAAATAAAA |
| 128 | GAAAATGTGCTCACCCAGTCTCCAGCAATCATGTCTGCATCTCTAGGGGAGAAG |
| | GTCACCATGAGCTGCAGGGCCAGCTCAAGTGTAAATTACATGTACTGGTACCAG |
| | CAGAAGTCAGATGCCTCCCCCAAACTATGGATTTATTACACATCCAACCTGGCT |
| | CCTGGAGTCCCAGCTCGCTTCAGTGGCAGTGGGTCTGGGAACTCTTATTCTCTC |
| | ACAATCAGCAGCATGGAGGGTGAAGATGCTGCCACTTATTACTGCCAGCAGTTT |
| | ACTAGTTCCCCGTGGACGTTCGGTGGAGGCACCAAGCTGGAAATCAAA |
| 129 | GACATCCAGATGACTCAGTCTCCAGCTTCCCTGTCTGCATCTGTGGGAGAAACT |
| | GTCACCATCACATGTCGAGCAAGTGAAGATATTGACAGTTATTTAGCTTGGTAT |
| | CAACAGAAACAGGGAAAATCTCCTCAGCTCCTGGTCTATGGTGCAACACTCTTA |
| | GCAGATGGTGTGCCATCAAGGTTCAGTGGCAGTGGATCAGGCACACAGTATTCT |
| | CTCAAGATCAACAGCCTGCAGTCTGAAGATGTTGCGAGATATTACTGTCAACAT |
| | TATTATAGTATTCCGTATACGTTCGGATCGGGGACCAAGCTGGAAATAAAA |
| 130 | CAAATTGTTCTCACCCAGTCTCCAGCAATCATGTCTGCCTCTCCAGGGGAGAAG |
| | GTCACCATGACCTGCAGTGCCAGCTCAAGTGTAAGTTCCAGGTACTTGCACTGG |
| | TACCAGCAGAAGTCAGGAGCCTCCCCCAAACTCTGGATTTATGGCACATCCAAC |
| | CTGGCTTCTGGAGTCCCTGCTCGCTTCAGTGGCAGTGGGTCTGGGACCTCTTAC |
| | TCTCTCACAATCAGCAGCGTGGAGGCTGAAGATGCTGCCACTTATTACTGCCAG |
| | CAGTATCATGGTGACCCGCTCACGTTCGGTGCTGGGACCAAGCTGGAGCTGAAA |
| 131 | CAAATTGTTCTCACCCAGTCTCCAGCAATCATGTCTGCCTCTCCAGGGGAGAAG |
| | GTCACCATGACCTGCAGTGCCAGCTCAAGTGTAAGTTCCAGGTACTTGCACTGG |
| | TACCAGCAGAAGTCAGGAGCCTCCCCCAAACTCTGGATTTATGGCACATCCAAC |
| | CTGGCTTCTGGAGTCCCTGCTCGCTTCAGTGGCAGTGGGTCTGGGACCTCTTAC |
| | TCTCTCACAATCAGCAGCGTGGAGGCTGAAGATGCTGCCTCTTATTACTGCCAG |
| | CAGTATCATGGTGACCCGCTCACGTTCGGTGCTGGGACCAAGCTGGAGCTGAAA |
| 132 | GACATCCTGCTGACTCAGTCTCCAGCCATCCTGTCTGTGAGTCCAGGAGAAAGA |
| | GTCAGCCTCTCCTGCAGGGCCAGTCAGACCATTGGCACAAGCATACACTGGTAT |
| | CAGCAAAGAACAAATGGTTCTATAAGGCTTCTCATAAAATATGCTTCTGAGTCT |
| | ATCTCTGGGATCCCTTCTAGGTTTAGTGGCAGTGGATTTGGGACAGATTTTGCT |
| | CTTAGCATCAACAGTGTGGAGTCTGAAGATATTGCAGATTATTTCTGTCAACAA |
| | ACTAATAGCTGGCCACTCACGTTCGGTGCTGGGACCAAGCTGGAGCTGAAA |
| 133 | CAAATTGTTCTCACCCAGTCTCCAGCAATCATGTCTGCATCTCCAGGGGAGAAG |
| | GTCACCATAACCTGCAGTGCCAGCTCAAGAGTAAGTTACATGCACTGGTTCCAA |
| | CAAAAGCCAGGCACTTCTCCCAAACTCTGGATTTATAGCACATCCAACCTGGCT |
| | TCTGGAGTCCCTGCTCGCTTCAGTGGCAGTGGATCTGGGACCTCTTATTCTTTA |
| | ACAATCAGCCGAATGGAGGCTGAAGATGCTGCCACTTATTACTGCCAGCAAAGG |
| | AGTAGTTACCCACTCACGTTCGGTGCTGGGACCAAGCTGGAGCTGAAA |

In some embodiments, an anti-CD122 antibody described herein is a full-length antibody. In other aspects, the anti-CD122 antibody is an antigen binding fragment thereof. In some cases, the anti-CD122 antibody is a humanized antibody or an antigen binding fragment thereof, a chimeric antibody or an antigen binding fragment thereof, a monoclonal antibody or an antigen binding fragment thereof. In some cases, the anti-CD122 antibody is monovalent Fab′, F(ab)′3 fragments, single-chain variable fragment (scFv), (scFv)2, minibody, nanobody, disulfide stabilized Fv protein (“dsFv”), single-domain antibody (sdAb), Ig NAR, camelid antibody or an antigen binding fragment thereof, or a chemically modified derivative thereof.

In some cases, an anti-CD122 antibody comprises one or more mutations in a framework region, e.g., in the CH1 domain, CH2 domain, CH3 domain, hinge region, or a combination thereof. In some instances, the one or more mutations are to stabilize the antibody. In some instances, the one or more mutations are to increase half-life. In some instances, the one or more mutations are to modulate Fc receptor interactions. In some instances, the one or more mutations are to reduce or eliminate Fc effector functions such as FcyR, antibody-dependent cell-mediated cytotoxicity (ADCC), or complement-dependent cytotoxicity (CDC). In additional instances, the one or more mutations are to modulate glycosylation.

In some embodiments, the one or more mutations are located in the Fc region.

In some embodiments, the human IgG constant region is modified to alter antibody-dependent cellular cytotoxicity (ADCC) and/or complement-dependent cytotoxicity (CDC), e.g., with an amino acid modification described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Urban et al., 2021 Front Immunol. November 25; 12:724361; Zhou et al., 2020 MAbs. January-December; 12(1):1814583; Reviewed in Kaneko and Niwa, 2011 Biodrugs, 25(1): 1-11.

In some embodiments, an anti-CD122 antibody described herein is a full-length antibody, comprising a heavy chain (HC) and a light chain (LC). In some embodiments, the HC comprises about 80%, 85%, 90%, 95%, 96% 97%, 98%, 99%, or 100% sequence identity to a sequence selected from SEQ ID NOs: 134-141. In some embodiments, the anti-CD122 antibody comprises the heavy chain comprising at least 80% sequence identity to a sequence selected from SEQ ID NOs: 134-141. In some embodiments, the anti-CD122 antibody comprises the heavy chain comprising at least 85% sequence identity to a sequence selected from SEQ ID NOs: 134-141. In some embodiments, the anti-CD122 antibody comprises the heavy chain comprising at least 90% sequence identity to a sequence selected from SEQ ID NOs: 134-141. In some embodiments, the anti-CD122 antibody comprises the heavy chain comprising at least 90% sequence identity to SEQ ID NO: 134. In some embodiments, the anti-CD122 antibody comprises the heavy chain comprising at least 90% sequence identity to SEQ ID NO: 135. In some embodiments, the anti-CD122 antibody comprises the heavy chain comprising at least 90% sequence identity to SEQ ID NO: 136. In some embodiments, the anti-CD122 antibody comprises the heavy chain comprising at least 90% sequence identity to SEQ ID NO: 137. In some embodiments, the anti-CD122 antibody comprises the heavy chain comprising at least 90% sequence identity to SEQ ID NO: 138. In some embodiments, the anti-CD122 antibody comprises the heavy chain comprising at least 90% sequence identity to SEQ ID NO: 139. In some embodiments, the anti-CD122 antibody comprises the heavy chain comprising at least 90% sequence identity to SEQ ID NO: 140. In some embodiments, the anti-CD122 antibody comprises the heavy chain comprising at least 90% sequence identity to SEQ ID NO: 141. In some embodiments, the anti-CD122 antibody comprises the heavy chain comprising at least 95% sequence identity to a sequence selected from SEQ ID NOs: 134-141. In some embodiments, the anti-CD122 antibody comprises the heavy chain comprising at least 96% sequence identity to a sequence selected from SEQ ID NOs: 134-141. In some embodiments, the anti-CD122 antibody comprises the heavy chain comprising at least 97% sequence identity to a sequence selected from SEQ ID NOs: 134-141. In some embodiments, the anti-CD122 antibody comprises the heavy chain comprising at least 98% sequence identity to a sequence selected from SEQ ID NOs: 134-141. In some embodiments, the anti-CD122 antibody comprises the heavy chain comprising at least 99% sequence identity to a sequence selected from SEQ ID NOs: 134-141. In some embodiments, the anti-CD122 antibody comprises the heavy chain comprising 100% sequence identity to a sequence selected from SEQ ID NOs: 134-141. In some embodiments, the VH domain of the anti-CD122 antibody comprises at least 80%, at least 85%, at least 90%, at least 95%, at least 96%, at least 97%, at least 98%, or at least 99% sequence identity to a VH domain found in a sequence selected from SEQ ID NOs: 134-141. In some embodiments, the VH domain of the anti-CD122 antibody comprises at least 90% sequence identity to a VH domain found SEQ ID NO: 134. In some embodiments, the VH domain of the anti-CD122 antibody comprises at least 90% sequence identity to a VH domain found SEQ ID NO: 135. In some embodiments, the VH domain of the anti-CD122 antibody comprises at least 90% sequence identity to a VH domain found SEQ ID NO: 136. In some embodiments, the VH domain of the anti-CD122 antibody comprises at least 90% sequence identity to a VH domain found SEQ ID NO: 137. In some embodiments, the VH domain of the anti-CD122 antibody comprises at least 90% sequence identity to a VH domain found SEQ ID NO: 138. In some embodiments, the VH domain of the anti-CD122 antibody comprises at least 90% sequence identity to a VH domain found SEQ ID NO: 139. In some embodiments, the VH domain of the anti-CD122 antibody comprises at least 90% sequence identity to a VH domain found SEQ ID NO: 140. In some embodiments, the VH domain of the anti-CD122 antibody comprises at least 90% sequence identity to a VH domain found SEQ ID NO: 141. In some embodiments, the VH domain of the anti-CD122 antibody comprises 100% sequence identity to a VH domain found SEQ ID NO: 134. In some embodiments, the VH domain of the anti-CD122 antibody comprises 100% sequence identity to a VH domain found SEQ ID NO: 135. In some embodiments, the VH domain of the anti-CD122 antibody comprises at least 100% sequence identity to a VH domain found SEQ ID NO: 136. In some embodiments, the VH domain of the anti-CD122 antibody comprises 100% sequence identity to a VH domain found SEQ ID NO: 137. In some embodiments, the VH domain of the anti-CD122 antibody comprises 100% sequence identity to a VH domain found SEQ ID NO: 138. In some embodiments, the VH domain of the anti-CD122 antibody comprises 100% sequence identity to a VH domain found SEQ ID NO: 139. In some embodiments, the VH domain of the anti-CD122 antibody comprises 100% sequence identity to a VH domain found SEQ ID NO: 140. In some embodiments, the VH domain of the anti-CD122 antibody comprises 100% sequence identity to a VH domain found SEQ ID NO: 141. In some embodiments, the heavy chain comprises one or more conservative amino acid substitutions from a sequence described herein. In some embodiments, the VH domain comprises one or more conservative amino acid substitutions from a sequence described herein.

In some embodiments, an anti-CD122 antibody described herein is a full-length antibody, comprising a heavy chain (HC) and a light chain (LC). In some embodiments, the LC comprises about 80%, 85%, 90%, 95%, 96% 97%, 98%, 99%, or 100% sequence identity to a sequence selected from SEQ ID NOs: 142-145. In some embodiments, the anti-CD122 antibody comprises the light chain comprising at least 80%, at least 85%, at least 90%, or at least 95% sequence identity to a sequence selected from SEQ ID NOs: 142-145. In some embodiments, the anti-CD122 antibody comprises the light chain comprising at least 80% sequence identity to a sequence selected from SEQ ID NOs: 142-145. In some embodiments, the anti-CD122 antibody comprises the light chain comprising at least 85% sequence identity to a sequence selected from SEQ ID NOs: 142-145. In some embodiments, the anti-CD122 antibody comprises the light chain comprising at least 90% sequence identity to a sequence selected from SEQ ID NOs: 142-145. In some embodiments, the anti-CD122 antibody comprises the light chain comprising at least 90% sequence identity to SEQ ID NO: 142. In some embodiments, the anti-CD122 antibody comprises the light chain comprising at least 90% sequence identity to SEQ ID NO: 143. In some embodiments, the anti-CD122 antibody comprises the light chain comprising at least 90% sequence identity to SEQ ID NO: 144. In some embodiments, the anti-CD122 antibody comprises the light chain comprising at least 90% sequence identity to SEQ ID NO: 145. In some embodiments, the anti-CD122 antibody comprises the light chain comprising at least 95% sequence identity to a sequence selected from SEQ ID NOs: 142-145. In some embodiments, the anti-CD122 antibody comprises the light chain comprising at least 96% sequence identity to a sequence selected from SEQ ID NOs: 142-145. In some embodiments, the anti-CD122 antibody comprises the light chain comprising at least 97% sequence identity to a sequence selected from SEQ ID NOs: 142-145. In some embodiments, the anti-CD122 antibody comprises the light chain comprising at least 98% sequence identity to a sequence selected from SEQ ID NOs: 142-145. In some embodiments, the anti-CD122 antibody comprises the light chain comprising at least 99% sequence identity to a sequence selected from SEQ ID NOs: 142-145. In some embodiments, the VL domain of the anti-CD122 antibody comprises at least 80%, at least 85%, at least 90%, at least 95%, at least 96%, at least 97%, at least 98%, or at least 99% sequence identity to a VL domain found in a sequence selected from SEQ ID NOs: 142-145. In some embodiments, the VL domain of the anti-CD122 antibody comprises at least 90% sequence identity to a VL domain found SEQ ID NO: 142. In some embodiments, the VL domain of the anti-CD122 antibody comprises at least 90% sequence identity to a VL domain found SEQ ID NO: 143. In some embodiments, the VL domain of the anti-CD122 antibody comprises at least 90% sequence identity to a VL domain found SEQ ID NO: 144. In some embodiments, the VL domain of the anti-CD122 antibody comprises at least 90% sequence identity to a VL domain found SEQ ID NO: 145. In some embodiments, the VL domain of the anti-CD122 antibody comprises 100% sequence identity to a VL domain found SEQ ID NO: 142. In some embodiments, the VL domain of the anti-CD122 antibody comprises 100% sequence identity to a VL domain found SEQ ID NO: 143. In some embodiments, the VL domain of the anti-CD122 antibody comprises 100% sequence identity to a VL domain found SEQ ID NO: 144. In some embodiments, the VL domain of the anti-CD122 antibody comprises 100% sequence identity to a VL domain found SEQ ID NO: 145. In some embodiments, the light chain comprises one or more conservative amino acid substitutions from a sequence described herein. In some embodiments, the VL domain comprises one or more conservative amino acid substitutions from a sequence described herein.

In some cases for an anti-CD122 antibody described herein, the heavy chain (HC) comprises a sequence selected from Table 9. In some cases for an anti-CD122 antibody described herein, the light chain (LC) comprises a sequence selected from Table 10. In some embodiments, a HC with a VH domain with a mouse HC variable framework sequence and HCDR sequences is within an IgG1 backbone and is listed as SEQ ID NO: 134. In some embodiments, a HC with a VH domain with a mouse HC variable framework sequence and HCDR sequences is within an IgG4 backbone and is listed as SEQ ID NO: 138. In some embodiments, a LC with a VL domain with a mouse light chain variable framework sequence and LCDR sequences is within an kappa light chain backbone and is listed as SEQ ID NO: 142.

TABLE 9
anti-CD122 antibody heavy chain sequences

| SEQ ID | |
|---|---|
| NO: | Heavy Chain sequence |
| 134 | (X)_{n = 14-40} |
| | QVQLQQPGAELVKPGASVKMSCKASGYTFTSHWITWVKQRPGQGLEWIGDIY |
| | PGSGNTNYNEKFKSKATLTVDTSSSTAYMQLSSLTSEDSADYYCARERGGFDY |
| | WGQGTTLTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKK |
| | VEPKSCDKTHTCPPCPAPELLGGPSVFLFPPKPKDTLMISRTPEVTCVVVDVSHE |
| | DPEVKFNWYVDGVEVHNAKTKPREEQYNSTYRVVSVLTVLHQDWLNGKEYK |
| | CKVSNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCSVM |
| | HEALHNHYTQKSLSLSPG* |
| 135 | (X)_{n = 14-40} |
| | QVQLVQSGAEVKKPGASVKVSCKASGYTFTSHWISWVRQAPGQGLEWMGDIY |
| | PGSGNTNYNEKLQGRVTMTVDTSTSTAYMELRSLRSDDTAVYYCARERGGFD |
| | YWGQGTLVTVSSASTKGPSVFPLAPSSKSTSGGTAALGCLVKDYFPEPVTVSW |
| | NSGALTSGVHTFPAVLQSSGLYSLSSVVTVPSSSLGTQTYICNVNHKPSNTKVD |
| | KKVEPKSCDKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQDWLNGKE |
| | YKCKVSNKALPAPIEKTISKAKGQPREPQVYTLPPSREEMTKNQVSLTCLVKGF |
| | YPSDIAVEWESNGQPENNYKTTPPVLDSDGSFFLYSKLTVDKSRWQQGNVFSC |
| | SVMHEALHNHYTQKSLSLSPG* |
| 136 | (X)_{n = 14-40} |
| | QVQLVQSGAEVKKPGASVKVSCKASGYTFTSHWITWVRQAPGQGLEWMGDIY |
| | PGSGNTNYNEKFQGRVTLTVDTSISTAYMELSRLRSDDTVVYYCARERGGFDY |
| | WGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKK |
| | VEPKSCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKEYKC |
| | KVSNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCSVM |
| | HEALHNHYTQKSLSLSPG* |
| 137 | (X)_{n = 14-40} |
| | QVQLVQSGAEVKKPGASVKVSCKASGYTFTSHWITWVRQAPGQRLEWMGDIY |
| | PGSGNTNYNEKFQGRVTITVDTSASTAYMELSSLRSEDTAVYYCARERGGFDY |
| | WGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKK |
| | VEPKSCDKTHTCPPCPAPELLGGPSVFLFPPKPKDTLMISRTPEVTCVVVDVSHE |
| | DPEVKFNWYVDGVEVHNAKTKPREEQYNSTYRVVSVLTVLHQDWLNGKEYKC |
| | KVSNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCSVM |
| | HEALHNHYTQKSLSLSPG* |
| 138 | (X)_{n = 14-40} |
| | QVQLQQPGAELVKPGASVKMSCKASGYTFTSHWITWVKQRPGQGLEWIGDIY |
| | PGSGNTNYNEKFKSKATLTVDTSSSTAYMQLSSLTSEDSADYYCARERGGFDY |
| | WGQGTTLTVSSASTKGPSVFPLAPCSRSTSESTAALGCLVKDYFPEPVTVSWNS |
| | GALTSGVHTFPAVLQSSGLYSLSSVVTVPSSSLGTKTYTCNVDHKPSNTKVDKR |
| | VESKYGPPCPPCPAPEFLGGPSVFLFPPKPKDTLMISRTPEVTCVVVDVSQEDPE |
| | VQFNWYVDGVEVHNAKTKPREEQFNSTYRVVSVLTVLHQDWLNGKEYKCKV |
| | SNKGLPSSIEKTISKAKGQPREPQVYTLPPSQEEMTKNQVSLTCLVKGFYPSDIA |
| | VEWESNGQPENNYKTTPPVLDSDGSFFLYSRLTVDKSRWQEGNVFSCSVMHEA |
| | LHNHYTQKSLSLSLG* |
| 139 | (X)_{n = 14-40} |
| | QVQLVQSGAEVKKPGASVKVSCKASGYTFTSHWISWVRQAPGQGLEWMGDIY |
| | PGSGNTNYNEKLQGRVTMTVDTSTSTAYMELRSLRSDDTAVYYCARERGGFD |
| | YWGQGTLVTVSSASTKGPSVFPLAPCSRSTSESTAALGCLVKDYFPEPVTVSWN |
| | SGALTSGVHTFPAVLQSSGLYSLSSVVTVPSSSLGTKTYTCNVDHKPSNTKVDK |
| | RVESKYGPPCPPCPAPEFLGGPSVFLFPPKPKDTLMISRTPEVTCVVVDVSQEDP |
| | EVQFNWYVDGVEVHNAKTKPREEQFNSTYRVVSVLTVLHQDWLNGKEYKCKV |
| | SNKGLPSSIEKTISKAKGQPREPQVYTLPPSQEEMTKNQVSLTCLVKGFYPSDI |
| | AVEWESNGQPENNYKTTPPVLDSDGSFFLYSRLTVDKSRWQEGNVFSCSVMHE |
| | ALHNHYTQKSLSLSLG* |
| 140 | (X)_{n = 14-40} |
| | QVQLVQSGAEVKKPGASVKVSCKASGYTFTSHWITWVRQAPGQGLEWMGDIY |
| | PGSGNTNYNEKFQGRVTLTVDTSISTAYMELSRLRSDDTVVYYCARERGGFDY |
| | WGQGTLVTVSSASTKGPSVFPLAPCSRSTSESTAALGCLVKDYFPEPVTVSWNS |
| | GALTSGVHTFPAVLQSSGLYSLSSVVTVPSSSLGTKTYTCNVDHKPSNTKVDKR |
| | VESKYGPPCPPCPAPEFLGGPSVFLFPPKPKDTLMISRTPEVTCVVVDVSQEDPE |
| | VQFNWYVDGVEVHNAKTKPREEQFNSTYRVVSVLTVLHQDWLNGKEYKCKVS |
| | NKGLPSSIEKTISKAKGQPREPQVYTLPPSQEEMTKNQVSLTCLVKGFYPSDIA |
| | VEWESNGQPENNYKTTPPVLDSDGSFFLYSRLTVDKSRWQEGNVFSCSVMHEA |
| | LHNHYTQKSLSLSLG* |
| 141 | (X)_{n = 14-40} |
| | QVQLVQSGAEVKKPGASVKVSCKASGYTFTSHWITWVRQAPGQRLEWMGDIY |
| | PGSGNTNYNEKFQGRVTITVDTSASTAYMELSSLRSEDTAVYYCARERGGFDY |
| | WGQGTLVTVSSASTKGPSVFPLAPCSRSTSESTAALGCLVKDYFPEPVTVSWNS |
| | GALTSGVHTFPAVLQSSGLYSLSSVVTVPSSSLGTKTYTCNVDHKPSNTKVDKR |
| | VESKYGPPCPPCPAPEFLGGPSVFLFPPKPKDTLMISRTPEVTCVVVDVSQEDP |
| | EVQFNWYVDGVEVHNAKTKPREEQFNSTYRVVSVLTVLHQDWLNGKEYKCKVS |
| | NKGLPSSIEKTISKAKGQPREPQVYTLPPSQEEMTKNQVSLTCLVKGFYPSDIA |
| | VEWESNGQPENNYKTTPPVLDSDGSFFLYSRLTVDKSRWQEGNVFSCSVMHEA |
| | LHNHYTQKSLSLSLG* |

(X)_{n = 14-40} is a leader sequence

TABLE 10
anti-CD122 antibody light chain sequences

| SEQ ID | |
|---|---|
| NO: | Light Chain sequence |
| 142 | (X)ₙ _{= 14-40} |
| | DIQMTQTTSSLSASLGDRVTINCRASQDISNFLNWYQQKPDGTLKLLIYYTSGLH |
| | SGVPSRFSGSGSGTDYSLTISNLEEEDIATYFCQQDNNHPYTFGSGTKLEIKRTV |
| | AAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC* |
| 143 | (X)_{n = 14-40} |
| | DIQMTQSPSSLSASVGDRVTITCRASQDISNFLNWYQQKPEGTLKLLIYYTSGLH |
| | SGVPSRFSGSGSGTDFTLTISSLQPEDVATYYCQQDNNHPYTFGQGTKLEIKRTV |
| | AAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC* |
| 144 | (X)_{n = 14-40} |
| | DIQMTQSPSSLSASVGDRVTITCQASQDISNFLNWYQQKPGKTLKLLIYYTSGLH |
| | TGVPSRFSGSGSGTDFTFTISSLQPEDIATYYCQQDNNHPYTFGQGTKLEIKRTV |
| | AAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC* |
| 145 | (X)_{n = 14-40} |
| | DIQMTQSPSSLSASVGDRVTITCRASQDISNFLNWYQQKPGKAPKLLIYYTSGLH |
| | SGVPSRFSGSGSGTDFTLTISSLQPEDFATYYCQQDNNHPYTFGQGTKLEIKRTV |
| | AAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC* |

(X)_{n = 14-40} is a leader sequence

In some embodiments, an anti-CD122 antibody described herein is a full-length antibody, comprising a heavy chain (HC) and a light chain (LC). In some embodiments, the full-length antibody comprises the HC comprising SEQ ID NO: 134 and the LC comprising SEQ ID NO: 142. In some embodiments, the full-length antibody comprises the HC comprising SEQ ID NO: 135 and the LC comprising SEQ ID NO: 143. In some embodiments, the full-length antibody comprises the HC comprising SEQ ID NO: 135 and the LC comprising SEQ ID NO: 144. In some embodiments, the full-length antibody comprises the HC comprising SEQ ID NO: 135 and the LC comprising SEQ ID NO: 145. In some embodiments, the full-length antibody comprises the HC comprising SEQ ID NO: 136 and the LC comprising SEQ ID NO: 143. In some embodiments, the full-length antibody comprises the HC comprising SEQ ID NO: 136 and the LC comprising SEQ ID NO: 144. In some embodiments, the full-length antibody comprises the HC comprising SEQ ID NO: 136 and the LC comprising SEQ ID NO: 145. In some embodiments, the full-length antibody comprises the HC comprising SEQ ID NO: 137 and the LC comprising SEQ ID NO: 143. In some embodiments, the full-length antibody comprises the HC comprising SEQ ID NO: 137 and the LC comprising SEQ ID NO: 144. In some embodiments, the full-length antibody comprises the HC comprising SEQ ID NO: 137 and the LC comprising SEQ ID NO: 145. In some embodiments, the full-length antibody comprises the HC comprising SEQ ID NO: 138 and the LC comprising SEQ ID NO: 142. In some embodiments, the full-length antibody comprises the HC comprising SEQ ID NO: 139 and the LC comprising SEQ ID NO: 143. In some embodiments, the full-length antibody comprises the HC comprising SEQ ID NO: 139 and the LC comprising SEQ ID NO: 144. In some embodiments, the full-length antibody comprises the HC comprising SEQ ID NO: 139 and the LC comprising SEQ ID NO: 145. In some embodiments, the full-length antibody comprises the HC comprising SEQ ID NO: 140 and the LC comprising SEQ ID NO: 143. In some embodiments, the full-length antibody comprises the HC comprising SEQ ID NO: 140 and the LC comprising SEQ ID NO: 144. In some embodiments, the full-length antibody comprises the HC comprising SEQ ID NO: 140 and the LC comprising SEQ I D NO: 145. In some embodiments, the full-length antibody comprises the HC comprising SEQ ID NO: 141 and the LC comprising SEQ ID NO: 143. In some embodiments, the full-length antibody comprises the HC comprising SEQ ID NO: 141 and the LC comprising SEQ ID NO: 144. In some embodiments, the full-length antibody comprises the HC comprising SEQ ID NO: 129 and the LC comprising SEQ ID NO: 145.

In some embodiments, the anti-CD122 antibody comprises a full length antibody chain polypeptides that are encoded by a nucleic acid comprising at least 80%, at least 85%, at least 90%, or at least 95% sequence identity to a sequence selected from SEQ ID NOs: 146-157. In some embodiments, the full length antibody chain is encoded by a heavy chain nucleic acid sequence selected from SEQ ID NOs: 146-153. In some embodiments, the full length antibody chain is encoded by a light chain nucleic acid sequence selected from SEQ ID NOs: 154-157. In some embodiments, the anti-CD122 antibody comprises the heavy chain encoded by a nucleic acid comprising at least 80% sequence identity to a sequence selected from SEQ ID NOs: 146-153. In some embodiments, the anti-CD122 antibody comprises the heavy chain encoded by a nucleic acid comprising at least 85% sequence identity to a sequence selected from SEQ ID NOs: 146-153. In some embodiments, the anti-CD122 antibody comprises the heavy chain encoded by a nucleic acid comprising at least 90% sequence identity to a sequence selected from SEQ ID NOs: 146-153. In some embodiments, the anti-CD122 antibody comprises the heavy chain encoded by a nucleic acid comprising at least 95% sequence identity to a sequence selected from SEQ ID NOs: 146-153. In some embodiments, the anti-CD122 antibody comprises the light chain encoded by a nucleic acid comprising at least 80% sequence identity to a sequence selected from SEQ ID NOs: 154-157. In some embodiments, the anti-CD122 antibody comprises the light chain encoded by a nucleic acid comprising at least 85% sequence identity to a sequence selected from SEQ ID NOs: 154-157. In some embodiments, the anti-CD122 antibody comprises the light chain encoded by a nucleic acid comprising at least 90% sequence identity to a sequence selected from SEQ ID NOs: 154-157. In some embodiments, the anti-CD122 antibody comprises the light chain encoded by a nucleic acid comprising at least 95% sequence identity to a sequence selected from SEQ ID NOs: 154-157. In some embodiments, the full length antibody chain that is encoded by a nucleic acid is selected from Table 11.

TABLE 11
Nucleic acid sequences encoding full length heavy and light chains

| | |
|---|---|
| SEQ | |
| ID | Nucleic acid sequences encoding heavy chains and light chains |
| NO: | |
| 146 | (X)_{n = 42-120} |
| | CAGGTGCAGCTCCAGCAGCCCGGAGCGGAGCTGGTTAAGCCTGGCGCAAGCGTGA |
| | AGATGAGTTGCAAGGCCAGCGGCTACACCTTCACCAGCCACTGGATCACCTGGGT |
| | GAAGCAGAGGCCGGGACAGGGCCTGGAGTGGATCGGCGACATCTACCCCGGCAGC |
| | GGCAACACCAACTACAACGAGAAGTTCAAGAGTAAGGCCACACTGACCGTGGACA |
| | CCAGTAGCAGCACCGCCTACATGCAGCTGAGCAGCCTGACCAGCGAGGACAGCGC |
| | CGACTACTACTGTGCCCGCGAGAGGGGCGGATTCGACTATTGGGGCCAAGGCACC |
| | ACCTTGACCGTCAGCTCTGCTAGCACCAAGGGCCCCAGCGTGTTCCCTCTGGCCC |
| | CCAGCAGCAAGAGCACCAGCGGCGGAACCGCCGCCCTGGGCTGCCTGGTGAAGGA |
| | CTACTTCCCCGAGCCCGTGACCGTGTCCTGGAACAGCGGCGCTCTGACCAGCGGA |
| | GTGCACACCTTCCCTGCCGTGCTGCAGAGCAGCGGCCTGTACTCCCTGAGCAGCG |
| | TGGTGACCGTGCCCAGCAGCAGCCTGGGCACCCAGACCTACATCTGCAACGTGAA |
| | CCACAAGCCCTCCAACACCAAGGTGGACAAGAAGGTGGAGCCTAAGAGCTGCGAC |
| | AAGACCCACACCTGCCCTCCCTGCCCCGCCCCCGAGCTGCTGGGCGGACCCAGCG |
| | TGTTCCTGTTCCCTCCCAAGCCCAAGGACACCCTGATGATCAGCCGCACCCCCGA |
| | GGTGACCTGCGTGGTGGTGGACGTGAGCCACGAGGACCCCGAGGTGAAGTTCAAC |
| | TGGTACGTGGACGGCGTGGAGGTGCACAACGCCAAGACCAAGCCTCGGGAGGAGC |
| | AGTACAACTCCACCTACCGCGTGGTGAGCGTGCTGACCGTGCTGCACCAGGACTG |
| | GCTGAACGGCAAGGAGTACAAGTGCAAGGTGAGCAACAAGGCCCTGCCCGCTCCC |
| | ATCGAGAAGACCATCAGCAAGGCCAAGGGCCAGCCCCGGGAGCCTCAGGTGTACA |
| | CCCTGCCCCCCAGCCGCGAAGAGATGACCAAGAACCAGGTGAGCCTGACCTGCCT |
| | GGTGAAGGGCTTCTACCCCTCCGACATCGCCGTGGAGTGGGAGAGCAACGGCCAG |
| | CCTGAGAACAACTACAAGACCACCCCTCCCGTGCTGGACAGCGACGGCAGCTTCT |
| | TCCTGTACAGCAAGCTGACCGTGGACAAGTCCCGGTGGCAGCAGGGCAACGTGTT |
| | CAGCTGCAGCGTGATGCACGAGGCCCTGCACAACCACTACACCCAGAAGAGCCTG |
| | AGCCTGAGCCCCGGATAG |
| 147 | (X)_{n = 42-120} |
| | CAGGTGCAGCTGGTGCAGAGCGGAGCAGAGGTGAAGAAGCCCGGAGCGAGCGTGA |
| | AGGTGAGCTGCAAGGCCAGCGGCTACACCTTTACCAGCCACTGGATCAGCTGGGT |
| | GAGGCAAGCCCCTGGACAGGGCCTGGAGTGGATGGGCGACATCTACCCCGGCAGC |
| | GGCAACACAAACTACAACGAGAAGCTGCAGGGCAGGGTGACCATGACCGTGGACA |
| | CCAGTACCAGCACAGCCTACATGGAGCTGAGGAGCCTCAGGAGCGACGACACCGC |
| | CGTGTACTACTGCGCCAGGGAAAGGGGCGGATTCGACTACTGGGGCCAGGGCACC |
| | CTGGTGACCGTTAGCAGCGCTAGCACCAAGGGCCCCAGCGTGTTCCCTCTGGCCC |
| | CCAGCAGCAAGAGCACCAGCGGCGGAACCGCCGCCCTGGGCTGCCTGGTGAAGGA |
| | CTACTTCCCCGAGCCCGTGACCGTGTCCTGGAACAGCGGCGCTCTGACCAGCGGA |
| | GTGCACACCTTCCCTGCCGTGCTGCAGAGCAGCGGCCTGTACTCCCTGAGCAGCG |
| | TGGTGACCGTGCCCAGCAGCAGCCTGGGCACCCAGACCTACATCTGCAACGTGAA |
| | CCACAAGCCCTCCAACACCAAGGTGGACAAGAAGGTGGAGCCTAAGAGCTGCGAC |
| | AAGACCCACACCTGCCCTCCCTGCCCCGCCCCCGAGCTGCTGGGCGGACCCAGCG |
| | TGTTCCTGTTCCCTCCCAAGCCCAAGGACACCCTGATGATCAGCCGCACCCCCGA |
| | GGTGACCTGCGTGGTGGTGGACGTGAGCCACGAGGACCCCGAGGTGAAGTTCAAC |
| | TGGTACGTGGACGGCGTGGAGGTGCACAACGCCAAGACCAAGCCTCGGGAGGAGC |
| | AGTACAACTCCACCTACCGCGTGGTGAGCGTGCTGACCGTGCTGCACCAGGACTG |
| | GCTGAACGGCAAGGAGTACAAGTGCAAGGTGAGCAACAAGGCCCTGCCCGCTCCC |
| | ATCGAGAAGACCATCAGCAAGGCCAAGGGCCAGCCCCGGGAGCCTCAGGTGTACA |
| | CCCTGCCCCCCAGCCGCGAAGAGATGACCAAGAACCAGGTGAGCCTGACCTGCCT |
| | GGTGAAGGGCTTCTACCCCTCCGACATCGCCGTGGAGTGGGAGAGCAACGGCCAG |
| | CCTGAGAACAACTACAAGACCACCCCTCCCGTGCTGGACAGCGACGGCAGCTTCT |
| | TCCTGTACAGCAAGCTGACCGTGGACAAGTCCCGGTGGCAGCAGGGCAACGTGTT |
| | CAGCTGCAGCGTGATGCACGAGGCCCTGCACAACCACTACACCCAGAAGAGCCTG |
| | AGCCTGAGCCCCGGATAG |
| 148 | (X)_{n = 42-120} |
| | CAGGTGCAGCTGGTGCAGAGCGGAGCAGAGGTGAAGAAGCCCGGAGCGAGCGTGA |
| | AGGTGAGCTGCAAGGCCAGCGGCTACACCTTTACCAGCCACTGGATCACCTGGGT |
| | GAGGCAAGCCCCTGGACAGGGCCTGGAGTGGATGGGCGACATCTACCCCGGCAGC |
| | GGCAACACAAACTACAACGAGAAGTTCCAGGGCAGGGTGACCTTGACCGTGGACA |
| | CCAGTATCAGCACAGCCTACATGGAGCTGAGTAGACTCAGGAGCGACGACACCGT |
| | CGTGTACTACTGCGCCAGGGAAAGGGGCGGATTCGACTACTGGGGCCAGGGCACC |
| | CTGGTGACCGTTAGCAGCGCTAGCACCAAGGGCCCCAGCGTGTTCCCTCTGGCCC |
| | CCAGCAGCAAGAGCACCAGCGGCGGAACCGCCGCCCTGGGCTGCCTGGTGAAGGA |
| | CTACTTCCCCGAGCCCGTGACCGTGTCCTGGAACAGCGGCGCTCTGACCAGCGGA |
| | GTGCACACCTTCCCTGCCGTGCTGCAGAGCAGCGGCCTGTACTCCCTGAGCAGCG |
| | TGGTGACCGTGCCCAGCAGCAGCCTGGGCACCCAGACCTACATCTGCAACGTGAA |
| | CCACAAGCCCTCCAACACCAAGGTGGACAAGAAGGTGGAGCCTAAGAGCTGCGAC |
| | AAGACCCACACCTGCCCTCCCTGCCCCGCCCCCGAGCTGCTGGGCGGACCCAGCG |
| | TGTTCCTGTTCCCTCCCAAGCCCAAGGACACCCTGATGATCAGCCGCACCCCCGA |
| | GGTGACCTGCGTGGTGGTGGACGTGAGCCACGAGGACCCCGAGGTGAAGTTCAAC |
| | TGGTACGTGGACGGCGTGGAGGTGCACAACGCCAAGACCAAGCCTCGGGAGGAGC |
| | AGTACAACTCCACCTACCGCGTGGTGAGCGTGCTGACCGTGCTGCACCAGGACTG |
| | GCTGAACGGCAAGGAGTACAAGTGCAAGGTGAGCAACAAGGCCCTGCCCGCTCCC |
| | ATCGAGAAGACCATCAGCAAGGCCAAGGGCCAGCCCCGGGAGCCTCAGGTGTACA |
| | CCCTGCCCCCCAGCCGCGAAGAGATGACCAAGAACCAGGTGAGCCTGACCTGCCT |
| | GGTGAAGGGCTTCTACCCCTCCGACATCGCCGTGGAGTGGGAGAGCAACGGCCAG |
| | CCTGAGAACAACTACAAGACCACCCCTCCCGTGCTGGACAGCGACGGCAGCTTCT |
| | TCCTGTACAGCAAGCTGACCGTGGACAAGTCCCGGTGGCAGCAGGGCAACGTGTT |
| | CAGCTGCAGCGTGATGCACGAGGCCCTGCACAACCACTACACCCAGAAGAGCCTG |
| | AGCCTGAGCCCCGGATAG |
| 149 | (X)_{n = 42-120} |
| | CAGGTGCAGCTGGTGCAGAGCGGAGCAGAGGTGAAGAAGCCCGGAGCGAGCGTGA |
| | AGGTGAGCTGCAAGGCCAGCGGCTACACCTTTACCAGCCACTGGATCACCTGGGT |
| | GAGGCAAGCCCCTGGACAGCGACTGGAGTGGATGGGCGACATCTACCCCGGCAGC |
| | GGCAACACAAACTACAACGAGAAGTTCCAGGGCAGGGTGACCATCACCGTGGACA |
| | CCAGTGCCAGCACAGCCTACATGGAGCTGAGTAGCCTCAGGAGCGAAGACACCGC |
| | CGTGTACTACTGCGCCAGGGAAAGGGGCGGATTCGACTACTGGGGCCAGGGCACC |
| | CTGGTGACCGTTAGCAGCGCTAGCACCAAGGGCCCCAGCGTGTTCCCTCTGGCCC |
| | CCAGCAGCAAGAGCACCAGCGGCGGAACCGCCGCCCTGGGCTGCCTGGTGAAGGA |
| | CTACTTCCCCGAGCCCGTGACCGTGTCCTGGAACAGCGGCGCTCTGACCAGCGGA |
| | GTGCACACCTTCCCTGCCGTGCTGCAGAGCAGCGGCCTGTACTCCCTGAGCAGCG |
| | TGGTGACCGTGCCCAGCAGCAGCCTGGGCACCCAGACCTACATCTGCAACGTGAA |
| | CCACAAGCCCTCCAACACCAAGGTGGACAAGAAGGTGGAGCCTAAGAGCTGCGAC |
| | AAGACCCACACCTGCCCTCCCTGCCCCGCCCCCGAGCTGCTGGGCGGACCCAGCG |
| | TGTTCCTGTTCCCTCCCAAGCCCAAGGACACCCTGATGATCAGCCGCACCCCCGA |
| | GGTGACCTGCGTGGTGGTGGACGTGAGCCACGAGGACCCCGAGGTGAAGTTCAAC |
| | TGGTACGTGGACGGCGTGGAGGTGCACAACGCCAAGACCAAGCCTCGGGAGGAGC |
| | AGTACAACTCCACCTACCGCGTGGTGAGCGTGCTGACCGTGCTGCACCAGGACTG |
| | GCTGAACGGCAAGGAGTACAAGTGCAAGGTGAGCAACAAGGCCCTGCCCGCTCCC |
| | ATCGAGAAGACCATCAGCAAGGCCAAGGGCCAGCCCCGGGAGCCTCAGGTGTACA |
| | CCCTGCCCCCCAGCCGCGAAGAGATGACCAAGAACCAGGTGAGCCTGACCTGCCT |
| | GGTGAAGGGCTTCTACCCCTCCGACATCGCCGTGGAGTGGGAGAGCAACGGCCAG |
| | CCTGAGAACAACTACAAGACCACCCCTCCCGTGCTGGACAGCGACGGCAGCTTCT |
| | TCCTGTACAGCAAGCTGACCGTGGACAAGTCCCGGTGGCAGCAGGGCAACGTGTT |
| | CAGCTGCAGCGTGATGCACGAGGCCCTGCACAACCACTACACCCAGAAGAGCCTG |
| | AGCCTGAGCCCCGGATAG |
| 150 | (X)_{n = 42-120} |
| | CAGGTGCAGCTCCAGCAGCCCGGAGCGGAGCTGGTTAAGCCTGGCGCAAGCGTGA |
| | AGATGAGTTGCAAGGCCAGCGGCTACACCTTCACCAGCCACTGGATCACCTGGGT |
| | GAAGCAGAGGCCGGGACAGGGCCTGGAGTGGATCGGCGACATCTACCCCGGCAGC |
| | GGCAACACCAACTACAACGAGAAGTTCAAGAGTAAGGCCACACTGACCGTGGACA |
| | CCAGTAGCAGCACCGCCTACATGCAGCTGAGCAGCCTGACCAGCGAGGACAGCGC |
| | CGACTACTACTGTGCCCGCGAGAGGGGCGGATTCGACTATTGGGGCCAAGGCACC |
| | ACCTTGACCGTCAGCTCTGCTAGCACCAAGGGCCCCAGCGTGTTTCCTCTCGCTC |
| | CCTGCAGCCGGAGCACATCCGAGAGCACCGCTGCTCTGGGCTGTCTCGTGAAGGA |
| | CTACTTCCCTGAACCCGTCACCGTCAGCTGGAATAGCGGCGCCCTGACATCCGGC |
| | GTCCACACATTCCCCGCTGTCCTGCAGAGCAGCGGCCTGTACAGCCTGAGCTCCG |
| | TGGTCACCGTGCCTAGCAGCAGCCTGGGAACAAAGACCTACACCTGCAACGTGGA |
| | CCATAAGCCCTCCAACACCAAGGTGGACAAGCGGGTGGAATCCAAGTATGGACCC |
| | CCCTGTCCTCCTTGCCCTGCTCCTGAATTTCTCGGAGGCCCCTCCGTCTTCCTGT |
| | TTCCCCCCAAGCCCAAGGACACCCTGATGATCTCCCGGACACCCGAAGTCACCTG |
| | CGTCGTGGTGGATGTCAGCCAGGAAGATCCCGAGGTGCAGTTCAACTGGTACGTG |
| | GACGGAGTGGAGGTGCATAACGCCAAAACCAAGCCCAGGGAAGAGCAGTTCAACA |
| | GCACCTATCGGGTCGTGTCCGTGCTCACCGTCCTGCATCAGGATTGGCTCAACGG |
| | CAAGGAGTACAAGTGCAAGGTGTCCAACAAGGGCCTGCCCTCCTCCATCGAGAAG |
| | ACCATCTCCAAGGCTAAGGGCCAACCTCGGGAGCCCCAAGTGTATACCCTCCCTC |
| | CCAGCCAGGAGGAGATGACCAAGAATCAAGTGAGCCTGACCTGCCTCGTGAAGGG |
| | ATTTTACCCCTCCGACATCGCTGTGGAATGGGAAAGCAATGGCCAACCTGAGAAC |
| | AACTACAAGACCACACCCCCCGTGCTGGACTCCGATGGCTCCTTCTTCCTGTACA |
| | GCAGGCTGACCGTGGACAAATCCCGGTGGCAAGAGGGAAACGTGTTCAGCTGCTC |
| | CGTGATGCACGAGGCTCTCCACAACCACTACACCCAGAAGAGCCTCTCCCTGAGC |
| | CTCGGCTAG |
| 151 | (X)_{n = 42-120} |
| | CAGGTGCAGCTGGTGCAGAGCGGAGCAGAGGTGAAGAAGCCCGGAGCGAGCGTGA |
| | AGGTGAGCTGCAAGGCCAGCGGCTACACCTTTACCAGCCACTGGATCAGCTGGGT |
| | GAGGCAAGCCCCTGGACAGGGCCTGGAGTGGATGGGCGACATCTACCCCGGCAGC |
| | GGCAACACAAACTACAACGAGAAGCTGCAGGGCAGGGTGACCATGACCGTGGACA |
| | CCAGTACCAGCACAGCCTACATGGAGCTGAGGAGCCTCAGGAGCGACGACACCGC |
| | CGTGTACTACTGCGCCAGGGAAAGGGGCGGATTCGACTACTGGGGCCAGGGCACC |
| | CTGGTGACCGTTAGCAGCGCTAGCACCAAGGGCCCCAGCGTGTTTCCTCTCGCTC |
| | CCTGCAGCCGGAGCACATCCGAGAGCACCGCTGCTCTGGGCTGTCTCGTGAAGGA |
| | CTACTTCCCTGAACCCGTCACCGTCAGCTGGAATAGCGGCGCCCTGACATCCGGC |
| | GTCCACACATTCCCCGCTGTCCTGCAGAGCAGCGGCCTGTACAGCCTGAGCTCCG |
| | TGGTCACCGTGCCTAGCAGCAGCCTGGGAACAAAGACCTACACCTGCAACGTGGA |
| | CCATAAGCCCTCCAACACCAAGGTGGACAAGCGGGTGGAATCCAAGTATGGACCC |
| | CCCTGTCCTCCTTGCCCTGCTCCTGAATTTCTCGGAGGCCCCTCCGTCTTCCTGT |
| | TTCCCCCCAAGCCCAAGGACACCCTGATGATCTCCCGGACACCCGAAGTCACCTG |
| | CGTCGTGGTGGATGTCAGCCAGGAAGATCCCGAGGTGCAGTTCAACTGGTACGTG |
| | GACGGAGTGGAGGTGCATAACGCCAAAACCAAGCCCAGGGAAGAGCAGTTCAACA |
| | GCACCTATCGGGTCGTGTCCGTGCTCACCGTCCTGCATCAGGATTGGCTCAACGG |
| | CAAGGAGTACAAGTGCAAGGTGTCCAACAAGGGCCTGCCCTCCTCCATCGAGAAG |
| | ACCATCTCCAAGGCTAAGGGCCAACCTCGGGAGCCCCAAGTGTATACCCTCCCTC |
| | CCAGCCAGGAGGAGATGACCAAGAATCAAGTGAGCCTGACCTGCCTCGTGAAGGG |
| | ATTTTACCCCTCCGACATCGCTGTGGAATGGGAAAGCAATGGCCAACCTGAGAAC |
| | AACTACAAGACCACACCCCCCGTGCTGGACTCCGATGGCTCCTTCTTCCTGTACA |
| | GCAGGCTGACCGTGGACAAATCCCGGTGGCAAGAGGGAAACGTGTTCAGCTGCTC |
| | CGTGATGCACGAGGCTCTCCACAACCACTACACCCAGAAGAGCCTCTCCCTGAGC |
| | CTCGGCTAG |
| 152 | (X)_{n = 42-120} |
| | CAGGTGCAGCTGGTGCAGAGCGGAGCAGAGGTGAAGAAGCCCGGAGCGAGCGTGA |
| | AGGTGAGCTGCAAGGCCAGCGGCTACACCTTTACCAGCCACTGGATCACCTGGGT |
| | GAGGCAAGCCCCTGGACAGGGCCTGGAGTGGATGGGCGACATCTACCCCGGCAGC |
| | GGCAACACAAACTACAACGAGAAGTTCCAGGGCAGGGTGACCTTGACCGTGGACA |
| | CCAGTATCAGCACAGCCTACATGGAGCTGAGTAGACTCAGGAGCGACGACACCGT |
| | CGTGTACTACTGCGCCAGGGAAAGGGGCGGATTCGACTACTGGGGCCAGGGCACC |
| | CTGGTGACCGTTAGCAGCGCTAGCACCAAGGGCCCCAGCGTGTTTCCTCTCGCTC |
| | CCTGCAGCCGGAGCACATCCGAGAGCACCGCTGCTCTGGGCTGTCTCGTGAAGGA |
| | CTACTTCCCTGAACCCGTCACCGTCAGCTGGAATAGCGGCGCCCTGACATCCGGC |
| | GTCCACACATTCCCCGCTGTCCTGCAGAGCAGCGGCCTGTACAGCCTGAGCTCCG |
| | TGGTCACCGTGCCTAGCAGCAGCCTGGGAACAAAGACCTACACCTGCAACGTGGA |
| | CCATAAGCCCTCCAACACCAAGGTGGACAAGCGGGTGGAATCCAAGTATGGACCC |
| | CCCTGTCCTCCTTGCCCTGCTCCTGAATTTCTCGGAGGCCCCTCCGTCTTCCTGT |
| | TTCCCCCCAAGCCCAAGGACACCCTGATGATCTCCCGGACACCCGAAGTCACCTG |
| | CGTCGTGGTGGATGTCAGCCAGGAAGATCCCGAGGTGCAGTTCAACTGGTACGTG |
| | GACGGAGTGGAGGTGCATAACGCCAAAACCAAGCCCAGGGAAGAGCAGTTCAACA |
| | GCACCTATCGGGTCGTGTCCGTGCTCACCGTCCTGCATCAGGATTGGCTCAACGG |
| | CAAGGAGTACAAGTGCAAGGTGTCCAACAAGGGCCTGCCCTCCTCCATCGAGAAG |
| | ACCATCTCCAAGGCTAAGGGCCAACCTCGGGAGCCCCAAGTGTATACCCTCCCTC |
| | CCAGCCAGGAGGAGATGACCAAGAATCAAGTGAGCCTGACCTGCCTCGTGAAGGG |
| | ATTTTACCCCTCCGACATCGCTGTGGAATGGGAAAGCAATGGCCAACCTGAGAAC |
| | AACTACAAGACCACACCCCCCGTGCTGGACTCCGATGGCTCCTTCTTCCTGTACA |
| | GCAGGCTGACCGTGGACAAATCCCGGTGGCAAGAGGGAAACGTGTTCAGCTGCTC |
| | CGTGATGCACGAGGCTCTCCACAACCACTACACCCAGAAGAGCCTCTCCCTGAGC |
| | CTCGGCTAG |
| 153 | (X)_{n = 42-120} |
| | CAGGTGCAGCTGGTGCAGAGCGGAGCAGAGGTGAAGAAGCCCGGAGCGAGCGTGA |
| | AGGTGAGCTGCAAGGCCAGCGGCTACACCTTTACCAGCCACTGGATCACCTGGGT |
| | GAGGCAAGCCCCTGGACAGCGACTGGAGTGGATGGGCGACATCTACCCCGGCAGC |
| | GGCAACACAAACTACAACGAGAAGTTCCAGGGCAGGGTGACCATCACCGTGGACA |
| | CCAGTGCCAGCACAGCCTACATGGAGCTGAGTAGCCTCAGGAGCGAAGACACCGC |
| | CGTGTACTACTGCGCCAGGGAAAGGGGCGGATTCGACTACTGGGGCCAGGGCACC |
| | CTGGTGACCGTTAGCAGCGCTAGCACCAAGGGCCCCAGCGTGTTTCCTCTCGCTC |
| | CCTGCAGCCGGAGCACATCCGAGAGCACCGCTGCTCTGGGCTGTCTCGTGAAGGA |
| | CTACTTCCCTGAACCCGTCACCGTCAGCTGGAATAGCGGCGCCCTGACATCCGGC |
| | GTCCACACATTCCCCGCTGTCCTGCAGAGCAGCGGCCTGTACAGCCTGAGCTCCG |
| | TGGTCACCGTGCCTAGCAGCAGCCTGGGAACAAAGACCTACACCTGCAACGTGGA |
| | CCATAAGCCCTCCAACACCAAGGTGGACAAGCGGGTGGAATCCAAGTATGGACCC |
| | CCCTGTCCTCCTTGCCCTGCTCCTGAATTTCTCGGAGGCCCCTCCGTCTTCCTGT |
| | TTCCCCCCAAGCCCAAGGACACCCTGATGATCTCCCGGACACCCGAAGTCACCTG |
| | CGTCGTGGTGGATGTCAGCCAGGAAGATCCCGAGGTGCAGTTCAACTGGTACGTG |
| | GACGGAGTGGAGGTGCATAACGCCAAAACCAAGCCCAGGGAAGAGCAGTTCAACA |
| | GCACCTATCGGGTCGTGTCCGTGCTCACCGTCCTGCATCAGGATTGGCTCAACGG |
| | CAAGGAGTACAAGTGCAAGGTGTCCAACAAGGGCCTGCCCTCCTCCATCGAGAAG |
| | ACCATCTCCAAGGCTAAGGGCCAACCTCGGGAGCCCCAAGTGTATACCCTCCCTC |
| | CCAGCCAGGAGGAGATGACCAAGAATCAAGTGAGCCTGACCTGCCTCGTGAAGGG |
| | ATTTTACCCCTCCGACATCGCTGTGGAATGGGAAAGCAATGGCCAACCTGAGAAC |
| | AACTACAAGACCACACCCCCCGTGCTGGACTCCGATGGCTCCTTCTTCCTGTACA |
| | GCAGGCTGACCGTGGACAAATCCCGGTGGCAAGAGGGAAACGTGTTCAGCTGCTC |
| | CGTGATGCACGAGGCTCTCCACAACCACTACACCCAGAAGAGCCTCTCCCTGAGC |
| | CTCGGCTAG |
| 154 | (X)_{n = 42-120} |
| | GACATCCAGATGACCCAGACCACCAGCAGCCTGAGCGCCAGCCTGGGCGACAGGG |
| | TGACCATCAACTGCAGGGCCAGCCAGGACATAAGCAACTTCCTGAACTGGTATCA |
| | ACAGAAGCCCGACGGCACCCTGAAGCTGCTGATCTACTACACCAGCGGCCTGCAC |
| | AGCGGCGTGCCCAGTAGGTTCAGCGGTAGCGGCTCCGGCACCGATTATAGCCTGA |
| | CTATCAGCAACCTGGAGGAAGAGGATATCGCCACCTACTTCTGCCAGCAGGACAA |
| | CAACCACCCCTACACCTTTGGCAGCGGCACCAAGTTGGAGATCAAGCGGACCGTG |
| | GCCGCCCCCAGCGTGTTCATCTTCCCTCCCAGCGACGAGCAGCTGAAGTCTGGCA |
| | CCGCCAGCGTGGTGTGCCTGCTGAACAACTTCTACCCCCGCGAGGCCAAGGTGCA |
| | GTGGAAGGTGGACAACGCCCTGCAGAGCGGCAACAGCCAGGAGAGCGTGACCGAG |
| | CAGGACTCCAAGGACAGCACCTACAGCCTGAGCAGCACCCTGACCCTGAGCAAGG |
| | CCGACTACGAGAAGCACAAGGTGTACGCCTGCGAGGTGACCCACCAGGGACTGTC |
| | TAGCCCCGTGACCAAGAGCTTCAACCGGGGCGAGTGCTAA |
| 155 | (X)_{n = 42-120} |
| | GACATCCAGATGACCCAGAGCCCCAGCTCCCTGAGCGCCAGCGTGGGCGACAGGG |
| | TGACCATCACGTGCAGGGCCAGCCAGGACATCAGCAACTTCCTGAACTGGTATCA |
| | ACAGAAGCCCGAAGGCACCCTGAAGCTGCTTATATACTACACCAGCGGCCTGCAC |
| | AGCGGCGTGCCCAGCCGATTCAGCGGCAGCGGGAGTGGCACCGACTTCACCCTTA |
| | CAATCAGCAGTCTGCAGCCCGAGGACGTGGCTACCTACTACTGCCAGCAGGATAA |
| | CAACCACCCCTACACCTTCGGCCAGGGCACTAAGCTGGAGATCAAGCGGACCGTG |
| | GCCGCCCCCAGCGTGTTCATCTTCCCTCCCAGCGACGAGCAGCTGAAGTCTGGCA |
| | CCGCCAGCGTGGTGTGCCTGCTGAACAACTTCTACCCCCGCGAGGCCAAGGTGCA |
| | GTGGAAGGTGGACAACGCCCTGCAGAGCGGCAACAGCCAGGAGAGCGTGACCGAG |
| | CAGGACTCCAAGGACAGCACCTACAGCCTGAGCAGCACCCTGACCCTGAGCAAGG |
| | CCGACTACGAGAAGCACAAGGTGTACGCCTGCGAGGTGACCCACCAGGGACTGTC |
| | TAGCCCCGTGACCAAGAGCTTCAACCGGGGCGAGTGCTAA |
| 156 | (X)_{n = 42-120} |
| | GACATCCAGATGACCCAGAGCCCCAGCTCCCTGAGCGCCAGCGTGGGCGACAGGG |
| | TGACCATCACGTGCCAGGCCAGCCAGGACATCAGCAACTTCCTGAACTGGTATCA |
| | ACAGAAGCCCGGAAAGACCCTGAAGCTGCTTATATACTACACCAGCGGCCTGCAC |
| | ACCGGCGTGCCCAGCCGATTCAGCGGCAGCGGGAGTGGCACCGACTTCACCTTTA |
| | CAATCAGCAGTCTGCAGCCCGAGGACATTGCTACCTACTACTGCCAGCAGGATAA |
| | CAACCACCCCTACACCTTCGGCCAGGGCACTAAGCTGGAGATCAAGCGGACCGTG |
| | GCCGCCCCCAGCGTGTTCATCTTCCCTCCCAGCGACGAGCAGCTGAAGTCTGGCA |
| | CCGCCAGCGTGGTGTGCCTGCTGAACAACTTCTACCCCCGCGAGGCCAAGGTGCA |
| | GTGGAAGGTGGACAACGCCCTGCAGAGCGGCAACAGCCAGGAGAGCGTGACCGAG |
| | CAGGACTCCAAGGACAGCACCTACAGCCTGAGCAGCACCCTGACCCTGAGCAAGG |
| | CCGACTACGAGAAGCACAAGGTGTACGCCTGCGAGGTGACCCACCAGGGACTGTC |
| | TAGCCCCGTGACCAAGAGCTTCAACCGGGGCGAGTGCTAA |
| 157 | (X)_{n = 42-120} |
| | GACATCCAGATGACCCAGAGCCCCAGCTCCCTGAGCGCCAGCGTGGGCGACAGGG |
| | TGACCATCACGTGCAGGGCCAGCCAGGACATCAGCAACTTCCTGAACTGGTATCA |
| | ACAGAAGCCCGGAAAGGCTCCGAAGCTGCTTATATACTACACCAGCGGCCTGCAC |
| | AGCGGCGTGCCCAGCCGATTCAGCGGCAGCGGGAGTGGCACCGACTTCACCCTTA |
| | CAATCAGCAGTCTGCAGCCCGAGGACTTCGCTACCTACTACTGCCAGCAGGATAA |
| | CAACCACCCCTACACCTTCGGCCAGGGCACTAAGCTGGAGATCAAGCGGACCGTG |
| | GCCGCCCCCAGCGTGTTCATCTTCCCTCCCAGCGACGAGCAGCTGAAGTCTGGCA |
| | CCGCCAGCGTGGTGTGCCTGCTGAACAACTTCTACCCCCGCGAGGCCAAGGTGCA |
| | GTGGAAGGTGGACAACGCCCTGCAGAGCGGCAACAGCCAGGAGAGCGTGACCGAG |
| | CAGGACTCCAAGGACAGCACCTACAGCCTGAGCAGCACCCTGACCCTGAGCAAGG |
| | CCGACTACGAGAAGCACAAGGTGTACGCCTGCGAGGTGACCCACCAGGGACTGTC |
| | TAGCCCCGTGACCAAGAGCTTCAACCGGGGCGAGTGCTAA |

(X)_{n = 42-120} is a leader sequence

In some embodiments, the anti-CD122 antibody comprise full length antibody chain polypeptides that are encoded by a HC nucleic acid sequence and a LC nucleic acid sequence. In some embodiments, the anti-CD122 antibody comprises a full length antibody chain that is encoded by a HC nucleic acid sequence comprising SEQ ID NO: 146 and a LC nucleic acid sequence comprising SEQ ID NO: 154. In some embodiments, the anti-CD122 antibody comprises a full length antibody chain that is encoded by a HC nucleic acid sequence comprising SEQ ID NO: 147 and a LC nucleic acid sequence comprising SEQ ID NO: 155. In some embodiments, the anti-CD122 antibody comprises a full length antibody chain that is encoded by a HC nucleic acid sequence comprising SEQ ID NO: 147 and a LC nucleic acid sequence comprising SEQ ID NO: 156. In some embodiments, the anti-CD122 antibody comprises a full length antibody chain that is encoded by a HC nucleic acid sequence comprising SEQ ID NO: 147 and a LC nucleic acid sequence comprising SEQ ID NO: 157. In some embodiments, the anti-CD122 antibody comprises a full length antibody chain that is encoded by a HC nucleic acid sequence comprising SEQ ID NO: 148 and a LC nucleic acid sequence comprising SEQ ID NO: 155. In some embodiments, the anti-CD122 antibody comprises a full length antibody chain that is encoded by a HC nucleic acid sequence comprising SEQ ID NO: 148 and a LC nucleic acid sequence comprising SEQ ID NO: 156. In some embodiments, the anti-CD122 antibody comprises a full length antibody chain that is encoded by a HC nucleic acid sequence comprising SEQ ID NO: 148 and a LC nucleic acid sequence comprising SEQ ID NO: 157. In some embodiments, the anti-CD122 antibody comprises a full length antibody chain that is encoded by a HC nucleic acid sequence comprising SEQ ID NO: 149 and a LC nucleic acid sequence comprising SEQ ID NO: 155. In some embodiments, the anti-CD122 antibody comprises a full length antibody chain that is encoded by a HC nucleic acid sequence comprising SEQ ID NO: 149 and a LC nucleic acid sequence comprising SEQ ID NO: 156. In some embodiments, the anti-CD122 antibody comprises a full length antibody chain that is encoded by a HC nucleic acid sequence comprising SEQ ID NO: 149 and a LC nucleic acid sequence comprising SEQ ID NO: 157. In some embodiments, the anti-CD122 antibody comprises a full length antibody chain that is encoded by a HC nucleic acid sequence comprising SEQ ID NO: 150 and a LC nucleic acid sequence comprising SEQ ID NO: 154. In some embodiments, the anti-CD122 antibody comprises a full length antibody chain that is encoded by a HC nucleic acid sequence comprising SEQ ID NO: 151 and a LC nucleic acid sequence comprising SEQ ID NO: 155. In some embodiments, the anti-CD122 antibody comprises a full length antibody chain that is encoded by a HC nucleic acid sequence comprising SEQ ID NO: 151 and a LC nucleic acid sequence comprising SEQ ID NO: 156. In some embodiments, the anti-CD122 antibody comprises a full length antibody chain that is encoded by a HC nucleic acid sequence comprising SEQ ID NO: 151 and a LC nucleic acid sequence comprising SEQ ID NO: 157. In some embodiments, the anti-CD122 antibody comprises a full length antibody chain that is encoded by a HC nucleic acid sequence comprising SEQ ID NO: 152 and a LC nucleic acid sequence comprising SEQ ID NO: 155. In some embodiments, the anti-CD122 antibody comprises a full length antibody chain that is encoded by a HC nucleic acid sequence comprising SEQ ID NO: 152 and a LC nucleic acid sequence comprising SEQ ID NO: 156. In some embodiments, the anti-CD122 antibody comprises a full length antibody chain that is encoded by a HC nucleic acid sequence comprising SEQ ID NO: 152 and a LC nucleic acid sequence comprising SEQ ID NO: 157. In some embodiments, the anti-CD122 antibody comprises a full length antibody chain that is encoded by a HC nucleic acid sequence comprising SEQ ID NO: 153 and a LC nucleic acid sequence comprising SEQ ID NO: 155. In some embodiments, the anti-CD122 antibody comprises a full length antibody chain that is encoded by a HC nucleic acid sequence comprising SEQ ID NO: 153 and a LC nucleic acid sequence comprising SEQ ID NO: 156. In some embodiments, the anti-CD122 antibody comprises a full length antibody chain that is encoded by a HC nucleic acid sequence comprising SEQ ID NO: 153 and a LC nucleic acid sequence comprising SEQ ID NO: 157.

In some embodiments, the anti-CD122 antibody heavy chain and the anti-CD122 antibody light chain have a leader sequence near an amino-terminal region of each polypeptide. In some embodiments, the leader sequence is N-terminal to an anti-CD122 antibody heavy chain sequence. In some embodiments, the leader sequence is N-terminal to an anti-CD122 antibody heavy chain sequence provided in Table 9. In some embodiments, the leader sequence is N-terminal to an anti-CD122 antibody light chain sequence. In some embodiments, the leader sequence is N-terminal to an anti-CD122 antibody light chain sequence provided in Table 10. In some embodiments, (X)ₙ₌₁₄₋₄₀ is used to represent amino acids of a leader sequence at the amino-terminal end of an anti-CD122 antibody heavy chain sequence. In some embodiments, (X)ₙ₌₁₄₋₄₀ is used to represent amino acids of a leader sequence at the amino-terminal end of an anti-CD122 antibody light chain sequence. In some embodiments, the leader sequence is a length of between 14-40 amino acids. In some embodiments, the leader sequence is a length of between 15-35 amino acids. In some embodiments, the leader sequence of an anti-CD122 antibody heavy chain and an anti-CD122 antibody light chain paired to form an anti-CD122 antibody are identical to each other. In some embodiments, the leader sequence of an anti-CD122 antibody heavy chain and an anti-CD122 antibody light chain paired to form an anti-CD122 antibody are not identical to each other. In some embodiments, the leader sequence comprises an amino acid sequence listed in Table 12. In some embodiments, the anti-CD122 antibody heavy chain contains an additional amino acid sequence N-terminal to the leader sequence. In some embodiments, the anti-CD122 antibody heavy chain contains an additional amino acid sequence immediately after the leader sequence. In some embodiments, the additional amino acid sequence immediately after the leader sequence is before a start of a heavy chain variable domain. In some embodiments, the anti-CD122 antibody light chain contains an additional amino acid sequence N-terminal to the leader sequence. In some embodiments, the anti-CD122 antibody light chain contains an additional amino acid sequence immediately after the leader sequence. In some embodiments, the additional amino acid sequence immediately after the leader sequence is before a start of a light chain variable domain. In some embodiments, the leader sequence used N-terminal to the anti-CD122 antibody heavy chain sequence comprises a leader sequence comprising SEQ ID NO: 158. In some embodiments, the leader sequence used N-terminal to the anti-CD122 antibody heavy chain sequence comprises a leader sequence comprising SEQ ID NO: 159. In some embodiments, the leader sequence used N-terminal to the anti-CD122 antibody heavy chain sequence comprises a leader sequence comprising SEQ ID NO: 160. In some embodiments, the leader sequence used N-terminal to the anti-CD122 antibody heavy chain sequence comprises a leader sequence comprising SEQ ID NO: 161.

In some embodiments, the leader sequence used N-terminal to the anti-CD122 antibody light chain sequence comprises a leader sequence comprising SEQ ID NO:158. In some embodiments, the leader sequence used N-terminal to the anti-CD122 antibody light chain sequence comprises a leader sequence comprising SEQ ID NO: 159. In some embodiments, the leader sequence used N-terminal to the anti-CD122 antibody light chain sequence comprises a leader sequence comprising SEQ ID NO: 160. In some embodiments, the leader sequence used N-terminal to the anti-CD122 antibody light chain sequence comprises a leader sequence comprising SEQ ID NO:161.

In some embodiments, the anti-CD122 antibody comprises full length antibody chain polypeptides that are encoded by a HC nucleic acid sequence and a LC nucleic acid sequence each having a nucleic acid sequence encoding a leader sequence near a 5′-end of a nucleic acid encoding the HC or near a 5′-end of a nucleic acid encoding the LC. In some embodiments, the nucleic acid sequence encoding a leader sequence is 5′ to an anti-CD122 antibody heavy chain coding nucleic acid sequence. In some embodiments, the anti-CD122 antibody heavy chain coding nucleic acid sequence that has the nucleic acid sequence encoding a leader sequence is provided in Table 11. In some embodiments, the nucleic acid sequence encoding a leader sequence is 5′ to an anti-CD122 antibody light chain coding nucleic acid sequence. In some embodiments, the anti-CD122 antibody light chain coding nucleic acid sequence that has the nucleic acid sequence encoding a leader sequence is provided in Table 11. In some embodiments, (X)ₙ₌₄₂₋₁₂₀ is used to represent nucleotides of a leader sequence at the 5′-end of an anti-CD122 antibody heavy chain coding nucleic acid sequence. In some embodiments, (X)ₙ₌₄₂₋₁₂₀ is used to represent nucleotides of a leader sequence at the 5′-end of an anti-CD122 antibody light chain coding nucleic acid sequence. In some embodiments, the leader sequence is a length of between 42-120 nucleotides. In some embodiments, the leader sequence is a length of between 45-105 nucleotides. In some embodiments, the leader sequence of an anti-CD122 antibody heavy chain encoding sequence and the leader sequence of an anti-CD122 antibody light chain encoding sequence of an anti-CD122 antibody are identical to each other. In some embodiments, the leader sequence of an anti-CD122 antibody heavy chain encoding sequence and the leader sequence of an anti-CD122 antibody light chain encoding sequence of an anti-CD122 antibody are not identical to each other. In some embodiments, the leader sequence encodes an amino acid sequence listed in Table 12. In some embodiments, the anti-CD122 antibody heavy chain encoding sequence contains an additional nucleotide sequence at the 5′-end of the leader sequence. In some embodiments, the anti-CD122 antibody heavy chain encoding sequence contains an additional nucleotide sequence immediately at the 3′-end of the leader sequence. In some embodiments, the anti-CD122 antibody light chain encoding sequence contains an additional nucleotide sequence at the 5′-end of the leader sequence. In some embodiments, the anti-CD122 antibody light chain encoding sequence contains an additional nucleotide sequence immediately at the 3′-end of the leader sequence. In some embodiments, the leader sequence at the 5′-end of the anti-CD122 antibody heavy chain encoding sequence encodes an amino acid leader sequence of SEQ ID NO: 158. In some embodiments, the leader sequence at the 5′-end of the anti-CD122 antibody heavy chain encoding sequence encodes an amino acid leader sequence of SEQ ID NO: 159. In some embodiments, the leader sequence at the 5′-end of the anti-CD122 antibody heavy chain encoding sequence encodes an amino acid leader sequence of SEQ ID NO: 160. In some embodiments, the leader sequence at the 5′-end of the anti-CD122 antibody heavy chain encoding sequence encodes an amino acid leader sequence of SEQ ID NO: 161. In some embodiments, the leader sequence at the 5′-end of the anti-CD122 antibody light chain encoding sequence encodes an amino acid leader sequence of SEQ ID NO: 158. In some embodiments, the leader sequence at the 5′-end of the anti-CD122 antibody light chain encoding sequence encodes an amino acid leader sequence of SEQ ID NO: 159. In some embodiments, the leader sequence at the 5′-end of the anti-CD122 antibody light chain encoding sequence encodes an amino acid leader sequence of SEQ ID NO: 160. In some embodiments, the leader sequence at the 5′-end of the anti-CD122 antibody light chain encoding sequence encodes an amino acid leader sequence of SEQ ID NO: 161.

TABLE 12
Leader Sequences for use with
heavy chains and light chains

| SEQ ID | |
|---|---|
| NO: | Leader Sequence |
| 158 | MDPKGSLSWRILLFLSLAFELSYG |
| 159 | METDTLLLWVLLLWVPGSTG |
| 160 | MGWSLILLFLVAVATRVHS |
| 161 | MRVPAQLLGLLLLWLPGARC |

In some embodiments, an HC polypeptide and an LC polypeptide are paired together to form an anti-CD122 antibody or an anti-CD122 antibody fragment. In some embodiments, two heavy chains are linked to each other by disulfide bonds and each heavy chain is linked to a light chain by a disulfide bond. In some embodiments, the particular HC polypeptides and particular LC polypeptides paired together to form an anti-CD122 antibody are named from G1-G20 according to Table 13. In some embodiments, G1 is a chimeric anti-CD122 antibody with mouse parental variable domain sequences in a human IgG1 backbone. In some embodiments, G2-10 are humanized anti-CD122 antibodies with mouse-derived CDRs incorporated into human variable domain framework sequences that have been optimized to retain affinity to CD122 in a human IgG1 backbone. In some embodiments, G11 is a chimeric anti-CD122 antibody with mouse parental variable domain sequences in a human IgG4 backbone. In some embodiments, G12-20 are humanized anti-CD122 antibodies with mouse-derived CDRs incorporated into human variable domain framework sequences that have been optimized to retain affinity to CD122 in a human IgG4 backbone.

TABLE 13
Antibody names with heavy chain/light
chain sequence combinations

| | HC polypeptide | LC polypeptide |
|---|---|---|
| Antibody name | SEQ ID NO: | SEQ ID NO: |
| G1 | 134 | 142 |
| G2 | 135 | 143 |
| G3 | 135 | 144 |
| G4 | 135 | 145 |
| G5 | 136 | 143 |
| G6 | 136 | 144 |
| G7 | 136 | 145 |
| G8 | 137 | 143 |
| G9 | 137 | 144 |
| G10 | 137 | 145 |
| G11 | 138 | 142 |
| G12 | 139 | 143 |
| G13 | 139 | 144 |
| G14 | 139 | 145 |
| G15 | 140 | 143 |
| G16 | 140 | 144 |
| G17 | 140 | 145 |
| G18 | 141 | 143 |
| G19 | 141 | 144 |
| G20 | 141 | 145 |

In some embodiments, the anti-CD122 antibody CDR sequences can be defined by an antibody numbering scheme. In embodiments, the antibody numbering scheme is Kabat. In embodiments, the antibody numbering scheme is IMGT. In embodiments, the antibody numbering scheme is AbM. In embodiments, the antibody numbering scheme is Chothia. In embodiments, the antibody numbering scheme is Contact. Table 14 lists CDR sequences for anti-CD122 antibodies and antigen binding fragments thereof according to various antibody numbering schemes. In Table 14, the HCDR1, HCDR2, and HCDR3 sequences defined according to a specific antibody numbering scheme are found in an VH region of a heavy chain, wherein the heavy chain sequence according to a representative SEQ ID NO. is listed in Table 9. In Table 14, the LCDR1, LCDR2, and LCDR3 sequences defined according to a specific antibody numbering scheme are found in an VL region of a light chain, wherein the light chain sequence according to a representative SEQ ID NO. is listed in Table 10.

TABLE 14
CDR sequences for anti-CD122 antibody heavy chain variable domains and light
chain variable domains contained within a heavy chain or a light chain

| SEQ | | | | | | | |
|---|---|---|---|---|---|---|---|
| ID | | | | | | | |
| NO. of | | | | | | | |
| heavy | | | | | | | |
| chain | | | | | | | |
| from | | SEQ | | SEQ | | SEQ | |
| Table | Numbering | ID | | ID | | ID | |
| 9 | Scheme | NO: | HCDR1 Sequence | NO: | HCDR2 Sequence | NO: | HCDR3 Sequence |
| 134 | Kabat | 162 | SHWIT | 169 | DIYPGSGNTNYNEKFKS | 176 | ERGGFDY |
| or | IMGT | 7 | GYTFTSHW | 18 | IYPGSGNT | 30 | ARERGGFDY |
| 138 | AbM | 163 | GYTFTSHWIT | 170 | DIYPGSGNTN | 176 | ERGGFDY |
| | Chothia | 164 | GYTFTSH | 171 | YPGSGN | 176 | ERGGFDY |
| | Contact | 165 | TSHWIT | 172 | WIGDIYPGSGNTN | 177 | ARERGGFD |
| 135 | Kabat | 166 | SHWIS | 173 | DIYPGSGNTNYNEKLQG | 176 | ERGGFDY |
| or | IMGT | 7 | GYTFTSHW | 18 | IYPGSGNT | 30 | ARERGGFDY |
| 139 | AbM | 167 | GYTFTSHWIS | 170 | DIYPGSGNTN | 176 | ERGGFDY |
| | Chothia | 164 | GYTFTSH | 171 | YPGSGN | 176 | ERGGFDY |
| | Contact | 168 | TSHWIS | 174 | WMGDIYPGSGNTN | 177 | ARERGGFD |
| 136 | Kabat | 162 | SHWIT | 175 | DIYPGSGNTNYNEKFQG | 176 | ERGGFDY |
| or | IMGT | 7 | GYTFTSHW | 18 | IYPGSGNT | 30 | ARERGGFDY |
| 137 | AbM | 163 | GYTFTSHWIT | 170 | DIYPGSGNTN | 176 | ERGGFDY |
| or | Chothia | 164 | GYTFTSH | 171 | YPGSGN | 176 | ERGGFDY |
| 140 | Contact | 165 | TSHWIT | 174 | WMGDIYPGSGNTN | 177 | ARERGGFD |
| or | | | | | | | |
| 141 | | | | | | | |
| SEQ | | | | | | | |
| ID | | | | | | | |
| NO. of | | | | | | | |
| light | | | | | | | |
| chain | | | | | | | |
| from | | SEQ | | SEQ | | SEQ | |
| Table | Numbering | ID | | ID | | ID | |
| 10 | Scheme | NO: | LCDR1 Sequence | NO | LCDR2 Sequence | NO: | LCDR3 Sequence |
| 142 | Kabat | 178 | RASQDISNFLN | 181 | YTSGLHS | 62 | QQDNNHPYT |
| or | IMGT | 43 | QDISNF | | YTS | 62 | QQDNNHPYT |
| 143 | AbM | 178 | RASQDISNFLN | 181 | YTSGLHS | 62 | QQDNNHPYT |
| or | Chothia | 178 | RASQDISNFLN | 181 | YTSGLHS | 62 | QQDNNHPYT |
| 145 | Contact | 179 | SNFLNWY | 182 | LLIYYTSGLH | 184 | QQDNNHPY |
| 144 | Kabat | 180 | QASQDISNFLN | 183 | YTSGLHT | 62 | QQDNNHPYT |
| | IMGT | 43 | QDISNF | | YTS | 62 | QQDNNHPYT |
| | AbM | 180 | QASQDISNFLN | 183 | YTSGLHT | 62 | QQDNNHPYT |
| | Chothia | 180 | QASQDISNFLN | 183 | YTSGLHT | 62 | QQDNNHPYT |
| | Contact | 179 | SNFLNWY | 182 | LLIYYTSGLH | 184 | QQDNNHPY |

In some aspects, anti-CD122 antibodies or antigen binding fragments thereof comprise CDR sequences listed in Table 14. In some embodiments, the anti-CD122 antibody or antigen binding fragment thereof comprises a VH domain comprising HCDR sequences listed in Table 14. In some embodiments, the anti-CD122 antibody or antigen binding fragment thereof comprises a VL domain comprising LCDR sequences listed in Table 14. In some embodiments, the anti-CD122 antibody or antigen binding fragment thereof comprises a VH domain comprising HCDR sequences listed in Table 14 and a VL domain comprising LCDR sequences listed in Table 14. In some embodiments, the anti-CD122 antibody or antigen binding fragment thereof comprises a VH domain comprising an HCDR1 sequence, an HCDR2 sequence, and an HCDR3 sequence listed in Table 14 according to the Kabat numbering scheme. In some embodiments, the anti-CD122 antibody or antigen binding fragment thereof comprises a VH domain comprising an HCDR1 sequence, an HCDR2 sequence, and an HCDR3 sequence listed in Table 14 according to the IMGT numbering scheme. In some embodiments, the anti-CD122 antibody or antigen binding fragment thereof comprises a VH domain comprising an HCDR1 sequence, an HCDR2 sequence, and an HCDR3 sequence listed in Table 14 according to the AbM numbering scheme. In some embodiments, the anti-CD122 antibody or antigen binding fragment thereof comprises a VH domain comprising an HCDR1 sequence, an HCDR2 sequence, and an HCDR3 sequence listed in Table 14 according to the Chothia numbering scheme. In some embodiments, the anti-CD122 antibody or antigen binding fragment thereof comprises a VH domain comprising an HCDR1 sequence, an HCDR2 sequence, and an HCDR3 sequence listed in Table 14 according to the Contact numbering scheme. In some embodiments, the anti-CD122 antibody or antigen binding fragment thereof comprises a VL domain comprising an LCDR1 sequence, an LCDR2 sequence, and an LCDR3 sequence listed in Table 14 according to the Kabat numbering scheme. In some embodiments, the anti-CD122 antibody or antigen binding fragment thereof comprises a VL domain comprising an LCDR1 sequence, an LCDR2 sequence, and an LCDR3 sequence listed in Table 14 according to the IMGT numbering scheme. In some embodiments, the anti-CD122 antibody or antigen binding fragment thereof comprises a VL domain comprising an LCDR1 sequence, an LCDR2 sequence, and an LCDR3 sequence listed in Table 14 according to the AbM numbering scheme. In some embodiments, the anti-CD122 antibody or antigen binding fragment thereof comprises a VL domain comprising an LCDR1 sequence, an LCDR2 sequence, and an LCDR3 sequence listed in Table 14 according to the Chothia numbering scheme. In some embodiments, the anti-CD122 antibody or antigen binding fragment thereof comprises a VL domain comprising an LCDR1 sequence, an LCDR2 sequence, and an LCDR3 sequence listed in Table 14 according to the Contact numbering scheme. In some embodiments, the anti-CD122 antibody or antigen binding fragment thereof comprises a VH domain comprising an HCDR1 sequence, an HCDR2 sequence, and an HCDR3 sequence listed in Table 14 according to the Kabat numbering scheme and a VL domain comprising an LCDR1 sequence, an LCDR2 sequence, and an LCDR3 sequence listed in Table 14 according to the Kabat numbering scheme. In some embodiments, the anti-CD122 antibody or antigen binding fragment thereof comprises a VH domain comprising an HCDR1 sequence, an HCDR2 sequence, and an HCDR3 sequence listed in Table 14 according to the IMGT numbering scheme and a VL domain comprising an LCDR1 sequence, an LCDR2 sequence, and an LCDR3 sequence listed in Table 14 according to the IMGT numbering scheme. In some embodiments, the anti-CD122 antibody or antigen binding fragment thereof comprises a VH domain comprising an HCDR1 sequence, an HCDR2 sequence, and an HCDR3 sequence listed in Table 14 according to the AbM numbering scheme and a VL domain comprising an LCDR1 sequence, an LCDR2 sequence, and an LCDR3 sequence listed in Table 14 according to the AbM numbering scheme. In some embodiments, the anti-CD122 antibody or antigen binding fragment thereof comprises a VH domain comprising an HCDR1 sequence, an HCDR2 sequence, and an HCDR3 sequence listed in Table 14 according to the Chothia numbering scheme and a VL domain comprising an LCDR1 sequence, an LCDR2 sequence, and an LCDR3 sequence listed in Table 14 according to the Chothia numbering scheme. In some embodiments, the anti-CD122 antibody or antigen binding fragment thereof comprises a VH domain comprising an HCDR1 sequence, an HCDR2 sequence, and an HCDR3 sequence listed in Table 14 according to the Contact numbering scheme and a VL domain comprising an LCDR1 sequence, an LCDR2 sequence, and an LCDR3 sequence listed in Table 14 according to the Contact numbering scheme.

In some aspects, anti-CD122 antibodies or antigen binding fragments thereof comprise a VH domain comprising HCDR sequences listed according to an antibody numbering scheme in Table 14 identified by SEQ ID NO. and a VL domain comprising LCDR sequences listed according to an antibody numbering scheme in Table 14 identified by SEQ ID NO. In some embodiments, the anti-CD122 antibody comprises a VH domain comprising HCDR sequences listed according to an antibody numbering scheme in Table 14 identified by SEQ ID NO. and a VL domain comprising LCDR sequences listed according to an antibody numbering scheme in Table 14 identified by SEQ ID NO. In some embodiments, the anti-CD122 antigen binding fragment thereof comprises a VH domain comprising HCDR sequences listed according to an antibody numbering scheme in Table 14 identified by SEQ ID NO. and a VL domain comprising LCDR sequences listed according to an antibody numbering scheme in Table 14 identified by SEQ ID NO. In some embodiments, the anti-CD122 antibody or antigen binding fragment thereof comprises an HCDR1 having the sequence of SEQ ID NO: 7, an HCDR2 having the sequence of SEQ ID NO: 18, an HCDR3 having the sequence of SEQ ID NO: 30, an LCDR1 having the sequence of SEQ ID NO: 43, an LCDR2 having the sequence of YTS, and an LCDR3 having the sequence of SEQ ID NO: 62, according to the IMGT antibody numbering scheme. In some embodiments, the anti-CD122 antibody or antigen binding fragment thereof comprises an HCDR1 having the sequence of SEQ ID NO: 162, an HCDR2 having the sequence of SEQ ID NO: 169, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the Kabat antibody numbering scheme. In some embodiments, the anti-CD122 antibody or antigen binding fragment thereof comprises an HCDR1 having the sequence of SEQ ID NO: 163, an HCDR2 having the sequence of SEQ ID NO: 170, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the AbM antibody numbering scheme. In some embodiments, the anti-CD122 antibody or antigen binding fragment thereof comprises an HCDR1 having the sequence of SEQ ID NO: 164, an HCDR2 having the sequence of SEQ ID NO: 171, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the Chothia antibody numbering scheme. In some embodiments, the anti-CD122 antibody or antigen binding fragment thereof comprises an HCDR1 having the sequence of SEQ ID NO: 165, an HCDR2 having the sequence of SEQ ID NO: 172, an HCDR3 having the sequence of SEQ ID NO: 177, an LCDR1 having the sequence of SEQ ID NO: 179, an LCDR2 having the sequence of SEQ ID NO: 182, and an LCDR3 having the sequence of SEQ ID NO: 184, according to the Contact antibody numbering scheme. In some embodiments, the anti-CD122 antibody or antigen binding fragment thereof comprises an HCDR1 having the sequence of SEQ ID NO: 166, an HCDR2 having the sequence of SEQ ID NO: 173, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the Kabat antibody numbering scheme. In some embodiments, the anti-CD122 antibody or antigen binding fragment thereof comprises an HCDR1 having the sequence of SEQ ID NO: 167, an HCDR2 having the sequence of SEQ ID NO: 170, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the AbM antibody numbering scheme. In some embodiments, the anti-CD122 antibody or antigen binding fragment thereof comprises an HCDR1 having the sequence of SEQ ID NO: 164, an HCDR2 having the sequence of SEQ ID NO: 171, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the Chothia antibody numbering scheme. In some embodiments, the anti-CD122 antibody or antigen binding fragment thereof comprises an HCDR1 having the sequence of SEQ ID NO: 168, an HCDR2 having the sequence of SEQ ID NO: 174, an HCDR3 having the sequence of SEQ ID NO: 177, an LCDR1 having the sequence of SEQ ID NO: 179, an LCDR2 having the sequence of SEQ ID NO: 182, and an LCDR3 having the sequence of SEQ ID NO: 184, according to the Contact antibody numbering scheme. In some embodiments, the anti-CD122 antibody or antigen binding fragment thereof comprises an HCDR1 having the sequence of SEQ ID NO: 166, an HCDR2 having the sequence of SEQ ID NO: 173, an HCDR3 having the sequence of SEQ ID NO: 176, an LCDR1 having the sequence of SEQ ID NO: 180, an LCDR2 having the sequence of SEQ ID NO: 183, and an LCDR3 having the sequence of SEQ ID NO: 62, according to the Kabat antibody numbering scheme. In some embodiments, the anti-CD122 antibody or antigen binding fragment thereof comprises an HCDR1 having the sequence of SEQ ID NO: 167, an HCDR2 having the sequence of SEQ ID NO: 170, an HCDR3 having the sequence of SEQ ID NO: 176, an LCDR1 having the sequence of SEQ ID NO: 180, an LCDR2 having the sequence of SEQ ID NO: 183, and an LCDR3 having the sequence of SEQ ID NO: 62, according to the AbM antibody numbering scheme. In some embodiments, the anti-CD122 antibody or antigen binding fragment thereof comprises an HCDR1 having the sequence of SEQ ID NO: 164, an HCDR2 having the sequence of SEQ ID NO: 171, an HCDR3 having the sequence of SEQ ID NO: 176, an LCDR1 having the sequence of SEQ ID NO: 180, an LCDR2 having the sequence of SEQ ID NO: 183, and an LCDR3 having the sequence of SEQ ID NO: 62, according to the Chothia antibody numbering scheme. In some embodiments, the anti-CD122 antibody or antigen binding fragment thereof comprises an HCDR1 having the sequence of SEQ ID NO: 168, an HCDR2 having the sequence of SEQ ID NO: 174, an HCDR3 having the sequence of SEQ ID NO: 177, an LCDR1 having the sequence of SEQ ID NO: 179, an LCDR2 having the sequence of SEQ ID NO: 182, and an LCDR3 having the sequence of SEQ ID NO: 184, according to the Contact antibody numbering scheme. In some embodiments, the anti-CD122 antibody or antigen binding fragment thereof comprises an HCDR1 having the sequence of SEQ ID NO: 162, an HCDR2 having the sequence of SEQ ID NO: 175, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the Kabat antibody numbering scheme. In some embodiments, the anti-CD122 antibody or antigen binding fragment thereof comprises an HCDR1 having the sequence of SEQ ID NO: 163, an HCDR2 having the sequence of SEQ ID NO: 170, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the AbM antibody numbering scheme. In some embodiments, the anti-CD122 antibody or antigen binding fragment thereof comprises an HCDR1 having the sequence of SEQ ID NO: 164, an HCDR2 having the sequence of SEQ ID NO: 171, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the Chothia antibody numbering scheme. In some embodiments, the anti-CD122 antibody or antigen binding fragment thereof comprises an HCDR1 having the sequence of SEQ ID NO: 165, an HCDR2 having the sequence of SEQ ID NO: 174, an HCDR3 having the sequence of SEQ ID NO: 177, an LCDR1 having the sequence of SEQ ID NO: 179, an LCDR2 having the sequence of SEQ ID NO: 182, and an LCDR3 having the sequence of SEQ ID NO: 184, according to the Contact antibody numbering scheme. In some embodiments, the anti-CD122 antibody or antigen binding fragment thereof comprises an HCDR1 having the sequence of SEQ ID NO: 162, an HCDR2 having the sequence of SEQ ID NO: 175, an HCDR3 having the sequence of SEQ ID NO: 176, an LCDR1 having the sequence of SEQ ID NO: 180, an LCDR2 having the sequence of SEQ ID NO: 183, and an LCDR3 having the sequence of SEQ ID NO: 62, according to the Kabat antibody numbering scheme. In some embodiments, the anti-CD122 antibody or antigen binding fragment thereof comprises an HCDR1 having the sequence of SEQ ID NO: 163, an HCDR2 having the sequence of SEQ ID NO: 170, an HCDR3 having the sequence of SEQ ID NO: 176, an LCDR1 having the sequence of SEQ ID NO: 180, an LCDR2 having the sequence of SEQ ID NO: 183, and an LCDR3 having the sequence of SEQ ID NO: 62, according to the AbM antibody numbering scheme. In some embodiments, the anti-CD122 antibody or antigen binding fragment thereof comprises an HCDR1 having the sequence of SEQ ID NO: 164, an HCDR2 having the sequence of SEQ ID NO: 171, an HCDR3 having the sequence of SEQ ID NO: 176, an LCDR1 having the sequence of SEQ ID NO: 180, an LCDR2 having the sequence of SEQ ID NO: 183, and an LCDR3 having the sequence of SEQ ID NO: 62, according to the Chothia antibody numbering scheme. In some embodiments, the anti-CD122 antibody or antigen binding fragment thereof comprises an HCDR1 having the sequence of SEQ ID NO: 165, an HCDR2 having the sequence of SEQ ID NO: 174, an HCDR3 having the sequence of SEQ ID NO: 177, an LCDR1 having the sequence of SEQ ID NO: 179, an LCDR2 having the sequence of SEQ ID NO: 182, and an LCDR3 having the sequence of SEQ ID NO: 184, according to the Contact antibody numbering scheme.

### CD122 Binding Affinity

In some embodiments, anti-CD122 antibodies and antigen binding fragments thereof described herein bind with a measurable affinity to CD122. In some embodiments, the anti-CD122 antibody or antigen binding fragment thereof binds to mammalian CD122. In some embodiments, the anti-CD122 antibody or antigen binding fragment thereof binds to mouse CD122. In some embodiments, the anti-CD122 antibody or antigen binding fragment thereof binds to human CD122. In some embodiments, the anti-CD122 antibody or antigen binding fragment thereof that binds to CD122 comprises an HCDR1 comprising the amino acid sequence of SEQ ID NO: 1; an HCDR2 comprising the amino acid sequence of SEQ ID NO: 12; an HCDR3 comprising the amino acid sequence of SEQ ID NO: 24; an LCDR1 comprising the amino acid sequence of SEQ ID NO: 37; an LCDR2 comprising the amino acid sequence of GTS; and an LCDR3 comprising the amino acid sequence of SEQ ID NO: 56. In some embodiments, the anti-CD122 antibody or antigen binding fragment thereof that binds to CD122 comprises an HCDR1 comprising the amino acid sequence of SEQ ID NO: 2; an HCDR2 comprising the amino acid sequence of SEQ ID NO: 13; an HCDR3 comprising the amino acid sequence of SEQ ID NO: 25; an LCDR1 comprising the amino acid sequence of SEQ ID NO: 38; an LCDR2 comprising the amino acid sequence of TTS; and an LCDR3 comprising the amino acid sequence of SEQ ID NO: 57. In some embodiments, the anti-CD122 antibody or antigen binding fragment thereof that binds to CD122 comprises an HCDR1 comprising the amino acid sequence of SEQ ID NO: 3; an HCDR2 comprising the amino acid sequence of SEQ ID NO: 14; an HCDR3 comprising the amino acid sequence of SEQ ID NO: 26; an LCDR1 comprising the amino acid sequence of SEQ ID NO: 39; an LCDR2 comprising the amino acid sequence of YTS; and an LCDR3 comprising the amino acid sequence of SEQ ID NO: 58. In some embodiments, the anti-CD122 antibody or antigen binding fragment thereof that binds to CD122 comprises an HCDR1 comprising the amino acid sequence of SEQ ID NO: 4; an HCDR2 comprising the amino acid sequence of SEQ ID NO: 15; an HCDR3 comprising the amino acid sequence of SEQ ID NO: 27; an LCDR1 comprising the amino acid sequence of SEQ ID NO: 40; an LCDR2 comprising the amino acid sequence of WAS; and an LCDR3 comprising the amino acid sequence of SEQ ID NO: 59. In some embodiments, the anti-CD122 antibody or antigen binding fragment thereof that binds to CD122 comprises an HCDR1 comprising the amino acid sequence of SEQ ID NO: 5; an HCDR2 comprising the amino acid sequence of SEQ ID NO: 16; an HCDR3 comprising the amino acid sequence of SEQ ID NO: 28; an LCDR1 comprising the amino acid sequence of SEQ ID NO: 41; an LCDR2 comprising the amino acid sequence of YTS; and an LCDR3 comprising the amino acid sequence of SEQ ID NO: 60. In some embodiments, the anti-CD122 antibody or antigen binding fragment thereof that binds to CD122 comprises an HCDR1 comprising the amino acid sequence of SEQ ID NO: 6; an HCDR2 comprising the amino acid sequence of SEQ ID NO: 17; an HCDR3 comprising the amino acid sequence of SEQ ID NO: 29; an LCDR1 comprising the amino acid sequence of SEQ ID NO: 42; an LCDR2 comprising the amino acid sequence of KAS; and an LCDR3 comprising the amino acid sequence of SEQ ID NO: 61. In some embodiments, the anti-CD122 antibody or antigen binding fragment thereof that binds to CD122 comprises an HCDR1 comprising the amino acid sequence of SEQ ID NO: 7; an HCDR2 comprising the amino acid sequence of SEQ ID NO: 18; an HCDR3 comprising the amino acid sequence of SEQ ID NO: 30; an LCDR1 comprising the amino acid sequence of SEQ ID NO: 43; an LCDR2 comprising the amino acid sequence of YTS; and an LCDR3 comprising the amino acid sequence of SEQ ID NO: 62. In some embodiments, the anti-CD122 antibody or antigen binding fragment thereof that binds to CD122 comprises an HCDR1 comprising the amino acid sequence of SEQ ID NO: 8; an HCDR2 comprising the amino acid sequence of SEQ ID NO: 19; an HCDR3 comprising the amino acid sequence of SEQ ID NO: 31; an LCDR1 comprising the amino acid sequence of SEQ ID NO: 44; an LCDR2 comprising the amino acid sequence of YTS; and an LCDR3 comprising the amino acid sequence of SEQ ID NO: 63. In some embodiments, the anti-CD122 antibody or antigen binding fragment thereof that binds to CD122 comprises an HCDR1 comprising the amino acid sequence of SEQ ID NO: 9; an HCDR2 comprising the amino acid sequence of SEQ ID NO: 20; an HCDR3 comprising the amino acid sequence of SEQ ID NO: 32; an LCDR1 comprising the amino acid sequence of SEQ ID NO: 45; an LCDR2 comprising the amino acid sequence of GAT; and an LCDR3 comprising the amino acid sequence of SEQ ID NO: 64. In some embodiments, the anti-CD122 antibody or antigen binding fragment thereof that binds to CD122 comprises an HCDR1 comprising the amino acid sequence of SEQ ID NO: 1; an HCDR2 comprising the amino acid sequence of SEQ ID NO: 21; an HCDR3 comprising the amino acid sequence of SEQ ID NO: 33; an LCDR1 comprising the amino acid sequence of SEQ ID NO: 37; an LCDR2 comprising the amino acid sequence of GTS; and an LCDR3 comprising the amino acid sequence of SEQ ID NO: 65. In some embodiments, the anti-CD122 antibody or antigen binding fragment thereof that binds to CD122 comprises an HCDR1 comprising the amino acid sequence of SEQ ID NO: 1; an HCDR2 comprising the amino acid sequence of SEQ ID NO: 21; an HCDR3 comprising the amino acid sequence of SEQ ID NO: 34; an LCDR1 comprising the amino acid sequence of SEQ ID NO: 37; an LCDR2 comprising the amino acid sequence of GTS; and an LCDR3 comprising the amino acid sequence of SEQ ID NO: 65. In some embodiments, the anti-CD122 antibody or antigen binding fragment thereof that binds to CD122 comprises an HCDR1 comprising the amino acid sequence of SEQ ID NO: 10; an HCDR2 comprising the amino acid sequence of SEQ ID NO: 22; an HCDR3 comprising the amino acid sequence of SEQ ID NO: 35; an LCDR1 comprising the amino acid sequence of SEQ ID NO: 46; an LCDR2 comprising the amino acid sequence of YAS; and an LCDR3 comprising the amino acid sequence of SEQ ID NO: 66. In some embodiments, the anti-CD122 antibody or antigen binding fragment thereof that binds to CD122 comprises an HCDR1 comprising the amino acid sequence of SEQ ID NO: 11; an HCDR2 comprising the amino acid sequence of SEQ ID NO: 23; an HCDR3 comprising the amino acid sequence of SEQ ID NO: 36; an LCDR1 comprising the amino acid sequence of SEQ ID NO: 47; an LCDR2 comprising the amino acid sequence of STS; and an LCDR3 comprising the amino acid sequence of SEQ ID NO: 67. In some embodiments, the binding affinity of the antibody for CD122 is a binding affinity as measured by K_{D} (equilibrium dissociation constant).

In some embodiments, the anti-CD122 antibodies and antigen binding fragments thereof described herein bind with a measurable affinity to CD122 as measured by K_{D}. In some embodiments, the anti-CD122 antibody is a mouse antibody. In some embodiments, the anti-CD122 antibody is a humanized antibody. In some embodiments, the anti-CD122 antibody is a human antibody. In some embodiments, the antibody or antigen binding fragment thereof binds specifically to CD122. In some embodiments, the antibody or antigen binding fragment thereof exhibits moderate to low non-specific binding (NBS) to unintended peptides, proteins, receptors, or transporters. In some embodiments, the antibody or antigen binding fragment thereof exhibits low non-specific binding (NBS) to unintended peptides, proteins, receptors, or transporters. In some embodiments, the antibody or antigen binding fragment thereof binds specifically to a mammalian CD122. In some embodiments, the antibody or antigen binding fragment thereof binds to mouse CD122. In some embodiments, the antibody or antigen binding fragment thereof binds to human CD122. In some embodiments, the antibody or antigen binding fragment thereof binds to CD122 protein or a domain of CD122 protein with a binding affinity as measured by K_{D} of about from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁶ M to 10⁻¹⁰ M, from 10⁻⁷ M to 10⁻¹⁰ M, from 10⁻⁸ M to 10⁻¹⁰ M, from 10⁻⁹ M to 10⁻¹⁰ M, from 10⁻⁶ M to 10⁻⁹ M, from 10⁻⁷ M to 10⁻⁹ M, from 10⁻⁸ M to 10⁻⁹ M, from 10⁻⁶ M to 10⁻⁸ M, from 10⁻⁷ M to 10⁻⁸ M. or from 10⁻⁶ M to 10⁻⁷ M. In some embodiments, the antibody or antigen binding fragment thereof binds to one or more epitopes within the extracellular domain of CD122 protein, with a binding affinity as measured by K_{D} of about from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁶ M to 10⁻¹⁰ M, from 10⁻⁷ M to 10⁻¹⁰ M, from 10⁻⁸ M to 10⁻¹⁰ M, from 10⁻⁹ M to 10⁻¹⁰ M, from 10⁻⁶ M to 10⁻⁹ M, from 10⁻⁷ M to 10⁻⁹ M, from 10⁻⁸ M to 10⁻⁹ M, from 10⁻⁶ M to 10⁻⁸ M, from 10⁻⁷ M to 10⁻⁸ M. or from 10⁻⁶ M to 10⁻⁷ M. In some embodiments, the antibody or antigen binding fragment thereof binds to human CD122 protein with a binding affinity as measured by K_{D} of less than about 9E⁻⁰⁸ M, 8E⁻⁰⁸ M, 7E⁻⁰⁸ M, 6E⁻⁰⁸ M, 5E⁻⁰⁸ M, 4E08 M, 3E⁻⁰⁸ M, 2E⁻⁰⁸ M, 1E⁻⁰⁸ M, 9E⁻⁰⁹ M, 8E⁻⁰⁹ M, 7E⁻⁰⁹ M, 6E⁻⁰⁹ M, 5E⁻⁰⁹ M, 4E⁻⁰⁹ M, 3E⁻⁰⁹ M, 2E⁻⁰⁹ M, 1E⁻⁰⁹ M, 9E⁻¹⁰ M, 8E⁻¹⁰ M, 7E⁻¹⁰ M, 6E⁻¹⁰ M, 5E⁻¹⁰ M, 4E⁻¹⁰ M, 3E⁻¹⁰ M, 2E⁻¹⁰ M, or 1E⁻¹⁰ M, In some embodiments, the antibody binds to human CD122 protein with a binding affinity in the nanomolar range (K_{D} value of 10⁻⁷ to 10⁻⁹M). In some embodiments, the antibody binds to human CD122 protein with a binding affinity in the low nanomolar range (K_{D} value of 10⁻⁹ M). In some embodiments, the antibody binds to human CD122 protein with a binding affinity in the picomolar range (K_{D} value of 10⁻¹⁰ to 10⁻¹² M). In some embodiments, the antibody binds to human CD122 protein with a binding affinity in the high picomolar range (K_{D} value of 10⁻¹⁰ M). In some embodiments, the antibody binds to human CD122 protein with a binding affinity falling within the low nanomolar range to the high picomolar range (K_{D} value of 10⁻⁹ to 10⁻¹⁰ M). In some embodiments, the antibody binds to human CD122 protein with a binding affinity as measured by K_{D} of less than about 3E⁻⁰⁹ M. In some embodiments, the antibody binds to human CD122 protein with a binding affinity as measured by K_{D} of less than about 2E⁻⁰⁹ M. In some embodiments, the antibody binds to human CD122 protein with a binding affinity as measured by K_{D} of less than about 1E⁻⁰⁹ M. In some embodiments, the antibody binds to human CD122 protein with a binding affinity as measured by K_{D} of less than about 9E⁻¹⁰ M. In some embodiments, the antibody binds to human CD122 protein with a binding affinity as measured by K_{D} of less than about 8E⁻¹⁰ M. In some embodiments, the antibody binds to human CD122 protein with a binding affinity as measured by K_{D} of between about 5E⁻⁰⁹ to 5E⁻¹⁰ M. In some embodiments, the antibody binds to human CD122 protein with a binding affinity as measured by K_{D} of between about 3E⁻⁰⁹ to 7E⁻¹⁰ M. In some embodiments, the antibody binds to human CD122 protein with a binding affinity as measured by K_{D} of between about 2E⁻⁰⁹ to 8E⁻¹⁰ M. the antibody binds to human CD122 protein with a binding affinity as measured by K_{D} of between about 1E⁻⁰⁹ to 9E⁻¹⁰ M.

In some embodiments, the antibody or antigen binding fragment thereof is measured as binding to mouse CD122 protein by assaying antibody binding affinity to a mouse CD122 protein sequence listed in Table 15. In some embodiments, the antibody or antigen binding fragment thereof is measured as binding to mouse CD122 protein by assaying antibody binding affinity to a portion of a mouse CD122 protein sequence listed in Table 15. In some embodiments, the antibody or antigen binding fragment thereof is measured as binding to mouse CD122 protein by assaying antibody binding affinity to an extracellular portion of a mouse CD122 protein sequence listed in Table 15 containing the CD122 extracellular domain (A26-E240). In some embodiments, the antibody or antigen binding fragment thereof is measured as binding to human CD122 protein by assaying antibody binding affinity to a human CD122 protein sequence listed in Table 15. In some embodiments, the antibody or antigen binding fragment thereof is measured as binding to human CD122 protein by assaying antibody binding affinity to a portion of a human CD122 protein sequence listed in Table 15. In some embodiments, the antibody or antigen binding fragment thereof is measured as binding to human CD122 protein by assaying antibody binding affinity a polypeptide sequence comprising a portion of an amino acid sequence listed in Table 15. In some embodiments, the antibody or antigen binding fragment thereof is measured as binding to human CD122 protein by assaying antibody binding affinity to an extracellular portion of a human CD122 protein sequence listed in Table 15 containing the CD122 extracellular domain (A26-D239). In some embodiments, the anti-CD122 antibody or antigen binding fragment thereof cross-competes for binding to CD122 with another anti-CD122 antibody or antigen-binding fragment thereof described herein. In some embodiments, the anti-CD122 antibody or antigen binding fragment thereof cross-competes for binding to CD122 with a known anti-CD122 antibody.

TABLE 15
Mouse and Human CD122 protein sequences

| SEQ | | |
|---|---|---|
| ID | | |
| NO: | Description | Protein Sequence |
| 185 | Mouse CD122 | MATIALPWSLSLYVFLLLLATPWASAAVKNCSHLECFYNSRANVSCMWS |
| | precursor | HEEALNVTTCHVHAKSNLRHWNKTCELTLVRQASWACNLILGSFPESQS |
| | protein | LTSVDLLDINVVCWEEKGWRRVKTCDFHPEDNLRLVAPHSLQVLHIDTQ |
| | sequence | RCNISWKVSQVSHYIEPYLEFEARRRLLGHSWEDASVLSLKQRQQWLEL |
| | | EMLIPSTSYEVQVRVKAQRNNTGTWSPWSQPLTFRTRPADPMKEILPMS |
| | | WLRYLLLVLGCFSGFFSCVYILVKCRYLGPWLKTVLKCHIPDPSEFFSQ |
| | | LSSQHGGDLQKWLSSPVPLSFFSPSGPAPEISPLEVLDGDSKAVQLLLL |
| | | QKDSAPLPSPSGHSQASCFTNQGYFFFHLPNALEIESCQVYFTYDPCVE |
| | | EEVEEDGSRLPEGSPHPPLLPLAGEQDDYCAFPPRDDLLLESPSLSTPN |
| | | TAYGGSRAPEERSPLSLHEGLPSLASRDLMGLQRPLERMPEGDGEGLSA |
| | | NSSGEQASVPEGNLHGQDQDRGQGPILTLNTDAYLSLQELQAQDSVHLI |
| 186 | Human CD122 | MAAPALSWRLPLLILLLPLATSWASAAVNGTSQFTCFYNSRANISCVWS |
| | precursor | QDGALQDTSCQVHAWPDRRRWNQTCELLPVSQASWACNLILGAPDSQKL |
| | protein | TTVDIVTLRVLCREGVRWRVMAIQDFKPFENLRLMAPISLQVVHVETHR |
| | sequence | CNISWEISQASHYFERHLEFEARTLSPGHTWEEAPLLTLKQKQEWICLE |
| | | TLTPDTQYEFQVRVKPLQGEFTTWSPWSQPLAFRTKPAALGKDTIPWLG |
| | | HLLVGLSGAFGFIILVYLLINCRNTGPWLKKVLKCNTPDPSKFFSQLSS |
| | | EHGGDVQKWLSSPFPSSSFSPGGLAPEISPLEVLERDKVTQLLLQQDKV |
| | | PEPASLSSNHSLTSCFTNQGYFFFHLPDALEIEACQVYFTYDPYSEEDP |
| | | DEGVAGAPTGSSPQPLQPLSGEDDAYCTFPSRDDLLLESPSLLGGPSPP |
| | | STAPGGSGAGEERMPPSLQERVPRDWDPQPLGPPTPGVPDLVDFQPPPE |
| | | LVLREAGEEVPDAGPREGVSFPWSRPPGQGEFRALNARLPLNTDAYLSL |
| | | QELQGQDPTHLV |
| 187 | Human CD122 | AAVNGTSQFTCFYNSRANISCVWSQDGALQDTSCQVHAWPDRRRWNQTC |
| | extracellular | ELLPVSQASWACNLILGAPDSQKLTTVDIVTLRVLCREGVRWRVMAIQD |
| | domain (A26- | FKPFENLRLMAPISLQVVHVETHRCNISWEISQASHYFERHLEFEARTL |
| | D239) | SPGHTWEEAPLLTLKQKQEWICLETLTPDTQYEFQVRVKPLQGEFTTWS |
| | | PWSQPLAFRTKPAALGKD |
| 188 | Human CD122 | AVNGTSQFTCFYNSRANISCVWSQDGALQDTSCQVHAWPDRRRWNQTCE |
| | protein | LLPVSQASWACNLILGAPDSQKLTTVDIVTLRVLCREGVRWRVMAIQDE |
| | sequence | KPFENLRLMAPISLQVVHVETHRCNISWEISQASHYFERHLEFEARTLS |
| | | PGHTWEEAPLLTLKQKQEWICLETLTPDTQYEFQVRVKPLQGEFTTWSP |
| | | WSQPLAFRTKPAALGKDTIPWLGHLLVGLSGAFGFIILVYLLINCRNTG |
| | | PWLKKVLKCNTPDPSKFFSQLSSEHGGDVQKWLSSPFPSSSFSPGGLAP |
| | | EISPLEVLERDKVTQLLLQQDKVPEPASLSSNHSLTSCFTNQGYFFFHL |
| | | PDALEIEACQVYFTYDPYSEEDPDEGVAGAPTGSSPQPLQPLSGEDDAY |
| | | CTFPSRDDLLLESPSLLGGPSPPSTAPGGSGAGEERMPPSLQERVPRDW |
| | | DPQPLGPPTPGVPDLVDFQPPPELVLREAGEEVPDAGPREGVSFPWSRP |
| | | PGQGEFRALNARLPLNTDAYLSLQELQGQDPTHLV |

### Comparison to Known Anti-CD122 Antibodies

CD122 antibodies described herein may be compared by their properties, structures, and/or functional characteristics to other known anti-CD122 antibodies. In some embodiments, a humanized monoclonal antibody described herein has one or more CD122 binding epitopes determined. In some embodiments, the CD122 binding epitope comprises at least 1, 2, 3, 4, 5, 6, 7, 8, 9, 10, 11, 12, 13, 14, 15, 16, 17, 18, 19, 20, 21, or 22 amino acid residues of CD122 which are included in the CD122 binding epitope of the humanized monoclonal antibody. In some embodiments, the CD122 binding epitope comprises at least 1, 2, 3, 4, 5, 6, 7, 8, 9, 10, 11, 12, 13, 14, 15, 16, 17, 18, 19, 20, 21, or 22 amino acid residues of an extracellular portion of human CD122 which are included in the CD122 binding epitope of the humanized monoclonal antibody. In some embodiments, the amino acid residues of human CD122 comprising the anti-CD122 antibody epitope are distinct from the amino acid residues of human CD122 comprising an anti-CD122 antibody epitope of a known anti-CD122 antibody. In some embodiments, a humanized monoclonal anti-CD122 antibody described herein binds to a human CD122 epitope distinct from that to which other known anti-CD122 antibodies bind. In some embodiments, the human CD122 epitope to which a humanized monoclonal anti-CD122 antibody described herein binds is compared to human CD122 epitope to which a known humanized monoclonal anti-CD122 antibody binds. In some embodiments, a humanized monoclonal antibody described herein serves as more potent functional inhibitor of IL2-induced cellular signaling compared to one or more known anti-CD122 antibodies. In some embodiments, a humanized monoclonal antibody described herein serves as more potent functional inhibitor of IL15-induced cellular signaling compared to one or more known anti-CD122 antibodies. In some embodiments, a humanized monoclonal antibody described herein serves as more potent functional inhibitor of IL2-induced and IL-15 cellular signaling compared to one or more known anti-CD122 antibodies. In some embodiments, the properties, structures, and/or functional characteristics of a humanized anti-CD122 antibody described herein are compared to those of a known humanized anti-CD122 antibody comprising CDR sequences listed in Table 16. In some embodiments, the known humanized anti-CD122 antibody used to compare CD122 epitopes or functional properties such as an ability to serve as functional inhibitor or IL2-signaling, IL15-signaling, or IL-2 signaling and IL-15 signaling comprises a VH domain and a VL domain, wherein the VH domain comprises an HCDR1 comprising the sequence of SEQ ID NO: 189, an HCDR2 comprising the sequence of SEQ ID NO: 190, and an HCDR3 comprising the sequence of SEQ ID NO: 191; and wherein the VL domain comprises an LCDR1 comprising the sequence of SEQ ID NO: 192, an LCDR2 comprising the sequence of SEQ ID NO: 193, and an LCDR3 comprising the sequence of SEQ ID NO: 194.

TABLE 16
CDR sequences of known anti-CD122
antibodies

| SEQ | Anti-CD122 | |
|---|---|---|
| ID | Antibody | |
| NO: | CDRs | Amino Acid Sequence |
| 189 | HCDR1 | DFYME |
| 190 | HCDR2 | ASRNKANDYTTEYSASVKG |
| 191 | HCDR3 | SYYRYDGMDY |
| 192 | LCDR1 | SAISSVSYMY |
| 193 | LCDR2 | DTSNLVS |
| 194 | LCDR3 | QQWNTYPYT |

In some aspects, an isolated anti-CD122 antibody described herein, has one or more binding epitopes to a CD122 protein characterized. In some embodiments, the CD122 epitope to which an anti-CD122 antibody described herein binds is a functional epitope. In some embodiments, the CD122 epitope to which an anti-CD122 antibody described herein binds is a structural epitope. In some embodiments, the CD122 epitope to which an anti-CD122 antibody described herein binds is conformational rather than linear. In some embodiments, the conformational epitope comprises at least two, at least three, or at least four non-linear portions of CD122. In some embodiments, the epitope is an epitope on a native CD122 protein. In some embodiments, an isolated anti-CD122 antibody described herein inhibits binding of IL2 to a high affinity IL-αβγ receptor comprising CD122, CD132, and CD25. In some embodiments, an isolated anti-CD122 antibody described herein inhibits binding of IL15, presented in trans bound to IL15Rα, to the intermediate affinity IL-βγ receptor comprising CD122 and CD132. In some embodiments, an isolated anti-CD122 antibody described herein inhibits binding of IL15 to a high affinity IL-αβγ receptor comprising CD122, CD132 and IL15Ru. In some embodiments, an isolated anti-CD122 monoclonal antibody described herein binds to at least 1, 2, 3, 4, 5, 6, 7, 8, 9, 10, 11, 12, 13, 14, 15, 16, 17, 18, 19, 20, 21, or all 22 of the residues W39, P40, D41, V76, D77, 178, V79, T80, L81, F99, K100, P101, F102, E103, N104, L105, H134, Y135, F136, E137, R138, or H139 of the extracellular domain of a human CD122 protein according to amino acid numbering listed in SEQ ID NO: 187. In some embodiments, at least 1, 2, 3, 4, 5, 6, 7, 8, 9, 10, 11, 12, 13, 14, 15, 16, 17, 18, 19, 20, 21, or all 22 of the residues W39, P40, D41, V76, D77, 178, V79, T80, L81, F99, K100, P101, F102, E103, N104, L105, H134, Y135, F136, E137, R138, or H139 of the extracellular domain of a human CD122 protein according to amino acid numbering listed in SEQ ID NO: 187 comprise an epitope to which an isolated anti-CD122 monoclonal antibody described herein binds. In some embodiments, an isolated anti-CD122 monoclonal antibody described herein binds to at least one amino acid residue from epitope site 1) residues 39-41 (WPD) of the extracellular domain of a human CD122 protein comprising the amino acid sequence of SEQ ID NO: 187. In some embodiments, an isolated anti-CD122 monoclonal antibody described herein binds to at least one amino acid residue from epitope site 2) residues 76-81 (VDIVTL (SEQ ID NO: 195)) of the extracellular domain of a human CD122 protein comprising the amino acid sequence of SEQ ID NO: 187. In some embodiments, an isolated anti-CD122 monoclonal antibody described herein binds to at least one amino acid residue from epitope site 3) residues 99-105 (FKPFENL (SEQ ID NO: 196)) of the extracellular domain of a human CD122 protein comprising the amino acid sequence of SEQ ID NO: 187. In some embodiments, an isolated anti-CD122 monoclonal antibody described herein binds to at least one amino acid residue from epitope site 4) residues 134-139 (HYFERH (SEQ ID NO: 197)) of the extracellular domain of a human CD122 protein comprising the amino acid sequence of SEQ ID NO: 187. In some embodiments, an isolated anti-CD122 monoclonal antibody described herein binds to at least one, two, or three of amino acid residues from epitope site 1) residues 39-41 (WPD) of the extracellular domain of a human CD122 protein comprising the amino acid sequence of SEQ ID NO: 187. In some embodiments, an isolated anti-CD122 monoclonal antibody described herein binds to at least one, two, three, four, five, or six of amino acid residues from epitope site 2) residues 76-81 (VDIVTL (SEQ ID NO: 195)) of the extracellular domain of a human CD122 protein comprising the amino acid sequence of SEQ ID NO: 187. In some embodiments, an isolated anti-CD122 monoclonal antibody described herein binds to at least one, two, three, four, five, six, or seven of amino acid residues from epitope site 3) residues 99-105 (FKPFENL (SEQ ID NO: 196)) of the extracellular domain of a human CD122 protein comprising the amino acid sequence of SEQ ID NO: 187. In some embodiments, an isolated anti-CD122 monoclonal antibody described herein binds to at least one, two, three, four, five, or six of amino acid residues from epitope site 4) residues 134-139 (HYFERH (SEQ ID NO: 197)) of the extracellular domain of a human CD122 protein comprising the amino acid sequence of SEQ ID NO: 187. In some embodiments, an isolated anti-CD122 monoclonal antibody described herein binds to at least one amino acid residue from epitope site 1) residues 39-41 (WPD) and: i) at least one amino acid residue from epitope site 2) residues 76-81 (VDIVTL (SEQ ID NO: 195)) of the extracellular domain of a human CD122 protein comprising the amino acid sequence of SEQ ID NO: 187; or ii) at least one amino acid residue from epitope site 3) residues 99-105 (FKPFENL (SEQ ID NO: 196)) of the extracellular domain of a human CD122 protein comprising the amino acid sequence of SEQ ID NO: 187; or iii) at least one amino acid residue from epitope site 4) residues 134-139 (HYFERH (SEQ ID NO: 197)) of the extracellular domain of a human CD122 protein comprising the amino acid sequence of SEQ ID NO: 187. In some embodiments, an isolated anti-CD122 monoclonal antibody described herein binds to at least one amino acid residue from epitope site 2) residues 76-81 (VDIVTL (SEQ ID NO: 195)) and: i) at least one amino acid residue from epitope site 3) residues 99-105 (FKPFENL (SEQ ID NO: 196)) of the extracellular domain of a human CD122 protein comprising the amino acid sequence of SEQ ID NO: 187; or ii) at least one amino acid residue from epitope site 4) residues 134-139 (HYFERH (SEQ ID NO: 197)) of the extracellular domain of a human CD122 protein comprising the amino acid sequence of SEQ ID NO: 187. In some embodiments, an isolated anti-CD122 monoclonal antibody described herein binds to at least one amino acid residue from epitope site 3) residues 99-105 (FKPFENL (SEQ ID NO: 196)) and at least one amino acid residue from epitope site 4) residues 134-139 (HYFERH (SEQ ID NO: 197)) of the extracellular domain of a human CD122 protein comprising the amino acid sequence of SEQ ID NO: 187. In some embodiments, an isolated anti-CD122 monoclonal antibody described herein binds to at least one, two, or three of amino acid residues from epitope site 1) residues 39-41 (WPD) and: i) at least one, two, three, four, five, or six of amino acid residues from epitope site 2) residues 76-81 (VDIVTL (SEQ ID NO: 195)) of the extracellular domain of a human CD122 protein comprising the amino acid sequence of SEQ ID NO: 187; or ii) at least one, two, three, four, five, six, or seven of amino acid residues from epitope site 3) residues 99-105 (FKPFENL (SEQ ID NO: 196)) of the extracellular domain of a human CD122 protein comprising the amino acid sequence of SEQ ID NO: 187; or iii) at least one, two, three, four, five, or six of amino acid residues from epitope site 4) residues 134-139 (HYFERH (SEQ ID NO: 197)) of the extracellular domain of a human CD122 protein comprising the amino acid sequence of SEQ ID NO: 187. In some embodiments, an isolated anti-CD122 monoclonal antibody described herein binds to at least one, two, three, four, five, or six of amino acid residues from epitope site 2) residues 76-81 (VDIVTL (SEQ ID NO: 193)) and: i) at least one, two, three, four, five, six, or seven of amino acid residues from epitope site 3) residues 99-105 (FKPFENL (SEQ ID NO: 194)) of the extracellular domain of a human CD122 protein comprising the amino acid sequence of SEQ ID NO: 187; or ii) at least one, two, three, four, five, or six of amino acid residues from epitope site 4) residues 134-139 (HYFERH (SEQ ID NO: 197)) of the extracellular domain of a human CD122 protein comprising the amino acid sequence of SEQ ID NO: 187. In some embodiments, an isolated anti-CD122 monoclonal antibody described herein binds to at least one, two, three, four, five, six, or seven of amino acid residues from epitope site 3) residues 99-105 (FKPFENL (SEQ ID NO: 196)) and at least one, two, three, four, five, or six of amino acid residues from epitope site 4) residues 134-139 (HYFERH (SEQ ID NO: 197)) of the extracellular domain of a human CD122 protein comprising the amino acid sequence of SEQ ID NO: 187.

### Production and Manufacture of Antibodies or Antigen Binding Fragments Thereof

In some embodiments, polypeptides described herein (e.g., antibodies or antigen binding fragments thereof) are produced using any method known in the art to be useful for the synthesis of polypeptides (e.g., antibodies), in particular, by chemical synthesis or by recombinant expression, and are preferably produced by use of a recombinant expression technique.

In some instances, an antibody or antigen binding fragment thereof is expressed recombinantly, and the nucleic acid encoding the antibody or its antigen binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 5′ and 3′ ends of the sequence or by cloning using an oligonucleotide specific for the particular nucleic acid sequence.

In some instances, an antibody or its binding is optionally made by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937.

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, a nucleic acid sequence encodes the antibodies disclosed herein. In some embodiments, the polynucleotide sequence encoding the antibodies is operatively coupled to a eukaryotic regulatory sequence. In some embodiments, a cell comprises the nucleic acid sequence. In some embodiments, a cell comprises a nucleic acid encoding the antibodies disclosed herein. In some embodiments, the cell comprises a prokaryotic cell. In some embodiments, the prokaryotic cell is an Escherichia coli cell. In some embodiments, the cell comprises a eukaryotic cell. In some embodiments, the eukaryotic cell is a Chines Hamster Ovary (CHO) cell, a HEK293 cell, a BHK cell, an NS0 murine myeloma cell, or a PER.C6® human cell. In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific aspects, the expression of the antibody is regulated by a constitutive, an inducible or a tissue, specific promoter. Standard cell lines and methods for the production of antibodies from a large-scale cell culture are known in the art. See e.g., Li et al., “Cell culture processes for monoclonal antibody production.” Mabs. 2010 September-October; 2(5): 466-477.

In certain aspects, described herein is a method of making antibodies comprising culturing a cell comprising a nucleic acid encoding antibodies under conditions in vitro sufficient to allow production and secretion of the antibodies. In some embodiments, antibodies are harvested from the cell culture medium. The harvesting can further comprise one or more purification steps to remove live cells, cellular debris, non-antibody proteins or polypeptides, undesired salts, buffers, and medium components. In certain aspects, the additional purification step(s) include centrifugation, ultracentrifugation, protein A, protein G, protein A/G, or protein L purification, and/or ion exchange chromatography.

### Pharmaceutical Compositions

Provided here are pharmaceutical compositions comprising an antibody or an antibody fragment and at least one pharmaceutically acceptable carrier. In some embodiments, the antibody or antibody fragment binds to CD122. In some embodiments, the antibody or antibody fragment is an anti-CD122 antibody or anti-CD122 antibody fragment. In some embodiments, the pharmaceutical compositions comprises an anti-CD122 antibody described herein. In some embodiments, the antibody or antibody fragment binds to human CD122. In some embodiments, the antibody or an antibody fragment and at least one pharmaceutically acceptable carrier are formulated into a pharmaceutical formulation. In some embodiments, the pharmaceutical formulation is chosen based on a preferred route of administration of the antibody or antibody fragment to a subject.

In some embodiments, the pharmaceutical formulations include, but are not limited to, aqueous liquid dispersions, self-emulsifying dispersions, solid solutions, liposomal dispersions, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanorods, nanoropes, and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle. In some instances, nanoparticles comprise nanospheres or nanocapsules.

In some instances, a nanoparticle is further coated with molecules for attachment of functional elements (e.g., with one or more of a polynucleic acid molecule or binding moiety described herein). In some instances, a coating comprises chondroitin sulfate, dextran sulfate, carboxymethyl dextran, alginate, pectin, carragheenan, fucoidan, agaropectin, porphyran, karaya gum, xanthan gum, hyaluronic acids, glucosamine, galactosamine, chitosan, polyglutamic acid, polyaspartic acid, lysozyme, cytochrome C, trypsinogen, chymotrypsinogen, α-chymotrypsin, polylysine, polyarginine, histone, protamine, ovalbumin or dextrin or cyclodextrin.

In some embodiments, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular, intra-arterial, intraperitoneal, intrathecal, intracerebral, intracerebroventricular, or intracranial) administration. In other instances, the pharmaceutical composition describe herein is formulated for oral administration. In still other instances, the pharmaceutical composition describe herein is formulated for intranasal administration.

### 1. Pharmaceutically Acceptable Excipients, Carriers, and Diluents

Compositions comprising the antibodies of the current disclosure are included in a pharmaceutical composition comprising one or more pharmaceutically acceptable excipients, carriers, and diluents. In some embodiments, the antibodies of the current disclosure are administered suspended in a sterile solution. In some embodiments, the antibodies of the current disclosure are administered suspended in an isotonic solution. In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate. In certain aspects, the solution comprises about 0.9% NaCl. In certain aspects, the solution comprises about 5.0% dextrose. In certain aspects, the solution further comprises one or more of buffers, for example, acetate, citrate, histidine, succinate, phosphate, bicarbonate and Tris(hydroxymethyl)aminomethane; surfactants, for example, polysorbate 80 (Tween 80), polysorbate 20 (Tween 20); polyol/disaccharide/polysaccharides, for example, glucose, dextrose, mannose, mannitol, sorbitol, sucrose, and dextran 40; amino acids, for example, glycine or arginine; antioxidants, for example, ascorbic acid, methionine; or chelating agents, for example, EDTA or EGTA. Carbomers in an aqueous pharmaceutical composition serve as emulsifying agents and viscosity modifying agents. In certain aspects, the pharmaceutically acceptable excipient comprises or consists of a carbomer. In certain aspects, the carbomer comprises or consists of carbomer 910, carbomer 934, carbomer 934P, carbomer 940, carbomer 941, carbomer 1342, or combinations thereof. Cyclodextrins in an aqueous pharmaceutical composition serve as solubilizing and stabilizing agents. In certain aspects, the pharmaceutically acceptable excipient comprises or consists of a cyclodextrin. In certain aspects, the cyclodextrin comprises or consists of alpha cyclodextrin, beta cyclodextrin, gamma cyclodextrin, or combinations thereof. Lecithin in a pharmaceutical composition may serve as a solubilizing agent. In certain aspects, the solubilizing agent comprises or consists of lecithin. Poloxamers in a pharmaceutical composition serve as emulsifying agents, solubilizing agents, and dispersing agents. In certain aspects, the pharmaceutically acceptable excipient comprises or consists of a poloxamer. In certain aspects, the poloxamer comprises or consists of poloxamer 124, poloxamer 188, poloxamer 237, poloxamer 338, poloxamer 407, or combinations thereof. Polyoxyethylene sorbitan fatty acid esters in a pharmaceutical composition serve as emulsifying agents, solubilizing agents, surfactants, and dispersing agents. In certain aspects, the pharmaceutically acceptable excipient comprises or consists of a polyoxyethylene sorbitan fatty acid ester. In certain aspects, the polyoxyethylene sorbitan fatty acid ester comprises or consists of polysorbate 20, polysorbate 21, polysorbate 40, polysorbate 60, polysorbate 61, polysorbate 65, polysorbate 80, polysorbate 81, polysorbate 85, polysorbate 120, or combinations thereof. Polyoxyethylene stearates in a pharmaceutical composition serve as emulsifying agents, solubilizing agents, surfactants, and dispersing agents. In certain aspects, the pharmaceutically acceptable excipient comprises or consists of a polyoxyethylene stearate. In certain aspects, the polyoxyethylene stearate comprises or consists of polyoxyl 2 stearate, polyoxyl 4 stearate, polyoxyl 6 stearate, polyoxyl 8 stearate, polyoxyl 12 stearate, polyoxyl 20 stearate, polyoxyl 30 stearate, polyoxyl 40 stearate, polyoxyl 50 stearate, polyoxyl 100 stearate, polyoxyl 150 stearate, polyoxyl 4 distearate, polyoxyl 8 distearate, polyoxyl 12 distearate, polyoxyl 32 distearate, polyoxyl 150 distearate, or combinations thereof. Sorbitan esters in a pharmaceutical composition serve as emulsifying agents, solubilizing agents, and non-ionic surfactants, and dispersing agents. In certain aspects, the pharmaceutically acceptable excipient comprises or consists of a sorbitan ester. In certain aspects, the sorbitan ester comprises or consists of sorbitan laurate, sorbitan oleate, sorbitan palmitate, sorbitan stearate, sorbitan trioleate, sorbitan sesquioleate, or combinations thereof.

In certain aspects, the antibodies of the current disclosure are shipped and/or stored lyophilized and can then be reconstituted before administration. In certain aspects, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than about 7.5. In certain aspects, the pH can be between 4.5 and 7.5, 4.5 and 7.0, 4.5 and 6.5, 4.5 and 6.0, or 5.5 or 5.0

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “antibody” herein is used in the broadest sense and includes monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region. An antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. A monoclonal antibody can comprise a human IgG1 constant region or a human IgG4 constant region.

The terms “complementarity determining region,” and “CDR,” which are synonymous with “hypervariable region” or “HVR,” are known in the art and refer to non-contiguous sequences of amino acids within antibody variable regions, which confer antigen specificity and/or binding affinity. In general, there are three CDRs in each heavy chain variable region (CDR-H1, CDR-H2, CDR-H3; also referred to as HCDR1, HCDR2, HCDR3) and three CDRs in each light chain variable region (CDR-L1, CDR-L2, CDR-L3; also referred to as LCDR1, LCDR2, LCDR3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain aspects, the CDRs of the antibodies described herein can be defined by a method selected from Kabat, Chothia, IMGT, Aho, AbM, Contact, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (See e.g., Portolano et al., J. Immunol. 150:880-887 (1993); Clarkson et al., Nature 352:624-628 (1991)).

Among the provided antibodies are antibody fragments. An “antibody fragment” can refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; and single-chain antibody molecules (e.g., scFv or sFv). In particular aspects, the antibodies are single-chain antibody fragments comprising a variable heavy chain region and/or a variable light chain region, such as scFvs. Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., polypeptide linkers, and/or those that are not produced by enzyme digestion of a naturally occurring intact antibody.

A “binding moiety” refers to a portion of a molecule, peptide, polypeptide, antibody, or antibody fragment that mediates specific binding to a recited target or antigen or epitope. By way of example, the binding moiety of an antibody may comprise a heavy-chain/light-chain variable region pair or one or more complementarity determining regions (CDRs).

A “target” as referred to herein refers to the portion of a molecule that participates with a binding moiety of a molecule, peptide, polypeptide, antibody, or antibody fragment. A target can comprise an amino acid sequence and/or a carbohydrate, lipid or other chemical entity. An “antigen” is a target comprising a portion that is able to be bound by an adaptive immune molecule such as an antibody or antibody fragment, B-cell receptor, or T-cell receptor.

An “epitope” as described herein refers to the one or more contact regions of an antibody. The contact region of an antibody consists of a discreet number of amino acids contacted by amino acid residues of the antibody (generally CDR residues) and adjacent residues contiguous with the contact residues. For example, the contact region may consist of a continuous stretch of a target protein that is between 5 to 20 amino acids, 5 to 15 amino acids, or 5 to 10 amino acids. An antibody may bind more than one contact region that are separated by 10, 20, 30, 40, 50, 75, or 100 amino acids or more as a result of protein folding. Epitopes may be determined using X-ray crystallography, hydrogen-deuterium exchange mass-spec, alanine spanning mutagenesis, competition with excess synthetic peptides as determined by immunoblot, ELISA, surface plasmon radiance, flow cytometry or any other suitable protein binding assay.

The term “cross-compete” or “cross-competes” are used interchangeably herein to refer to the ability of an antibody or an antigen binding fragment thereof to interfere with the binding directly or indirectly through allosteric modulation of the anti-CD122 antibodies of the disclosure to the target CD122 (e.g., human CD122, an extracellular domain of human CD122, mouse CD122, or an extracellular domain of mouse CD122). The extent to which an antibody or antigen binding fragment thereof is able to interfere with the binding of another to the target, and therefore whether it can be said to cross-compete, may be determined using one or more competition binding assay. One example of a competition binding assay is Homogeneous Time Resolved Fluorescence (HTRF). Another example of a competition binding assay is an epitope binning analysis conducted using surface plasmon resonance (SPR).

A “humanized” antibody is an antibody in which all or substantially all CDR amino acid residues are derived from non-human CDRs and all or substantially all FR amino acid residues are derived from human FRs. A humanized antibody optionally can include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

“ADCC” or “antibody dependent cell-mediated cytotoxicity” as used herein, refers to the cell-mediated reaction wherein nonspecific cytotoxic cells that express FcγRs recognize bound antibody on a target cell and subsequently cause lysis of the target cell. ADCC can be correlated with binding to FcγRIIIa wherein increased binding to FcγRIIIa leads to an increase in ADCC activity. “ADCP” or antibody dependent cell-mediated phagocytosis, as used herein, can refer to the cell-mediated reaction wherein nonspecific cytotoxic cells that express FcγRs recognize bound antibody on a target cell and subsequently cause phagocytosis of the target cell.

The terms “polypeptide” and “protein” are used interchangeably and refers to a polymer of amino acid residues, and are not limited to a minimum length. Polypeptides, including the provided antibodies and antibody chains and other peptides, e.g., linkers and binding peptides, can include amino acid residues including natural and/or non-natural amino acid residues. The terms also include post-expression modifications of the polypeptide, for example, glycosylation, sialylation, acetylation, phosphorylation, and the like. In some embodiments, the polypeptides can contain modifications with respect to a native or natural sequence, as long as the protein maintains the desired activity. These modifications can be deliberate, as through site-directed mutagenesis, or can be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Amino acid sequence variants of the antibodies provided herein can be contemplated and conceived. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody amino acid sequence variants of an antibody can be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Antibody variants having one or more amino acid substitutions can be provided. Sites of interest for mutagenesis by substitution include the CDRs and FRs. Amino acid substitutions can be introduced into an antibody of interest and the products screened for a desired activity, e.g., retained/improved antigen binding, decreased immunogenicity, or improved ADCC or CDC.

Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, seine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine). Exemplary conservative amino acid substitutions are listed in Table 17.

TABLE 17
Exemplary Amino Acid Modifications

| Side Chain | Members |
|---|---|
| Hydrophobic | Met, Ala, Val, Leu Ile |
| Neutral Hydrophilic | Cys, Ser, Thr |
| Acidic | Asp, Glu |
| Basic | Asn, Gln, His, Lys, Arg |
| Residues that influence | Gly, Pro |
| chain orientation | |
| Aromatic | Trp, Tyr, Phe |

The antibodies described herein can be encoded by a nucleic acid. A nucleic acid is a type of polynucleotide comprising two or more nucleotide bases. In certain aspects, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large-scale cell culture are known in the art. See e.g., Li et al., “Cell culture processes for monoclonal antibody production.” Mabs. 2010 September-October; 2(5): 466-477. In certain aspects, the cell is a Eukaryotic cell. In certain aspects, the Eukaryotic cell is a mammalian cell. In certain aspects, the mammalian cell is a cell line useful for producing antibodies is a Chines Hamster Ovary cell (CHO) cell, an NS0 murine myeloma cell, or a PER.C6® cell. In certain aspects, the nucleic acid encoding the antibody is integrated into a genomic locus of a cell useful for producing antibodies. In certain aspects, described herein is a method of making an antibody comprising culturing a cell comprising a nucleic acid encoding an antibody under conditions in vitro sufficient to allow production and secretion of said antibody.

The section headings used herein are for organizational purposes only and are not to be construed as limiting the subject matter described.

### EXAMPLES

The following examples are included for illustrative purposes only and are not intended to limit the scope of the invention.

### Example 1: Humanization of Mouse Anti-CD122 Antibody

To facilitate the identification of anti-CD122 antibodies that could be used in human subjects, a mouse monoclonal anti-CD122 antibody was used as a starting point for antibody sequence analysis and homology modeling of antibody three-dimensional (3D) structure. This process was used to design humanized forms of anti-CD122 antibodies, produce the design antibodies, and then test functional attributes of the antibodies. This process of design and construction of humanized VH and VL genes is described in (Tsurishita et al. 2005; Methods 36:69-83).

The VH and VL regions of a mouse monoclonal anti-CD122 antibody were substituted into both a human IgG1 and a human IgG4 backbone to create parental chimeric sequences. Sequence analysis and homology modeling of 3D antibody structure was undertaken to identify key positions supporting CDR loop structure and VH-VL interface. These results were used in determining specific amino acid substitutions to select in the design of humanized variants of the VH and VL regions. Three VH variants and three VL variants were selected based on the modeling results and proceeded with further testing in both an IgG1 backbone and an IgG4 backbone. Each HC-LC pairing was reformatted in two ways: with a human IgG1 (G1M17 allotype) and a human IgG4 (S228P isotype) backbone. The parental mouse various regions were reformatted as a hIgG1 or hIgG4 chimera similarly. A gene sequence encoding each designed VH and VL region was constructed for subsequent cloning into a mammalian expression vector.

An assessment of humanness of each pairing of one of the VH variants and one of the VL variants in both an IgG1 backbone and an IgG4 backbone was undertaken using a T20 humanness score (Gao et al. 2013, BMC Biotechnol. 13:55). A sufficiently high T20 score indicates a likelihood of eliminating immunogenicity issues to the same extent as using fully human antibodies in a human subject.

The humanness scores for the parental and humanized antibodies are shown in the Table 18 (heavy chain) and Table 19 (kappa light chain). Based on this method, a score of 84 or above is indicative of a human-like heavy chain framework, and a score of 90 or above is indicative of humanness for a kappa light chain framework. For full-length variable regions, cutoffs of 79 for the VH and 86 for the VL are recommended. In some embodiments, variable light chain regions are referred to as VK regions.

TABLE 18
Humanness assessment for selected VH domain sequences

| | | | T20 | T20 |
|---|---|---|---|---|
| | | | Analyzer | Analyzer |
| | | | Score | Score |
| HCDRs from | | | (Full | (Framework |
| CDR Series | Species | Framework | length) | only) |
| E7 | Mus Musculus | F7 | 67.9 | 72.0 |
| E7 | Homo sapiens | F4 | 81.5 | 92.3 |
| E7 | Homo sapiens | F5 | 83.2 | 90.4 |
| E7 | Homo sapiens | F6 | 84.1 | 92.8 |

TABLE 19
Humanness assessment for selected VL domain sequences

| | | | T20 | T20 |
|---|---|---|---|---|
| | | | Analyzer | Analyzer |
| | | | Score | Score |
| LCDRs from | | | (Full | (Framework |
| CDR Series | Species | Framework | length) | only) |
| E7 | Mus Musculus | F14 | 74.4 | 79.5 |
| E7 | Homo sapiens | F11 | 84.4 | 92.9 |
| E7 | Homo sapiens | F12 | 88.5 | 95.2 |
| E7 | Homo sapiens | F13 | 88.5 | 99.0 |

### Results:

T20 Analyzer score of humanness ranged from 81-88 for the humanized full-length variable regions (VH and VK) and ranged from 90-99 for the humanized variable region frameworks, which exceeded the threshold of humanness. Hu5-LC1 full length sequence was below the recommended cut off score of 86.

A VK sequence liability was identified. Asp, in DGTLK (parental sequence) (SEQ ID NO: 198), has potential to undergo isomerization in the framework region. This liability was removed in humanized variants.

### Production:

Two Parental chimeric and 18 Humanized Fab variants were transiently produced using a 0.01 L in CHO cells (TunaCHO™ extended 14-day process) and purified by protein A affinity-based purification. Endotoxin levels were assayed and 7 of the 18 Humanized Fab variants were also produced in a second production batch. As production yields may be important for the use of these antibodies, the second production batch was undertaken of some of the antibodies in order to compare yields.

### Binding Affinity of Chimeric and Humanized Antibodies:

19 antibodies were assayed for binding to human CD122 (huCD122). Their affinities (KD) values are reported in the summary table below and in subsequent slides alongside sensorgrams. The humanized variants from both batches showed less than 3-fold changes in KD values compared to the parental chimeras.

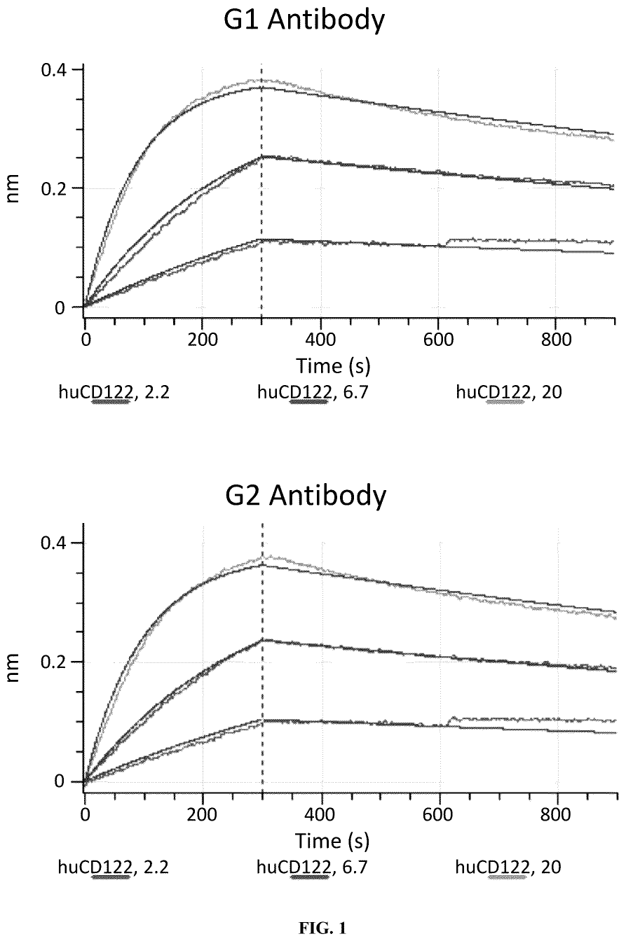

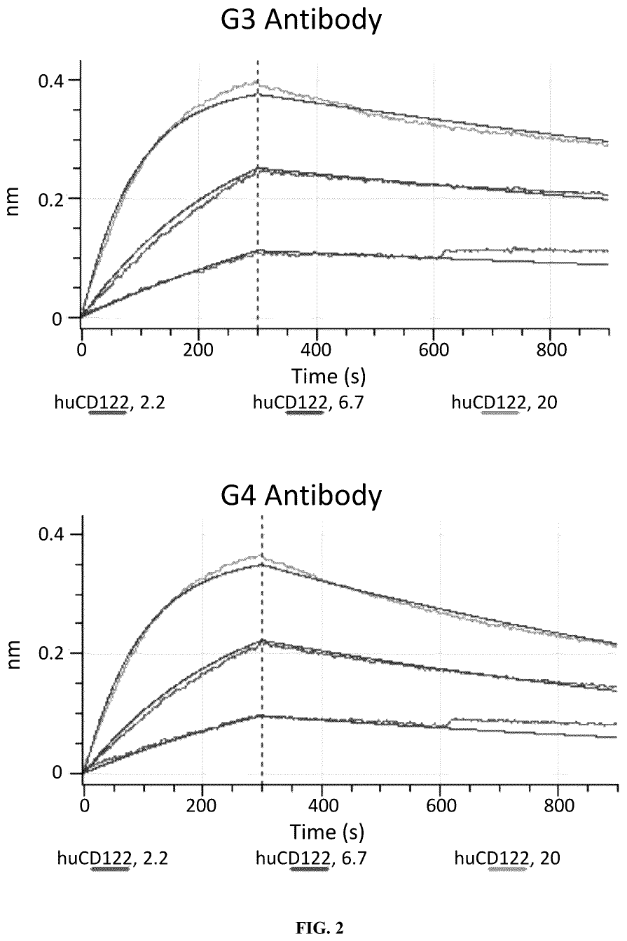

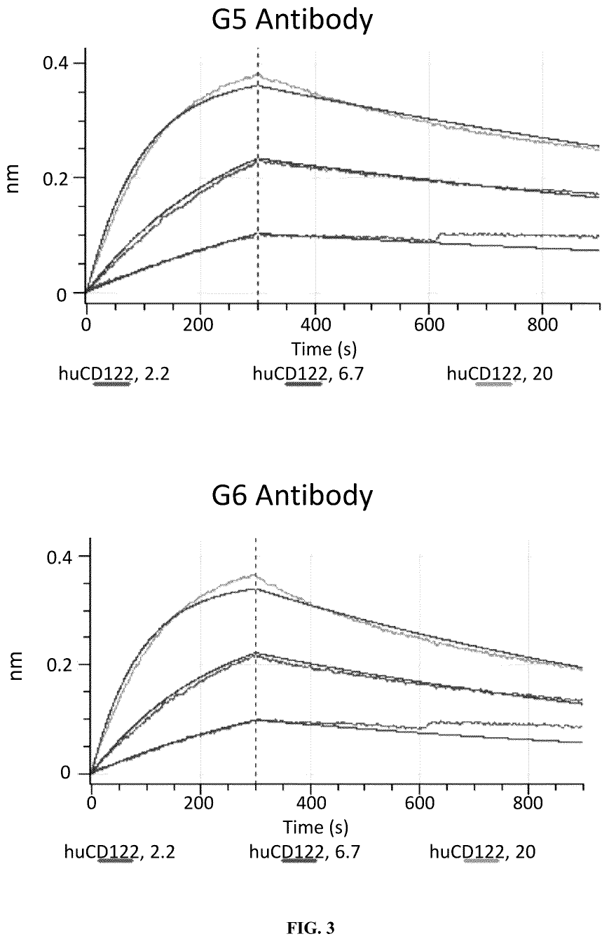

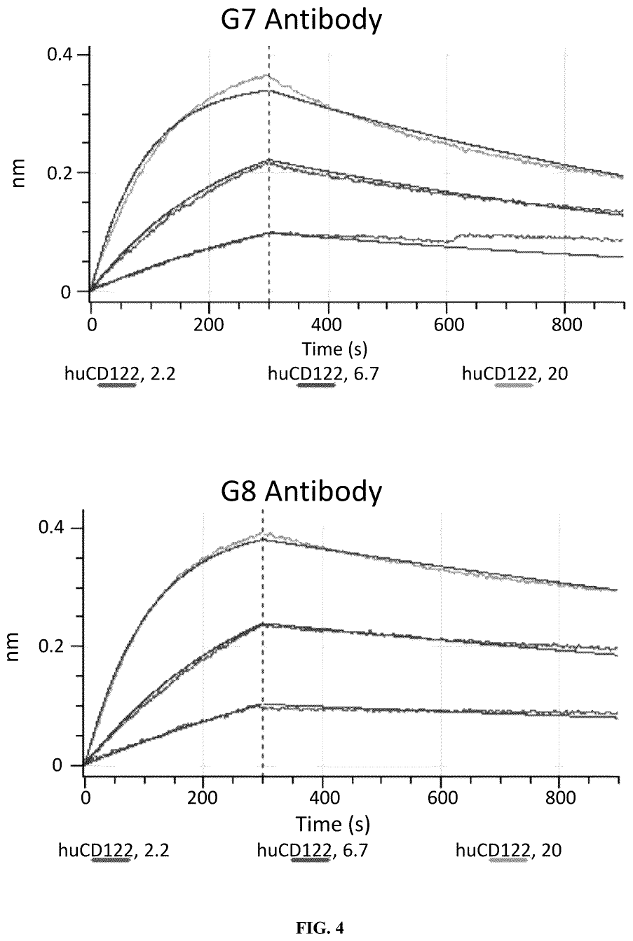

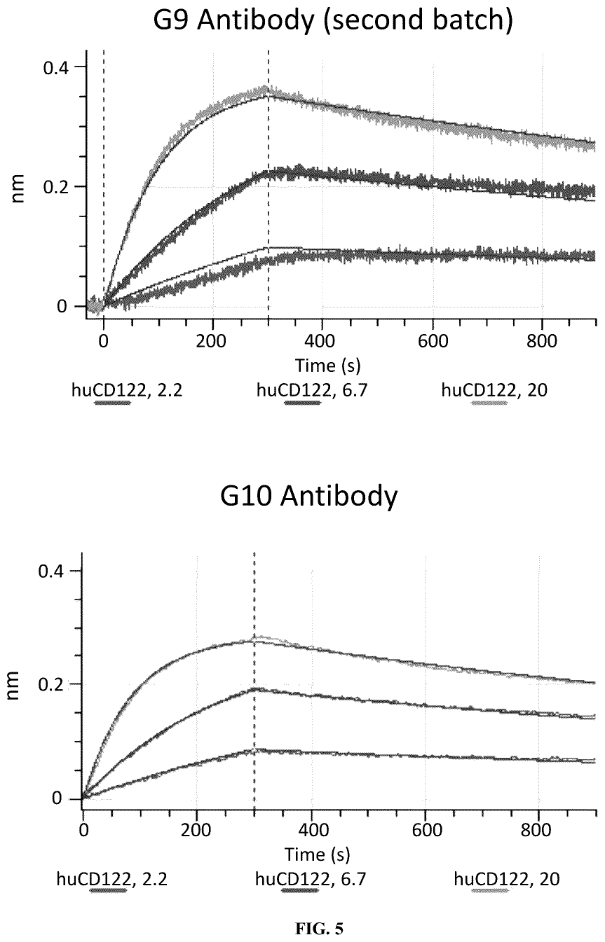

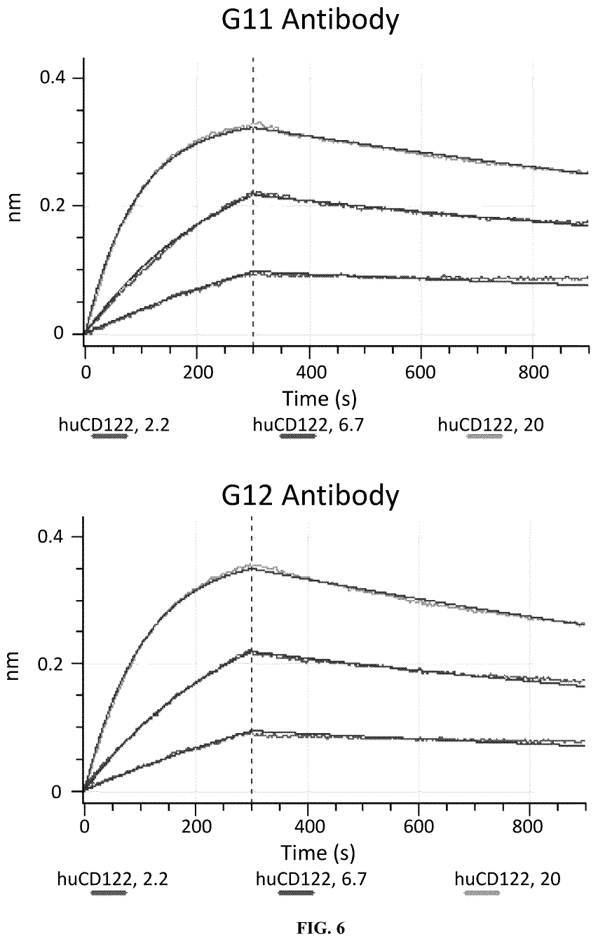

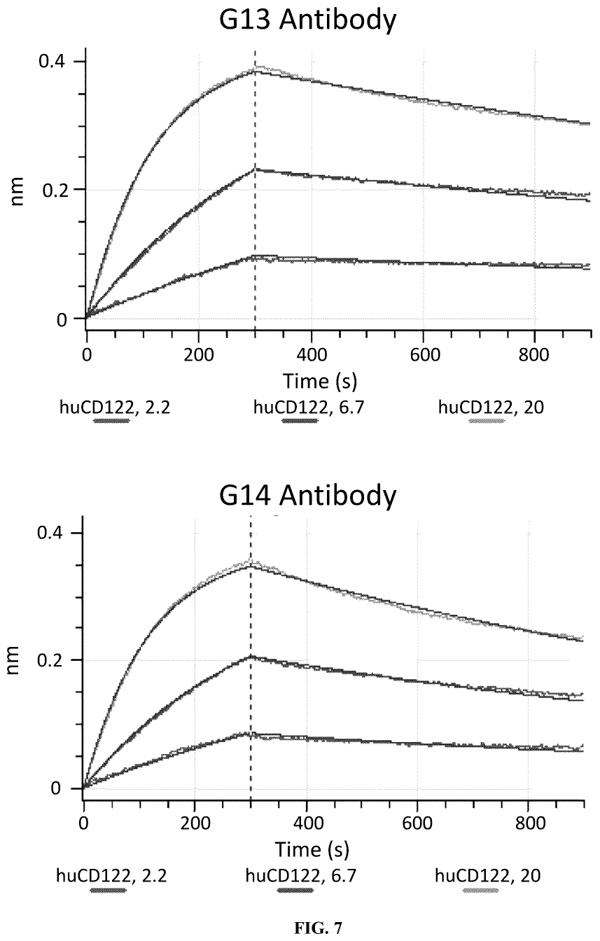

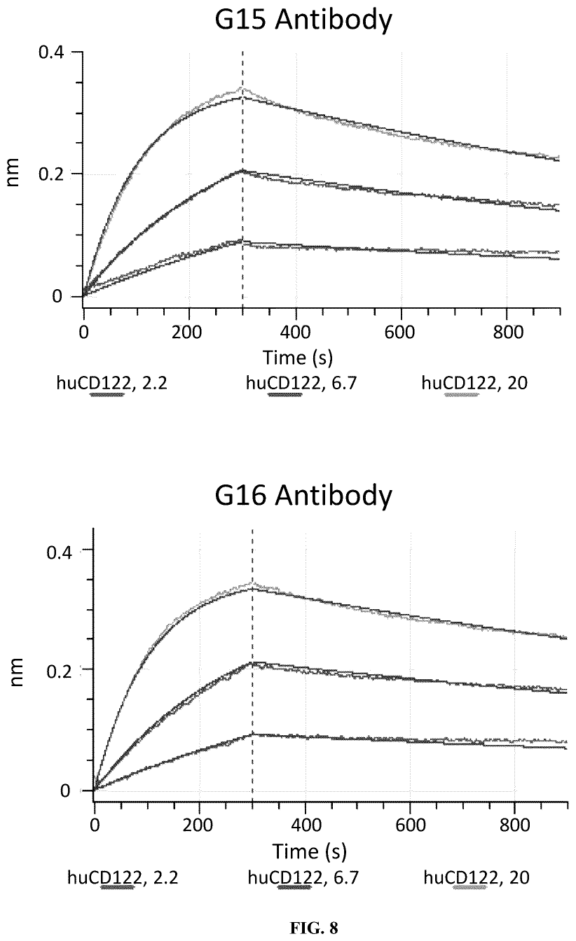

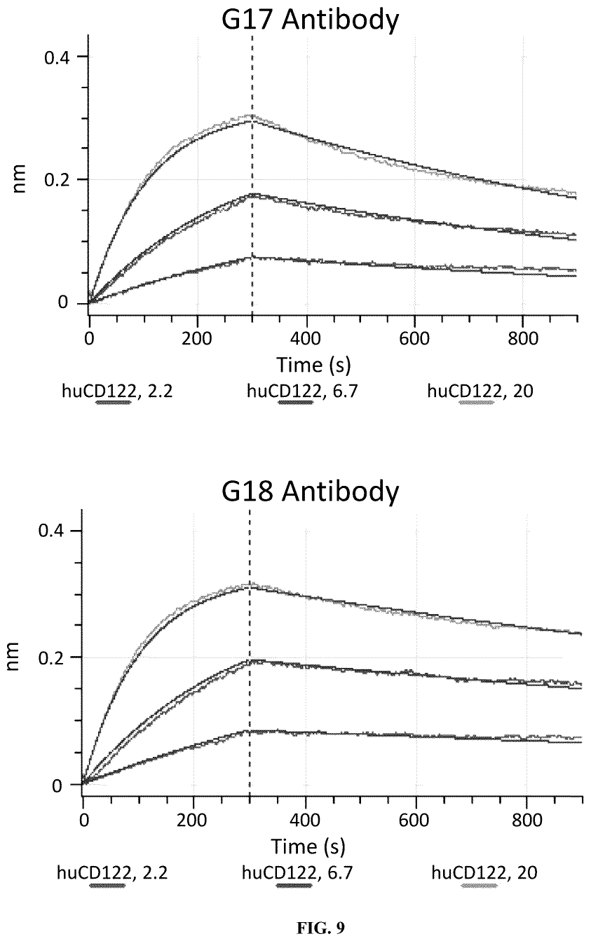

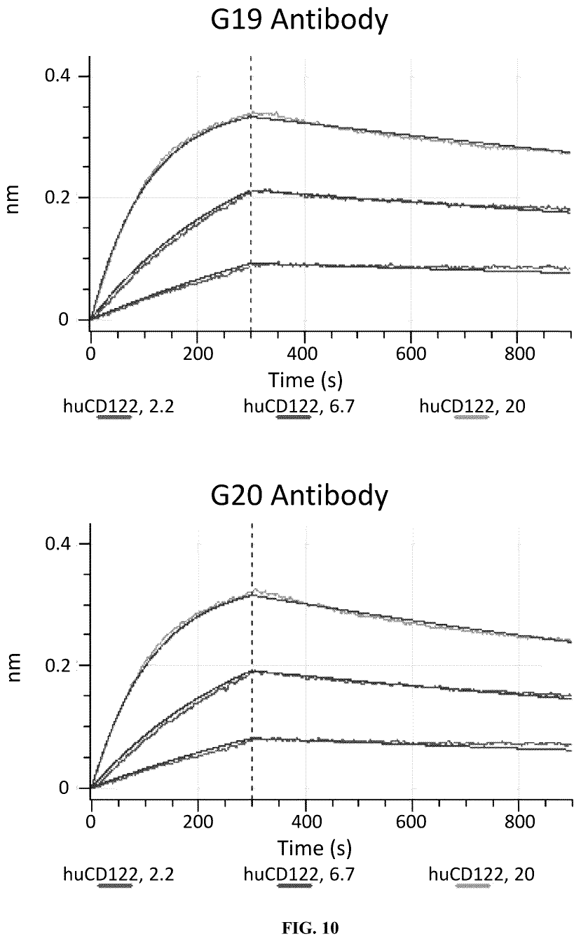

Based on Bio-Layer Interferometry (BLI), the Octet® BLI system is a fluidics-free instrument platform that enables real-time, label-free analysis for the determination of kinetics, affinity and antibody/protein quantitation. Binding experiments were performed on Octet HTX at 25° C. The analyte used was huCD122-HIS (ARCO Bio | Cat No. CD2-H5221) at a molecular weight of 25.4 kDa. The antibodies were loaded onto Anti-human Fc capture (AHC) sensors. The loaded sensors were dipped into serial dilutions of huCD122-His (Starting at 20 nM, 1:3 dilution, 4 points). Reference sample well (buffer) was used for data analysis. Kinetic constants were calculated using a monovalent (1:1) binding model. The assay buffer used was PBS with 0.1% BSA, 5.02% Tween-20, pH 7.2. The regeneration buffer used was 10 mM Glycine buffer (pH 1.7). The four concentrations of huCD122-His used to determine anti-CD122 antibody binding and dissociation properties were 0.0 nM, 2.2 nM, 6.7 nM, and 20 nM. Sensorgram plots to display antibody binding characteristics were generated. The mouse parental chimera configurations in both IgG1 and IgG4 backbones (M5 IgG1 parental and M5 IgG4 parental) were tested as well as each combination of the humanized 3 HC and 3 LC variants in either an IgG1 or an IgG4 backbone. Sensorgram plots for each variant are shown in FIG. 1-FIG. 10.

Binding affinities for antibodies tested are listed in Table 20.

TABLE 20
Binding affinities

| | Loading | | | | | | |
|---|---|---|---|---|---|---|---|
| Antibody | Concentration | | | | | Full | Full |
| Name | (μg/mL) | Response | K_{D} (M) | Kₐ (1/Ms) | K_{dis} (1/s) | X\hat{ }2 | R\hat{ }2 |
| G1 | 5 | 0.3808 | 7.27E⁻¹⁰ | 5.49E⁺⁰⁵ | 4.00E⁻⁰⁴ | 0.4329 | 0.9928 |
| G2 | 5 | 0.3722 | 8.31E⁻¹⁰ | 4.92E⁺⁰⁵ | 4.09E⁻⁰⁴ | 0.382 | 0.9935 |
| G3 | 5 | 0.3939 | 7.54E⁻¹⁰ | 5.24E⁺⁰⁵ | 3.95E⁻⁰⁴ | 0.4995 | 0.9918 |
| G4 | 5 | 0.3635 | 1.67E⁻⁰⁹ | 4.79E⁺⁰⁵ | 8.01E⁻⁰⁴ | 0.3796 | 0.9919 |
| G5 | 5 | 0.3766 | 1.18E⁻⁰⁹ | 4.94E⁺⁰⁵ | 5.81E⁻⁰⁴ | 0.4879 | 0.9908 |
| G6 | 5 | 0.3733 | 8.14E⁻¹⁰ | 5.09E⁺⁰⁵ | 4.15E⁻⁰⁴ | 0.5517 | 0.9901 |
| G7 | 5 | 0.3643 | 1.78E⁻⁰⁹ | 5.21E⁺⁰⁵ | 9.28E⁻⁰⁴ | 0.702 | 0.9834 |
| G8 | 5 | 0.3888 | 9.43E⁻¹⁰ | 4.50E⁺⁰⁵ | 4.25E⁻⁰⁴ | 0.1331 | 0.998 |
| G9 | NA | NA | NA | NA | NA | NA | NA |
| G10 | 5 | 0.2784 | 9.19E⁻¹⁰ | 5.64E⁺⁰⁵ | 5.18E⁻⁰⁴ | 0.0658 | 0.9979 |
| G11 | 5 | 0.3255 | 7.95E⁻¹⁰ | 5.32E⁺⁰⁵ | 4.23E⁻⁰⁴ | 0.0811 | 0.9982 |
| G12 | 5 | 0.3538 | 1.08E⁻⁰⁹ | 4.51E⁺⁰⁵ | 4.88E⁻⁰⁴ | 0.074 | 0.9986 |
| G13 | 5 | 0.3865 | 9.73E⁻¹⁰ | 4.07E⁺⁰⁵ | 3.96E⁻⁰⁴ | 0.0792 | 0.9989 |
| G14 | 5 | 0.3548 | 1.75E⁻⁰⁹ | 3.98E⁺⁰⁵ | 6.97E⁻⁰⁴ | 0.0911 | 0.9982 |
| G15 | 5 | 0.3374 | 1.37E⁻⁰⁹ | 4.66E⁺⁰⁵ | 6.39E⁻⁰⁴ | 0.1642 | 0.9962 |
| G16 | 5 | 0.3417 | 1.03E⁻⁰⁹ | 4.66E⁺⁰⁵ | 4.78E⁻⁰⁴ | 0.1326 | 0.9973 |
| G17 | 5 | 0.3046 | 2.18E⁻⁰⁹ | 4.24E⁺⁰⁵ | 9.22E⁻⁰⁴ | 0.1774 | 0.9948 |
| G18 | 5 | 0.3146 | 9.80E⁻¹⁰ | 4.62E⁺⁰⁵ | 4.53E⁻⁰⁴ | 0.1428 | 0.9967 |
| G19 | 5 | 0.3365 | 6.98E⁻¹⁰ | 4.62E⁺⁰⁵ | 3.22E⁻⁰⁴ | 0.1154 | 0.9978 |
| G20 | 5 | 0.3184 | 1.12E⁻⁰⁹ | 4.15E⁺⁰⁵ | 4.66E⁻⁰⁴ | 0.1298 | 0.9971 |

Second Batch

| | | | | | | | |
|---|---|---|---|---|---|---|---|
| G9 | 5 | 0.3596 | 8.69E⁻¹⁰ | 4.78E⁺⁰⁵ | 4.15E⁻⁰⁴ | 0.6504 | 0.9889 |
| G10 | 5 | 0.3599 | 1.41E⁻⁰⁹ | 4.31E⁺⁰⁵ | 6.07E⁻⁰⁴ | 0.8243 | 0.9845 |
| G11 | 5 | 0.327 | 8.66E⁻¹⁰ | 5.67E⁺⁰⁵ | 4.91E⁻⁰⁴ | 0.4499 | 0.9898 |
| G12 | 5 | 0.3339 | 1.43E⁻⁰⁹ | 4.52E⁺⁰⁵ | 6.46E⁻⁰⁴ | 0.662 | 0.986 |
| G13 | 25 | 0.2534 | 9.95E⁻¹⁰ | 6.48E⁺⁰⁵ | 6.45E⁻⁰⁴ | 0.5525 | 0.9793 |
| G14 | 5 | 0.3008 | 2.57E⁻⁰⁹ | 4.15E⁺⁰⁵ | 1.07E⁻⁰³ | 0.4299 | 0.9875 |
| G15 | 5 | 0.3124 | 1.33E⁻⁰⁹ | 4.85E⁺⁰⁵ | 6.47E⁻⁰⁴ | 0.4598 | 0.9877 |
| G16 | 5 | 0.2511 | 1.18E⁻⁰⁹ | 5.41E⁺⁰⁵ | 6.40E⁻⁰⁴ | 0.4636 | 0.9828 |
| G19 | 5 | 0.3351 | 9.15E⁻¹⁰ | 5.34E⁺⁰⁵ | 4.89E⁻⁰⁴ | 0.3328 | 0.9928 |

### Results:

Comparing the binding affinities for the various HC and LC paired antibodies (G1-G20), many of the humanized versions displayed binding affinities close to that of the parental chimera formats. For instance, the equilibrium dissociation constant (KD) of G1 mouse parental IgG1 chimera measured at 7.27E⁻¹⁰. KD values for the humanized IgG1 variants (G2-G10) ranged from 1.78E⁻⁰⁹ to 7.54E⁻¹⁰. The measured KD values for G11 mouse parental IgG4 chimera were 7.95E⁻¹⁰ and 8.66E⁻¹⁰ (second batch). KD values for the humanized IgG4 variants (G12-G20) ranged from 2.57E⁻⁰⁹ to 6.98E⁻¹⁰. Robust production yields of all antibody variants were also achieved. These features, in addition to the strong T20 humanness score ascribed to each variant VH or VL domain, indicated high-affinity humanized versions of anti-CD122 antibodies with a low likelihood of immunogenic complications when used in a human subject.

### Example 2: Inhibition of IL2-Mediated Proliferation In Vitro by Humanized Anti-CD122 Antibodies

The ability of humanized anti-CD122 antibodies to inhibit proliferation in vitro is tested. A human cell line, TF-1 (ATCC, Manassas, VA), originally established from the bone marrow cells of a subject with erythroleukemia is dependent on the addition of exogenous cytokines such as Erythropoietin (EPO), Granulocyte-macrophage colony-stimulating factor (GM-CSF), or Interleukin 3 (IL3) to culture media for growth. TF-1 expresses the common γ chain IL receptor (CD132), but not the IL2/IL15 receptor 3 chain (CD122). Expression of a CD122-expressing gene in TF-1 allows for the expression of the intermediate and high affinity IL2 and IL15 receptors. TF-1-CD122 cells are generated by transfecting TF-1 with a mammalian expression vector carrying a gene coding for human CD122 and a puromycin resistance gene.

The ability of anti-CD122 monoclonal antibody variants to inhibit cell proliferation mediated by IL2 is examined using TF-CD122 cells. Prior to experiments, TF-CD122 cells are cultured in RPMI 1640 (ThermoFisher Scientific, Cat #11875093) supplemented with 10% heat-inactivated FBS, 50 IU/mL IL2, 2 mM 1-glutamine, 50 U/ml penicillin, and 50 μg/ml streptomycin. Cells are maintained at 37° C. under a humidified 5% C02 atmosphere. In atypical experiment, TF-1-CD122 cells are first deprived of IL2 for 2 days. After that, about 10⁴ cells per well were incubated with serial dilutions of anti-CD122 monoclonal antibody variants (GT-G20) at concentrations between 0.1-10.0 g/mL. Control wells include three control conditions: 1) no antibody plus 50 IU/mL IL2, 2) InVivoMAb human IgG1 isotype control (BioXCell, NH; Catalog #BE0297) at concentrations matching the serial dilutions, or 3) Human IgG4 kappa (S228P) Isotype Control—CrownVivo™ Antibody (MBL International Corp, MA; Catalog #C0045) at concentrations matching the serial dilutions. Treatments with anti-CD122 monoclonal antibody variants GT-G20 (or control conditions) are maintained for 10 minutes at 37° C. and then 50 IU/mL IL2 is added to the culture media. Cells are then cultured for 48 hours. Next, 20 μL per well of alamarBlue™ Cell Viability Reagent (ThermoFisher Scientific; Cat #DAL1025)) is added, and plates are incubated for 6 hours. After the wells are washed, plates are then read using a spectrophotometer microplate reader at 540 and 620 nm. Proliferation curves are obtained following manufacturer's recommendation.

### Example 3: Inhibition of IL15-Mediated Proliferation In Vitro by Humanized Anti-CD122 Antibodies

The ability of humanized anti-CD122 antibodies to inhibit proliferation in vitro is tested. TF-CD122 cells are cultured in RPMI 1640 (ThermoFisher Scientific, Cat #11875093) supplemented with 10% heat-inactivated FBS, 2 mM 1-glutamine, 50 U/ml penicillin, and 50 μg/ml streptomycin. To simulate presentation of IL15 in-trans, a soluble complex of human IL15 bound to a portion of the extracellular region of the human IL15Ra (scIL15/IL15Rα) is constructed following (Mortier et al., 2006; J. Biol. Chem., 281:1612-1619) and added to cell culture medium at a concentration of 10 nM. Cellular proliferation is assayed to determine if scIL15/IL15Ra at 10 nM can support growth of TF-CD122 cells. Cells are maintained at 37° C. under a humidified 5% C02 atmosphere. In a typical experiment, TF-1-CD122 cells are first deprived of scIL15/IL15Ra for 2 days. After that, about 10⁴ cells per well were incubated with serial dilutions of anti-CD122 monoclonal antibody variants (GT-G20) at concentrations between 0.1-10.0 g/mL. Control wells include three control conditions: 1) no antibody plus 10 nM scIL15/IL15Rα, 2) InVivoMAb human IgG1 isotype control (BioXCell, NH; Catalog #BE0297) at concentrations matching the serial dilutions, or 3) Human IgG4 kappa (S228P) Isotype Control—CrownVivo™ Antibody (MBL International Corp, MA; Catalog #C0045) at concentrations matching the serial dilutions. Treatments with anti-CD122 monoclonal antibody variants GT-G20 (or control conditions) are maintained for 10 minutes at 37° C. and then 10 nM scIL15/IL15Ra is added to the culture media. Cells are then cultured for 48 hours. Next, 20 μL per well of alamarBlue™ Cell Viability Reagent (ThermoFisher Scientific; Cat #DAL1025)) is added, and plates are incubated for 6 hours. After the wells are washed, plates are then read using a spectrophotometer microplate reader at 540 and 620 nm. Proliferation curves are obtained following manufacturer's recommendation.

### Example 4: Inhibition of IL2 and IL15 Signaling by Anti-CD122 Antibodies

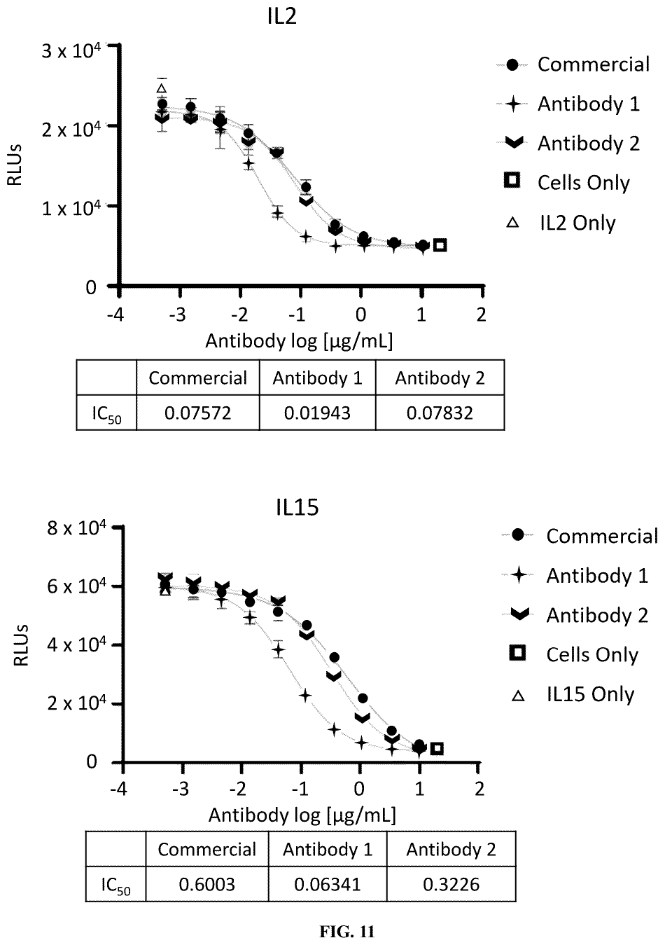

Several anti-CD122 antibodies described herein were tested using in vitro assays for their ability to inhibit IL2 and/or IL15 signaling. A reporter cell line expressing the beta gamma receptor (CD122/CD132) for IL2 and IL15, which has intermediate affinity for each IL2 and IL15, and luciferase under the regulation of IL2 and IL15 binding to its receptor was used to test the ability of anti-CD122 antibodies to inhibit IL2 and/or IL15 signaling. In FIG. 11, three anti-CD122 antibodies were assayed for their ability to inhibit IL2 and/or IL15 signaling. The reporter cells were treated with either IL2 (12.5 ng/mL) or IL15 (6 ng/mL) and also treated with an anti-CD122 antibody at ten different antibody concentrations ranging from 10 μg/mL to 0.5 ng/mL. The anti-CD122 antibody designated as Commercial, which is used as a positive control of the experiment, is the anti-human CD122 antibody Clone TU27, obtained from BioLegend®, Catalog #339015. Antibody 1 and Antibody 2 are humanized anti-human CD122 antibodies. Antibody 1 is a humanized monoclonal antibody described herein comprising CDR sequences of CDR series E7 listed in Table 3. Antibody 2 is a known anti-CD122 humanized monoclonal antibody comprising a VH domain and a VL domain, wherein the VH domain comprises an HCDR1 comprising the sequence of SEQ ID NO: 189, an HCDR2 comprising the sequence of SEQ ID NO: 190, and an HCDR3 comprising the sequence of SEQ ID NO: 191; and wherein the VL domain comprises an LCDR1 comprising the sequence of SEQ ID NO: 192, an LCDR2 comprising the sequence of SEQ ID NO: 193, and an LCDR3 comprising the sequence of SEQ ID NO: 194.

Results from a negative control for these experiments are shown in the graphs in FIG. 11, wherein the cells were treated with either IL2 only (top graph) or IL15 only (bottom graph) without any anti-CD122 antibody co-treatment and the luminescence signals of the cell-based reporter assays were plotted in relative luminescence unit (RLU) values. Results from another negative control for these experiments are shown in the graphs in FIG. 11, wherein the cells were not treated with IL2 or IL15, nor were they treated with anti-CD122 antibody. Measurements of IL2/IL15 signaling after the co-treatment of IL2 or IL15 (12.5 and 6 ng/mL, respectively) with anti-CD122 antibody (in a dilution series calculated using a dose-response curve, with the tested dose ranging from 10 μg/mL to 1.5 ng/mL) were plotted in RLUs as a function of anti-CD122 antibody concentration (μg/mL in a log scale). The median effective doses (EC₅₀) of IL2 and/or IL15 antagonist (anti-CD122 antibody) for reducing IL2 and/or IL15 signaling in response to IL2 or IL15 treatment were calculated to determine the effects of tested anti-CD122 antibodies. The results in FIG. 11 demonstrate that the Antibody 1 antibody is a more potent inhibitor of IL2 signaling and IL15 signaling than the Commercial antibody or Antibody 2. Antibody 1 was able in this assay to inhibit both IL2 signaling and IL15 signaling 3-5 fold more efficiently than Antibody 2. These results also indicate inhibition of the beta gamma receptor (CD122/CD132) using the tested anti-CD122 antibodies, with Antibody 1 being the most efficient inhibitor.

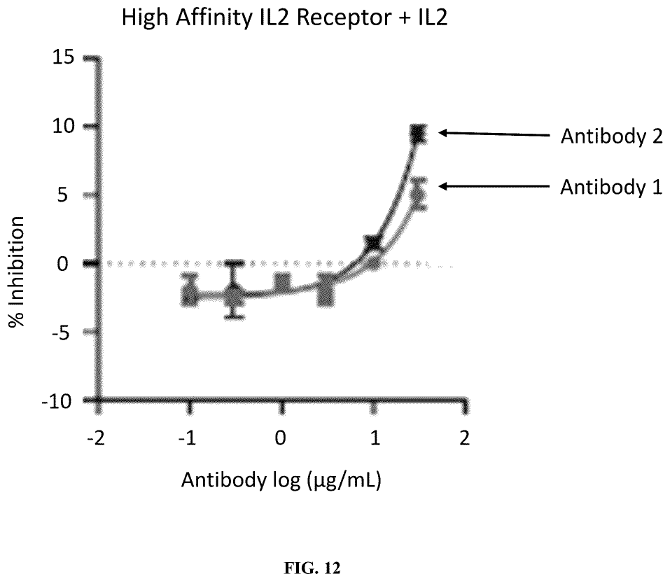

In another in vitro assay, a TF-1αβ cell line expressing the alpha beta gamma receptor (IL2Rα/IL2Rβ/IL2Rγ complex) for IL2, which has high affinity for IL2, was used to test the ability anti-CD122 antibodies to inhibit IL2-mediated cell proliferation. In FIG. 12, cells were treated with IL2 at a dosage of 10 ng/mL and also were treated with an anti-CD122 antibody at six different antibody concentrations ranging from 30 g/mL to 30 ng/mL. The results were plotted as a percentage of inhibition in cell proliferation compared to no antibody treatment over concentration of anti-CD122 antibody. The plotted data indicates that neither Antibody 2 nor Antibody 1 are efficient inhibitors of the alpha beta gamma receptor.

### Example 5: IL2 Interleukin Receptor Beta Epitope Mapping Using Humanized Anti-CD122 Antibody

In this example, the objective was to map the epitope interactions of a humanized monoclonal antibody (Mab) described herein and the human IL2 Interleukin Receptor Beta (IL2RB) using Plasma Induced Modification of Biomolecules (PLIMB) technology.

### Experimental Design and Approach:

PLIMB and MS Optimization: Prior to epitope mapping, the level of antigen modification in response to several PLIMB-induced hydroxyl radical exposure doses was monitored. Samples were prepared, and exposed to PLIMB for 20 and 40 seconds. Samples were prepared as described below and LC-MS/MS was utilized to optimize labeling and digestion coverage for the subsequent quantitative analysis.

IL2RB Epitope Mapping: Differences in solvent accessibility were measured for the antigen (IL2RB) using the trifluoromethyl radical (CF3) produced via PLIMB exposure. First, two solutions were prepared to map the IL2RB epitope. The first contained IL2RB and a non-binding, standard IgG control antibody from NIST. The second contained the humanized anti-CD122 Mab added at a 1:1 antibody/antigen molar ratio. Samples of IL2RB with NIST antibody are referred to as “unbound,” and samples with humanized anti-CD122 Mab added are referred to as “bound” or “complex”. A residue-level analysis was utilized for peptides in the candidate epitope regions to determine which of the individually labeled amino acids show the greatest changes in solvent accessibility due to binding. Individual residues in the epitope regions that show changes in solvent accessibility upon binding represent “hotspots”, which can be used to further pinpoint regions involved in antigen/antibody epitope interactions.

### Results:

### IL2RB Labeling and Coverage

The work described in this example focuses on the R subunit (IL2RB) of the Interleukin-2 receptor (IL2R). IL2RB in this example contains the extracellular domain (A26-D239). After analyzing PLIMB-exposed samples, all regions of IL2RB showed sufficient labeling across the protein sequence at both 20 and 40 s.

### IL2RB Epitope Determination

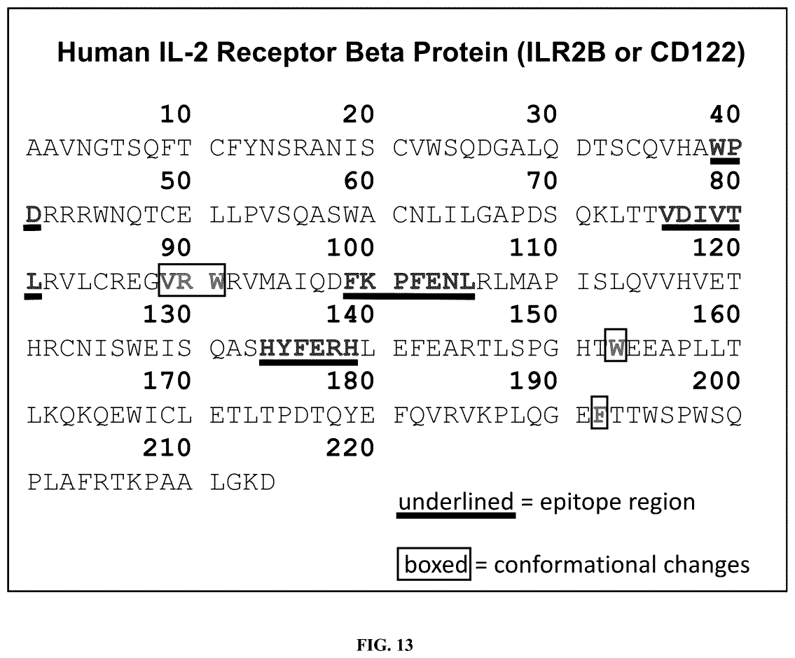

A residue-level analysis was performed across the entire length of the IL2RB primary sequence for PLIMB treated samples prepared in the unbound state with control antibody (NIST) and in the bound state with the addition of the humanized anti-CD122 Mab to detect differences in solvent accessibility (modification level) upon complexation (FIG. 13). FIG. 13 shows annotation/numbering based on the extracellular IL2RB form of the protein used in this example corresponding to [A26-D239] of the canonical sequence (UniProt ID: P14784). Labeling of amino acid residues for epitope mapping in this Example is according to amino acid position within the human CD122 polypeptide sequence of SEQ ID NO: 187 (e.g., W39 corresponds to position 39 in SEQ ID NO: 187 and also to position 64 in SEQ ID NO: 186). Residues in bold exhibited significant changes in solvent accessibility (unlabeled residues between significantly changing residues were included to indicate epitope regions). Underlined residues indicate epitope regions. Boxed residues (contained within a box) indicate conformational changes.

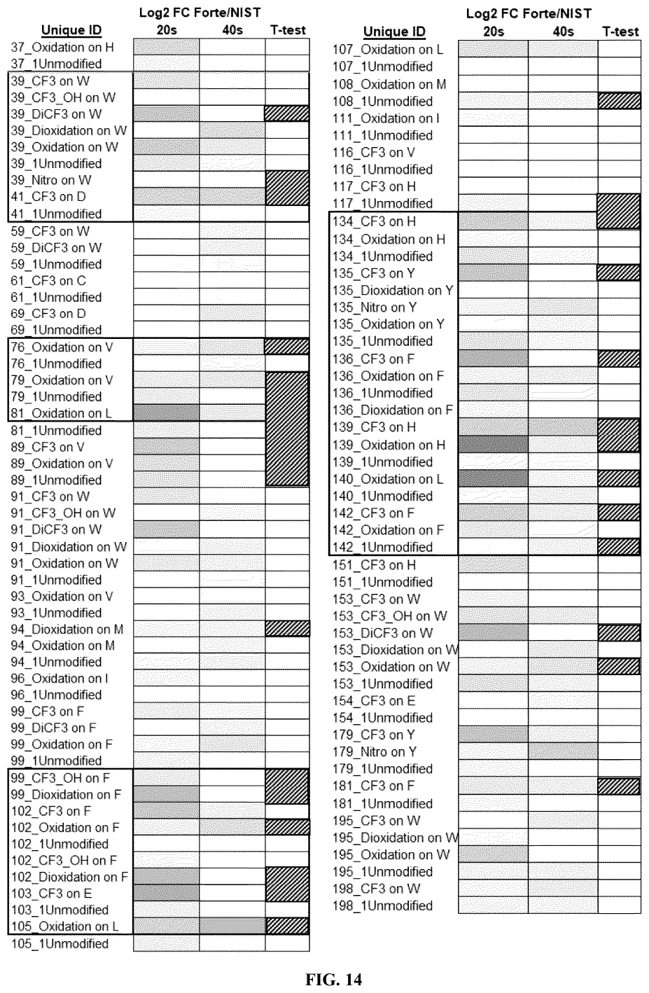

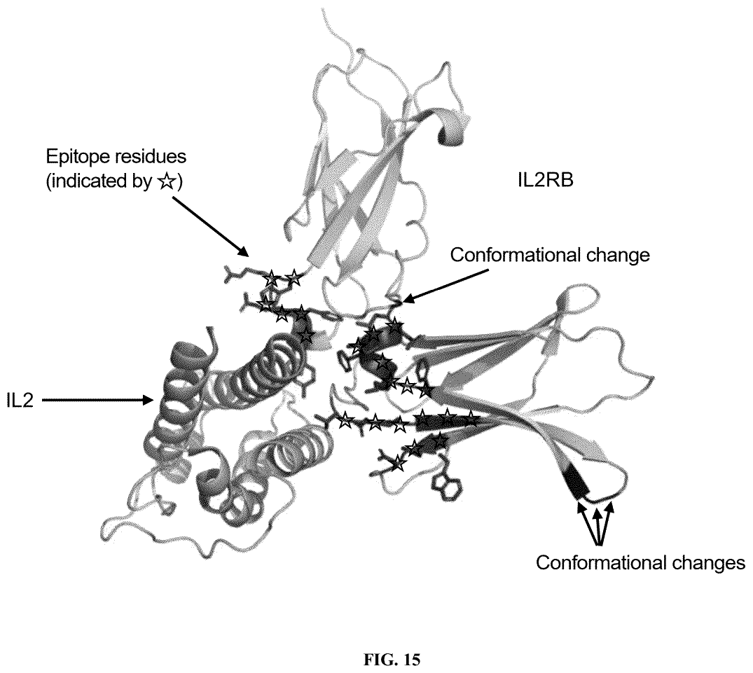

Changes in solvent accessibility were mapped onto the IL2RB crystal structure and displayed as fold changes with statistical significance (FIG. 15 and FIG. 14). Protection, or decreases in labeling in the experimental antibody compared to control, are indications of epitope interactions. FIG. 14 shows a heatmap of modification changes in humanized anti-CD122 Mab bound versus unbound IL2RB. The log 2 fold change was of the humanized anti-CD122 Mab (bound) over the NIST control antibody (unbound) incubated with IL2RB at 20 and 40 second PLIMB time points. Green indicates a decrease and red indicates an increase. Significant changes (p<0.05) are indicated by hatched boxes in the t-test column.

High resolution footprinting analysis via PLIMB-induced CF3 radical labeling revealed multiple residue “hotspots” on IL2RB in the antibody “bound” condition. These “hotspots” were mapped onto the 3D crystal structure of IL2RB (FIG. 15). Conformational epitope regions are expected to cluster together in a specific region of the protein. From the 3D crystal structure mapping, four specific regions were identified as epitope sites: 1) residues 39-41 (WPD), 2) residues 76-81 (VDIVTL (SEQ ID NO: 195)), 3) residues 99-105 (FKPFENL (SEQ ID NO: 196)), and 4) residues 134-139 (HYFERH (SEQ ID NO: 197)). These regions all map to the IL2 binding interface in the 3D crystal structure (FIG. 15). Additionally, these regions show relatively consistent changes at both the 20 and 40s PLIMB exposure times, which indicates more stable interactions that are indicative of epitope/antibody binding regions.

Additional “hotspots” were mapped to areas outside of the IL2 binding interface. These “hotspots” are consistent with distal conformational changes and not epitope regions. This determination was made based on their relative degree of change, which is generally smaller than sites involved in direct antibody binding, and their relative isolation from other “hotspot” clusters. Positions mapped as sites of conformational changes include: 1) residues 89-91 (VRW), 2) residue 153 (W), and 3) residue 192 (F).

In conclusion, distinct residues have been identified involved in the binding of the humanized anti-CD122 antibody to IL2RB which are primarily located on the IL2 binding face of IL2RB.

### Method Overview:

Sample preparation, PLIMB treatment and LC-MS/MS Analysis: The humanized anti-CD122 Mab and NIST antibody were incubated with IL2RB at room temperature for one hour to promote binding. For trifluoromethylation labeling (CF3), samples were exposed to PLIMB treatment for 20 and 40 seconds in the presence of 50 mM sodium triflinate and 10 mM hydrogen peroxide (3-5 replicates per condition). Following labeling, samples were quenched with a 5 μL solution of 250 mM methionine in PBS (pH 7.4).

Following PLIMB exposure, samples were proteolytically digested into peptides with a sequential digest of trypsin and elastase. Samples were subjected to solid phase extraction using C18 StageTips, labeled with 6-plex tandem mass tags (Thermo Scientific TMT 6-plex), and then analyzed using data-dependent acquisition with an Orbitrap Exploris 240 mass spectrometer.

### Data Analysis:

The ‘.raw’ data files were searched against the IL2RB sequence using the database search engine MetaMorpheus. A list of standard expected modifications and expected PLIMB modifications was utilized in the database search. Peptides were identified using MS and MS/MS spectra, setting a 1% false discovery rate (FDR) cutoff. Changes in solvent accessibility for both the trypsin and trypsin-elastase digested samples were determined via comparison of the sum normalized TMT channel intensities for the control antibody versus the humanized anti-CD122 target Mab. Reported solvent accessibility changes were derived from the trypsin-elastase digested samples. These changes were verified via examination of the trypsin digested samples.

### Statistical Analysis:

The fold-change was calculated for IL2RB peptides in the humanized anti-CD122 Mab bound and unbound states and a student's t-test was performed for each peptide. p<0.05 was considered a significant change.

### Summary of Experimental Conditions:

Antigen Sample: Human IL2RB(A26-D239), His Tag (Acro Biosystems)
Antigen concentration: 0.1 mg/mL (2.7 μM)
Control Antibody (NIST) concentration: 0.42 mg/mL (2.7 μM)
Experimental Antibody concentration: 0.42 mg/mL (2.7 μM)
Antibody/antigen ratio: 1:1
Experimental sample volume: 50 μL
Sample buffer: 100 mM PBS, pH 7.4
Protease(s) used: Trypsin Platinum (Promega) and Elastase (Promega)
Instrument: Thermo Orbitrap Exploris 240
Data processing software: MetaMorpheus

### Detailed Methods:

### Sample Preparation and Digestion:

Following PLIMB treatment, samples were denatured and reduced with 8 M guanidine HCl (GnHCl) containing 5 mM TCEP. The samples were heated to 90° C. for 15 minutes and then cooled. Samples were alkylated with 15 mM IAA for 30 minutes at room temperature in the dark. The samples were diluted to 1.5 M GnHCl and subjected to overnight trypsin (Promega) digestion at a total protease/protein mass ratio of 1:10 at 37° C. Reactions were quenched by addition of 2% trifluroacetic acid prior to desalting and clean-up using Empore C18 StageTips (CDS Analytical) using the standard protocol. Half of the eluted peptides were dried with a vacuum concentrator and resuspended in 0.1% formic acid for LC-MS/MS analysis. Remaining samples were resuspended in 100 mM Tris-HCl for secondary digestion with elastase (Promega) at a ratio of 1:20 for 4 hours at 37° C. After quenching, peptides were desalted, dried down, and resuspended in 50 mM HEPES buffer at pH 8.5. Samples were labeled with tandem mass tags (6-plex TMT, ThermoFisher) according to the manufacturer's protocol.

### LC-MS/MS Acquisition

A 60-minute chromatographic gradient from 2 to 40% acetonitrile with 0.1% formic acid was used for separation over a 2 μM, 15 cm Easy-Spray PepMap C18 column from ThermoFisher Scientific. A top-10 data-dependent acquisition was performed MS1 parameters of 60K resolving power in the Orbitrap, a scan range of 350-1200 m/z, a normalized AGC target of 300%, and MS² parameters of charge state 1-6 selection, a quadrupole isolation window of 2 Da, HCD stepped collision energy of 28, 36, 42%, a normalized AGC value of 50%, and an automatic scan range starting at 110 m/z. Dynamic exclusion of 6 seconds was used after seeing an ion once.

### Data Analysis:

The following modifications were used in our MetaMorpheus search:

Standard modifications:
Carbamidomethyl/+57.021464@C (fixed)

### PLIMB Modifications:

Oxidation/+15.994915@C, F, H, I, L, M, W, Y
Dioxidation/+31.989829@C, F, M, W, Y
Cys-Oxidation/+15.994915-57.021464@C
Cys-Dioxidation/+31.989829-57.021464@C
Cys-Trioxidation/+47.984745-57.021464@C
Nitro/+44.985078@W, Y
Trifluoromethylation/+67.9874@A, C, D, E, F, G, H, I, K, L, M, N, P, Q, R, S, T, V, W, Y
Di-Trifluoromethylation/+135.9748@A, C, D, E, F, G, H, I, K, L, M, N, P, Q, R, S, T, V, W, Y
Cys-Trifluoromethylation/+67.9874-57.021464@C

### Summary of Results:

Epitope of Human Interleukin 2 Receptor Beta (IL2RB) with humanized anti-CD122 Mab was mapped using PLIMB-generated hydroxyl (OH) and trifluoromethyl (CF3) labelling. The resulting epitope is conformational rather than linear. The mapped epitope is contained within the following regions: W39-D41, V76-L81, F99-L105, and H134-H139. These cluster over a distinct interface of the IL2RB protein. The mapped conformational epitope covers a region that overlaps significantly with the IL2/IL2RB binding interface, suggesting the antibody would be an effective IL2RB antagonist.

While preferred aspects of the present invention have been shown and described herein, it will be obvious to those skilled in the art that such aspects are provided by way of example only. Numerous variations, changes, and substitutions will now occur to those skilled in the art without departing from the invention. It should be understood that various alternatives to the aspects of the invention described herein may be employed in practicing the invention. It is intended that the following claims define the scope of the invention and that methods and structures within the scope of these claims and their equivalents be covered thereby.

### Aspects

Aspect 1: An antibody that binds to CD122 comprising:
an HCDR1 comprising the amino acid sequence of SEQ ID NO: 7; an HCDR2 comprising the amino acid sequence of SEQ ID NO: 18; an HCDR3 comprising the amino acid sequence of SEQ ID NO: 30; an LCDR1 comprising the amino acid sequence of SEQ ID NO: 43; an LCDR2 comprising the amino acid sequence of YTS; and an LCDR3 comprising the amino acid sequence of SEQ ID NO: 62.

Aspect 2: An antibody or antigen-binding fragment thereof that binds to CD122 comprising:
an HCDR1 having the sequence of SEQ ID NO: 7, an HCDR2 having the sequence of SEQ ID NO: 18, an HCDR3 having the sequence of SEQ ID NO: 30, an LCDR1 having the sequence of SEQ ID NO: 43, an LCDR2 having the sequence of YTS, and an LCDR3 having the sequence of SEQ ID NO: 62, according to the IMGT antibody numbering scheme.

Aspect 3: An antibody or antigen-binding fragment thereof that binds to CD122 comprising:
an HCDR1 having the sequence of SEQ ID NO: 162, an HCDR2 having the sequence of SEQ ID NO: 169, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the Kabat antibody numbering scheme.

Aspect 4: An antibody or antigen-binding fragment thereof that binds to CD122 comprising:
an HCDR1 having the sequence of SEQ ID NO: 163, an HCDR2 having the sequence of SEQ ID NO: 170, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the AbM antibody numbering scheme.

Aspect 5: An antibody or antigen-binding fragment thereof that binds to CD122 comprising:
an HCDR1 having the sequence of SEQ ID NO: 164, an HCDR2 having the sequence of SEQ ID NO: 171, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the Chothia antibody numbering scheme.

Aspect 6: An antibody or antigen-binding fragment thereof that binds to CD122 comprising:
an HCDR1 having the sequence of SEQ ID NO: 165, an HCDR2 having the sequence of SEQ ID NO: 172, an HCDR3 having the sequence of SEQ ID NO: 177, an LCDR1 having the sequence of SEQ ID NO: 179, an LCDR2 having the sequence of SEQ ID NO: 182, and an LCDR3 having the sequence of SEQ ID NO: 184, according to the Contact antibody numbering scheme.

Aspect 7: An antibody or antigen-binding fragment thereof that binds to CD122 comprising:
an HCDR1 having the sequence of SEQ ID NO: 166, an HCDR2 having the sequence of SEQ ID NO: 173, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the Kabat antibody numbering scheme.

Aspect 8: An antibody or antigen-binding fragment thereof that binds to CD122 comprising:
an HCDR1 having the sequence of SEQ ID NO: 167, an HCDR2 having the sequence of SEQ ID NO: 170, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the AbM antibody numbering scheme.

Aspect 9: An antibody or antigen-binding fragment thereof that binds to CD122 comprising:
an HCDR1 having the sequence of SEQ ID NO: 164, an HCDR2 having the sequence of SEQ ID NO: 171, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the Chothia antibody numbering scheme.

Aspect 10: An antibody or antigen-binding fragment thereof that binds to CD122 comprising:
an HCDR1 having the sequence of SEQ ID NO: 168, an HCDR2 having the sequence of SEQ ID NO: 174, an HCDR3 having the sequence of SEQ ID NO: 177, an LCDR1 having the sequence of SEQ ID NO: 179, an LCDR2 having the sequence of SEQ ID NO: 182, and an LCDR3 having the sequence of SEQ ID NO: 184, according to the Contact antibody numbering scheme.

Aspect 11: An antibody or antigen-binding fragment thereof that binds to CD122 comprising:
an HCDR1 having the sequence of SEQ ID NO: 166, an HCDR2 having the sequence of SEQ ID NO: 173, an HCDR3 having the sequence of SEQ ID NO: 176, an LCDR1 having the sequence of SEQ ID NO: 180, an LCDR2 having the sequence of SEQ ID NO: 183, and an LCDR3 having the sequence of SEQ ID NO: 62, according to the Kabat antibody numbering scheme.

Aspect 12: An antibody or antigen-binding fragment thereof that binds to CD122 comprising:
an HCDR1 having the sequence of SEQ ID NO: 167, an HCDR2 having the sequence of SEQ ID NO: 170, an HCDR3 having the sequence of SEQ ID NO: 176, an LCDR1 having the sequence of SEQ ID NO: 180, an LCDR2 having the sequence of SEQ ID NO: 183, and an LCDR3 having the sequence of SEQ ID NO: 62, according to the AbM antibody numbering scheme.

Aspect 13: An antibody or antigen-binding fragment thereof that binds to CD122 comprising:
an HCDR1 having the sequence of SEQ ID NO: 164, an HCDR2 having the sequence of SEQ ID NO: 171, an HCDR3 having the sequence of SEQ ID NO: 176, an LCDR1 having the sequence of SEQ ID NO: 180, an LCDR2 having the sequence of SEQ ID NO: 183, and an LCDR3 having the sequence of SEQ ID NO: 62, according to the Chothia antibody numbering scheme.

Aspect 14: An antibody or antigen-binding fragment thereof that binds to CD122 comprising:
an HCDR1 having the sequence of SEQ ID NO: 168, an HCDR2 having the sequence of SEQ ID NO: 174, an HCDR3 having the sequence of SEQ ID NO: 177, an LCDR1 having the sequence of SEQ ID NO: 179, an LCDR2 having the sequence of SEQ ID NO: 182, and an LCDR3 having the sequence of SEQ ID NO: 184, according to the Contact antibody numbering scheme.

Aspect 15: An antibody or antigen-binding fragment thereof that binds to CD122 comprising:
an HCDR1 having the sequence of SEQ ID NO: 162, an HCDR2 having the sequence of SEQ ID NO: 175, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the Kabat antibody numbering scheme.

Aspect 16: An antibody or antigen-binding fragment thereof that binds to CD122 comprising:
an HCDR1 having the sequence of SEQ ID NO: 163, an HCDR2 having the sequence of SEQ ID NO: 170, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the AbM antibody numbering scheme.

Aspect 17: An antibody or antigen-binding fragment thereof that binds to CD122 comprising:
an HCDR1 having the sequence of SEQ ID NO: 164, an HCDR2 having the sequence of SEQ ID NO: 171, an HCDR3 having the sequence of SEQ ID NO: 176, an LCDR1 having the sequence of SEQ ID NO: 178, an LCDR2 having the sequence of SEQ ID NO: 181, and an LCDR3 having the sequence of SEQ ID NO: 62, according to the Chothia antibody numbering scheme.

Aspect 18: An antibody or antigen-binding fragment thereof that binds to CD122 comprising:
an HCDR1 having the sequence of SEQ ID NO: 165, an HCDR2 having the sequence of SEQ ID NO: 174, an HCDR3 having the sequence of SEQ ID NO: 177, an LCDR1 having the sequence of SEQ ID NO: 179, an LCDR2 having the sequence of SEQ ID NO: 182, and an LCDR3 having the sequence of SEQ ID NO: 184, according to the Contact antibody numbering scheme.

Aspect 19: An antibody or antigen-binding fragment thereof that binds to CD122 comprising:
an HCDR1 having the sequence of SEQ ID NO: 162, an HCDR2 having the sequence of SEQ ID NO: 175, an HCDR3 having the sequence of SEQ ID NO: 176, an LCDR1 having the sequence of SEQ ID NO: 180, an LCDR2 having the sequence of SEQ ID NO: 183, and an LCDR3 having the sequence of SEQ ID NO: 62, according to the Kabat antibody numbering scheme.

Aspect 20: An antibody or antigen-binding fragment thereof that binds to CD122 comprising:
an HCDR1 having the sequence of SEQ ID NO: 163, an HCDR2 having the sequence of SEQ ID NO: 170, an HCDR3 having the sequence of SEQ ID NO: 176, an LCDR1 having the sequence of SEQ ID NO: 180, an LCDR2 having the sequence of SEQ ID NO: 183, and an LCDR3 having the sequence of SEQ ID NO: 62, according to the AbM antibody numbering scheme.

Aspect 21: An antibody or antigen-binding fragment thereof that binds to CD122 comprising:
an HCDR1 having the sequence of SEQ ID NO: 164, an HCDR2 having the sequence of SEQ ID NO: 171, an HCDR3 having the sequence of SEQ ID NO: 176, an LCDR1 having the sequence of SEQ ID NO: 180, an LCDR2 having the sequence of SEQ ID NO: 183, and an LCDR3 having the sequence of SEQ ID NO: 62, according to the Chothia antibody numbering scheme.

Aspect 22: An antibody or antigen-binding fragment thereof that binds to CD122 comprising:
an HCDR1 having the sequence of SEQ ID NO: 165, an HCDR2 having the sequence of SEQ ID NO: 174, an HCDR3 having the sequence of SEQ ID NO: 177, an LCDR1 having the sequence of SEQ ID NO: 179, an LCDR2 having the sequence of SEQ ID NO: 182, and an LCDR3 having the sequence of SEQ ID NO: 184, according to the Contact antibody numbering scheme.

Aspect 23: The antibody of any one of aspects 1-22, wherein the antibody is a monoclonal antibody.

Aspect 24: The antibody of any one of aspects 1-23, wherein the antibody is a humanized monoclonal antibody.

Aspect 25: The antibody of any one of aspects 1-23, wherein the antibody is a human antibody.

Aspect 26: The antibody or antigen binding fragment thereof of any one of aspects 1-25, wherein the CD122 is a mammalian CD122.

Aspect 27: The antibody or antigen binding fragment thereof of any one of aspects 1-26, wherein the CD122 is mouse CD122.

Aspect 28: The antibody or antigen binding fragment thereof of any one of aspects 1-26, wherein the CD122 is human CD122.

Aspect 29: The antibody of any one of aspects 1-28, wherein the antibody binds to human CD122 protein with a binding affinity in the nanomolar range (K_{D} value of 10⁻⁷ to 10⁻⁹ M).

Aspect 30: The antibody of any one of aspects 1-28, wherein the antibody binds to human CD122 protein with a binding affinity in the low nanomolar range (K_{D} value of 10⁻⁹ M).

Aspect 31: The antibody of any one of aspects 1-28, wherein the antibody binds to human CD122 protein with a binding affinity in the picomolar range (K_{D} value of 10⁻¹⁰ to 10⁻¹² M).

Aspect 32: The antibody of any one of aspects 1-28, wherein the antibody binds to human CD122 protein with a binding affinity in the high picomolar range (K_{D} value of 10⁻¹⁰ M).

Aspect 33: The antibody of any one of aspects 1-28, wherein the antibody binds to human CD122 protein with a binding affinity falling within the low nanomolar range to the high picomolar range (K_{D} value of 10⁻⁹ to 10⁻¹⁰ M).

Aspect 34: The antibody of any one of aspects 1-28, wherein the antibody binds to human CD122 protein with a binding affinity as measured by K_{D} of less than about 3E⁻⁰⁹ M.

Aspect 35: The antibody of any one of aspects 1-28, wherein the antibody binds to human CD122 protein with a binding affinity as measured by K_{D} of less than about 2E⁻⁰⁹ M.

Aspect 36: The antibody of any one of aspects 1-28, wherein the antibody binds to human CD122 protein with a binding affinity as measured by K_{D} of less than about 1E⁻⁰⁹ M.

Aspect 37: The antibody of any one of aspects 1-28, wherein the antibody binds to human CD122 protein with a binding affinity as measured by K_{D} of less than about 9E⁻¹⁰ M.

Aspect 38: The antibody of any one of aspects 1-28, wherein the antibody binds to human CD122 protein with a binding affinity as measured by K_{D} of less than about 8E⁻¹⁰ M.

Aspect 39: The antibody of any one of aspects 1-28, wherein the antibody binds to human CD122 protein with a binding affinity as measured by K_{D} of between about 5E⁻⁰⁹ to 5E⁻¹⁰ M.

Aspect 40: The antibody of any one of aspects 1-28, wherein the antibody binds to human CD122 protein with a binding affinity as measured by K_{D} of between about 3E⁻⁰⁹ to 7E⁻¹⁰ M.

Aspect 41: The antibody of any one of aspects 1-28, wherein the antibody binds to human CD122 protein with a binding affinity as measured by K_{D} of between about 2E⁻⁰⁹ to 8E⁻¹⁰ M.

Aspect 42: The antibody of any one of aspects 1-28, wherein the antibody binds to human CD122 protein with a binding affinity as measured by K_{D} of between about 1E⁻⁰⁹ to 9E⁻¹⁰ M.

Aspect 43: The antibody of any one of aspects 1-42, wherein the antibody comprises a heavy chain comprising the sequence of SEQ ID NO: 134 and a light chain comprising the sequence of SEQ ID NO: 142.

Aspect 44: The antibody of any one of aspects 1-42, wherein the antibody comprises a heavy chain comprising the sequence of SEQ ID NO: 135 and a light chain comprising the sequence of SEQ ID NO: 143.

Aspect 45: The antibody of any one of aspects 1-42, wherein the antibody comprises a heavy chain comprising the sequence of SEQ ID NO: 135 and a light chain comprising the sequence of SEQ ID NO: 144.

Aspect 46: The antibody of any one of aspects 1-42, wherein the antibody comprises a heavy chain comprising the sequence of SEQ ID NO: 135 and a light chain comprising the sequence of SEQ ID NO: 145.

Aspect 47: The antibody of any one of aspects 1-42, wherein the antibody comprises a heavy chain comprising the sequence of SEQ ID NO: 136 and a light chain comprising the sequence of SEQ ID NO: 143.

Aspect 48: The antibody of any one of aspects 1-42, wherein the antibody comprises a heavy chain comprising the sequence of SEQ ID NO: 136 and a light chain comprising the sequence of SEQ ID NO: 144.

Aspect 49: The antibody of any one of aspects 1-42, wherein the antibody comprises a heavy chain comprising the sequence of SEQ ID NO: 136 and a light chain comprising the sequence of SEQ ID NO: 145.

Aspect 50: The antibody of any one of aspects 1-42, wherein the antibody comprises a heavy chain comprising the sequence of SEQ ID NO: 137 and a light chain comprising the sequence of SEQ ID NO: 143.

Aspect 51: The antibody of any one of aspects 1-42, wherein the antibody comprises a heavy chain comprising the sequence of SEQ ID NO: 137 and a light chain comprising the sequence of SEQ ID NO: 144.

Aspect 52: The antibody of any one of aspects 1-42, wherein the antibody comprises a heavy chain comprising the sequence of SEQ ID NO: 137 and a light chain comprising the sequence of SEQ ID NO: 145.

Aspect 53: The antibody of any one of aspects 1-42, wherein the antibody comprises a heavy chain comprising the sequence of SEQ ID NO: 138 and a light chain comprising the sequence of SEQ ID NO: 142.

Aspect 54: The antibody of any one of aspects 1-42, wherein the antibody comprises a heavy chain comprising the sequence of SEQ ID NO: 139 and a light chain comprising the sequence of SEQ ID NO: 143.

Aspect 55: The antibody of any one of aspects 1-42, wherein the antibody comprises a heavy chain comprising the sequence of SEQ ID NO: 139 and a light chain comprising the sequence of SEQ ID NO: 144.

Aspect 56: The antibody of any one of aspects 1-42, wherein the antibody comprises a heavy chain comprising the sequence of SEQ ID NO: 139 and a light chain comprising the sequence of SEQ ID NO: 145.

Aspect 57: The antibody of any one of aspects 1-42, wherein the antibody comprises a heavy chain comprising the sequence of SEQ ID NO: 140 and a light chain comprising the sequence of SEQ ID NO: 143.

Aspect 58: The antibody of any one of aspects 1-42, wherein the antibody comprises a heavy chain comprising the sequence of SEQ ID NO: 140 and a light chain comprising the sequence of SEQ ID NO: 144.

Aspect 59: The antibody of any one of aspects 1-42, wherein the antibody comprises a heavy chain comprising the sequence of SEQ ID NO: 140 and a light chain comprising the sequence of SEQ ID NO: 145.

Aspect 60: The antibody of any one of aspects 1-42, wherein the antibody comprises a heavy chain comprising the sequence of SEQ ID NO: 141 and a light chain comprising the sequence of SEQ ID NO: 143.

Aspect 61: The antibody of any one of aspects 1-42, wherein the antibody comprises a heavy chain comprising the sequence of SEQ ID NO: 141 and a light chain comprising the sequence of SEQ ID NO: 144.

Aspect 62: The antibody of any one of aspects 1-42, wherein the antibody comprises a heavy chain comprising the sequence of SEQ ID NO: 141 and a light chain comprising the sequence of SEQ ID NO: 145.

Aspect 63: An isolated monoclonal antibody, wherein, when bound to CD122, the monoclonal antibody binds to at least one of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 64: The isolated monoclonal antibody of aspect 63, wherein, when bound to CD122, the monoclonal antibody binds to at least two of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 65: The isolated monoclonal antibody of aspect 63 or 64, wherein, when bound to CD122, the monoclonal antibody binds to at least three of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 66: The isolated monoclonal antibody of any one of aspects 63-65, wherein, when bound to CD122, the monoclonal antibody binds to at least four of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 67: The isolated monoclonal antibody of any one of aspects 63-66, wherein, when bound to CD122, the monoclonal antibody binds to at least five of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 68: The isolated monoclonal antibody of any one of aspects 63-67, wherein, when bound to CD122, the monoclonal antibody binds to at least six of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 69: The isolated monoclonal antibody of any one of aspects 63-68, wherein, when bound to CD122, the monoclonal antibody binds to at least seven of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 70: The isolated monoclonal antibody of any one of aspects 63-69, wherein, when bound to CD122, the monoclonal antibody binds to at least eight of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 71: The isolated monoclonal antibody of any one of aspects 63-70, wherein, when bound to CD122, the monoclonal antibody binds to at least nine of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 72: The isolated monoclonal antibody of any one of aspects 63-71, wherein, when bound to CD122, the monoclonal antibody binds to at least ten of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 73: The isolated monoclonal antibody of any one of aspects 63-72, wherein, when bound to CD122, the monoclonal antibody binds to at least eleven of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 74: The isolated monoclonal antibody of any one of aspects 63-73, wherein, when bound to CD122, the monoclonal antibody binds to at least twelve of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 75: The isolated monoclonal antibody of any one of aspects 63-74, wherein, when bound to CD122, the monoclonal antibody binds to at least thirteen of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 76: The isolated monoclonal antibody of any one of aspects 63-75, wherein, when bound to CD122, the monoclonal antibody binds to at least fourteen of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 77: The isolated monoclonal antibody of any one of aspects 63-76, wherein, when bound to CD122, the monoclonal antibody binds to at least fifteen of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 78: The isolated monoclonal antibody of any one of aspects 63-77, wherein, when bound to CD122, the monoclonal antibody binds to at least sixteen of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 79: The isolated monoclonal antibody of any one of aspects 63-78, wherein, when bound to CD122, the monoclonal antibody binds to at least seventeen of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 80: The isolated monoclonal antibody of any one of aspects 63-79, wherein, when bound to CD122, the monoclonal antibody binds to at least eighteen of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 81: The isolated monoclonal antibody of any one of aspects 63-80, wherein, when bound to CD122, the monoclonal antibody binds to at least nineteen of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 82: The isolated monoclonal antibody of any one of aspects 63-81, wherein, when bound to CD122, the monoclonal antibody binds to at least twenty of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 83: The isolated monoclonal antibody of any one of aspects 63-82, wherein, when bound to CD122, the monoclonal antibody binds to at least twenty-one of the following residues: W39, P40, D41, V76, D77, 178, V79, T80, L81, F99, K100, P101, F102, E103, N104, L105, H134, Y135, F136, E137, R138, or H139 of SEQ ID NO: 187.

Aspect 84: The isolated monoclonal antibody of any one of aspects 63-83, wherein, when bound to CD122, the monoclonal antibody binds to at least all twenty-two of the following residues: W39, P40, D41, V76, D77, 178, V79, T80, L81, F99, K100, P101, F102, E103, N104, L105, H134, Y135, F136, E137, R138, and H139 of SEQ ID NO: 187.

Aspect 85: The isolated monoclonal antibody of any one of aspects 63-84, wherein the monoclonal antibody binds to at least W39.

Aspect 86: The isolated monoclonal antibody of any one of aspects 63-85, wherein the monoclonal antibody binds to at least P40.

Aspect 87: The isolated monoclonal antibody of any one of aspects 63-86, wherein the monoclonal antibody binds to at least D41.

Aspect 88: The isolated monoclonal antibody of any one of aspects 63-87, wherein the monoclonal antibody binds to at least V76.

Aspect 89: The isolated monoclonal antibody of any one of aspects 63-88, wherein the monoclonal antibody binds to at least D77.

Aspect 90: The isolated monoclonal antibody of any one of aspects 63-89, wherein the monoclonal antibody binds to at least 178.

Aspect 91: The isolated monoclonal antibody of any one of aspects 63-90, wherein the monoclonal antibody binds to at least V79.

Aspect 92: The isolated monoclonal antibody of any one of aspects 63-91, wherein the monoclonal antibody binds to at least T80.

Aspect 93: The isolated monoclonal antibody of any one of aspects 63-92, wherein the monoclonal antibody binds to at least L81.

Aspect 94: The isolated monoclonal antibody of any one of aspects 63-93, wherein the monoclonal antibody binds to at least F99.

Aspect 95: The isolated monoclonal antibody of any one of aspects 63-94, wherein the monoclonal antibody binds to at least K100.

Aspect 96: The isolated monoclonal antibody of any one of aspects 63-95, wherein the monoclonal antibody binds to at least P101.

Aspect 97: The isolated monoclonal antibody of any one of aspects 63-96, wherein the monoclonal antibody binds to at least F102.

Aspect 98: The isolated monoclonal antibody of any one of aspects 63-97, wherein the monoclonal antibody binds to at least E103.

Aspect 99: The isolated monoclonal antibody of any one of aspects 63-98, wherein the monoclonal antibody binds to at least N104.

Aspect 100: The isolated monoclonal antibody of any one of aspects 63-99, wherein the monoclonal antibody binds to at least L105.

Aspect 101: The isolated monoclonal antibody of any one of aspects 63-100, wherein the monoclonal antibody binds to at least H134.

Aspect 102: The isolated monoclonal antibody of any one of aspects 63-101, wherein the monoclonal antibody binds to at least Y135.

Aspect 103: The isolated monoclonal antibody of any one of aspects 63-102, wherein the monoclonal antibody binds to at least F136.

Aspect 104: The isolated monoclonal antibody of any one of aspects 63-103, wherein the monoclonal antibody binds to at least E137.

Aspect 105: The isolated monoclonal antibody of any one of aspects 63-104, wherein the monoclonal antibody binds to at least R138.

Aspect 106: The isolated monoclonal antibody of any one of aspects 63-105, wherein the monoclonal antibody binds to at least H139.

Aspect 107: The isolated monoclonal antibody of any one of aspects 63-106, wherein the monoclonal antibody blocks binding of IL2 to CD122.

Aspect 108: The isolated monoclonal antibody of any one of aspects 63-107, wherein the monoclonal antibody blocks binding of IL15 to CD122.

Aspect 109: The isolated monoclonal antibody of any one of aspects 63-108, wherein the monoclonal antibody blocks binding of IL2 and IL15 to CD122.

Aspect 110: The isolated monoclonal antibody of any one of aspects 63-109, wherein the monoclonal antibody blocks binding of IL2 to an intermediate affinity IL-βγ receptor complex.

Aspect 111: The isolated monoclonal antibody of any one of aspects 63-110, wherein the monoclonal antibody blocks binding of an IL15/IL15Ru complex to an intermediate affinity IL-βγ receptor complex.

Aspect 112: The isolated monoclonal antibody of any one of aspects 63-111, wherein the monoclonal antibody blocks binding of IL2 and an IL15/IL15Ru complex to an intermediate affinity IL-βγ receptor complex.

Aspect 113: The isolated monoclonal antibody of any one of aspects 63-112, wherein the monoclonal antibody is a human antibody.

Aspect 114: The isolated monoclonal antibody of any one of aspects 63-113, wherein the monoclonal antibody is a humanized antibody.

Aspect 115: The isolated monoclonal antibody of any one of aspects 63-114, wherein the isolated monoclonal antibody binds to CD122 with a K_{D} of less than or equal to about 2E⁻⁰⁹ M.

Aspect 116: The isolated monoclonal antibody of any one of aspects 63-115, wherein the isolated monoclonal antibody binds to CD122 with a K_{D} of less than or equal to about 1.5×E⁻⁰⁹ M.

Aspect 117: The isolated monoclonal antibody of any one of aspects 63-116, wherein the isolated monoclonal antibody binds to CD122 with a K_{D} of less than or equal to about 1×E⁻⁰⁹ M.

Aspect 118: The isolated monoclonal antibody of any one of aspects 63-117, wherein the isolated monoclonal antibody binds to CD122 with a K_{D} of less than or equal to about 9.5×E⁻¹⁰ M.

Aspect 119: The isolated monoclonal antibody of any one of aspects 63-118, wherein the isolated monoclonal antibody binds to CD122 with a K_{D} of less than or equal to about 9×E⁻¹⁰ M.

Aspect 120: The isolated monoclonal antibody of any one of aspects 63-119, wherein the isolated monoclonal antibody binds to CD122 with a K_{D} of less than or equal to about 8×E⁻¹⁰ M.

Aspect 121: The isolated monoclonal antibody of any one of aspects 63-120, wherein the isolated monoclonal antibody binds to CD122 with a K_{D} of less than or equal to about 7×E⁻¹⁰ M.

Aspect 122: The isolated monoclonal antibody of any one of aspects 63-121, wherein at least one of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 123: The isolated monoclonal antibody of any one of aspects 63-122, wherein at least two of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 124: The isolated monoclonal antibody of any one of aspects 63-123, wherein at least three of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 125: The isolated monoclonal antibody of any one of aspects 63-124, wherein at least four of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 126: The isolated monoclonal antibody of any one of aspects 63-125, wherein at least five of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 127: The isolated monoclonal antibody of any one of aspects 63-126, wherein at least six of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 128: The isolated monoclonal antibody of any one of aspects 63-127, wherein at least seven of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 129: The isolated monoclonal antibody of any one of aspects 63-128, wherein at least eight of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 130: The isolated monoclonal antibody of any one of aspects 63-129, wherein at least nine of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 131: The isolated monoclonal antibody of any one of aspects 63-130, wherein at least ten of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 132: The isolated monoclonal antibody of any one of aspects 63-131, wherein at least eleven of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 133: The isolated monoclonal antibody of any one of aspects 63-132, wherein at least twelve of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 134: The isolated monoclonal antibody of any one of aspects 63-133, wherein at least thirteen of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 135: The isolated monoclonal antibody of any one of aspects 63-134, wherein at least fourteen of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 136: The isolated monoclonal antibody of any one of aspects 63-135, wherein at least fifteen of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 137: The isolated monoclonal antibody of any one of aspects 63-136, wherein at least sixteen of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 138: The isolated monoclonal antibody of any one of aspects 63-137, wherein at least seventeen of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 139: The isolated monoclonal antibody of any one of aspects 63-138, wherein at least eighteen of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 140: The isolated monoclonal antibody of any one of aspects 63-139, wherein at least nineteen of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 141: The isolated monoclonal antibody of any one of aspects 63-140, wherein at least twenty of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 142: The isolated monoclonal antibody of any one of aspects 63-141, wherein at least twenty-one of residues W39, P40, D41, V76, D77, 178, V79, T80, L81, F99, K100, P101, F102, E103, N104, L105, H134, Y135, F136, E137, R138, or H139 of SEQ ID NO: 187 comprise an epitope on an extracellular domain of CD122.

Aspect 143: The isolated monoclonal antibody of any one of aspects 63-142, wherein at least all twenty-two of residues W39, P40, D41, V76, D77, 178, V79, T80, L81, F99, K100, P101, F102, E103, N104, L105, H134, Y135, F136, E137, R138, and H139 of SEQ ID NO: 187 comprise an epitope on an extracellular domain of CD122.

Aspect 144: The isolated monoclonal antibody of any one of aspects 122-143, wherein the epitope is a functional epitope.

Aspect 145: The isolated monoclonal antibody of any one of aspects 122-143, wherein the epitope is a structural epitope.

Aspect 146: The isolated monoclonal antibody of any one of aspects 122-145, wherein the epitope is an epitope on a native CD122 protein.

Aspect 147: The isolated monoclonal antibody of any one of aspects 63-145, wherein the isolated monoclonal inhibits:
i) binding of IL2 to a high affinity IL-αβγ receptor comprising CD122, CD132, and CD25; or
ii) binding of IL15, presented in trans bound to IL15Rα, to the intermediate affinity IL-βγ receptor comprising CD122 and CD132; or
iii) binding of IL15 to a high affinity IL-αβγ receptor comprising CD122, CD132 and IL15Rα.

What is claimed is:

1. An anti-CD122 antibody comprising i) a heavy chain comprising a variable heavy chain (VH) domain and ii) a light chain comprising a variable light chain (VL) domain, wherein the VH domain comprises an HCDR1 sequence comprising a sequence selected from SEQ ID NOs: 1-11, an HCDR2 sequence comprising a sequence selected from SEQ ID NOs: 12-23, and an HCDR3 sequence comprising a sequence selected from SEQ ID NOs: 24-36, and VL domain comprises an LCDR1 sequence comprising a sequence selected from SEQ ID NOs: 37-47, an LCDR2 sequence comprising a sequence selected from GTS, TTS, YTS, WAS, KAS, GAT, YAS or STS, and an LCDR3 sequence comprising a sequence selected from SEQ ID NOs: 56-67.

2. The anti-CD122 antibody of claim 1, wherein the HCDR1 sequence comprises SEQ ID NO: 1, the HCDR2 sequence comprises SEQ ID NO: 12, the HCDR3 sequence comprises SEQ ID NO: 24, the LCDR1 sequence comprises SEQ ID NO: 37, the LCDR2 sequence comprises SEQ ID NO: GTS, and the LCDR3 sequence comprises SEQ ID NO: 56.

3. The anti-CD122 antibody of claim 1, wherein the HCDR1 sequence comprises SEQ ID NO: 2, the HCDR2 sequence comprises SEQ ID NO: 13, the HCDR3 sequence comprises SEQ ID NO: 25, the LCDR1 sequence comprises SEQ ID NO: 38, the LCDR2 sequence comprises SEQ ID NO: TTS, and the LCDR3 sequence comprises SEQ ID NO: 57.

4. The anti-CD122 antibody of claim 1, wherein the HCDR1 sequence comprises SEQ ID NO: 3, the HCDR2 sequence comprises SEQ ID NO: 14, the HCDR3 sequence comprises SEQ ID NO: 26, the LCDR1 sequence comprises SEQ ID NO: 39, the LCDR2 sequence comprises SEQ ID NO: YTS, and the LCDR3 sequence comprises SEQ ID NO: 58.

5. The anti-CD122 antibody of claim 1, wherein the HCDR1 sequence comprises SEQ ID NO: 4, the HCDR2 sequence comprises SEQ ID NO: 15, the HCDR3 sequence comprises SEQ ID NO: 27, the LCDR1 sequence comprises SEQ ID NO: 40, the LCDR2 sequence comprises SEQ ID NO: WAS, and the LCDR3 sequence comprises SEQ ID NO: 59.

6. The anti-CD122 antibody of claim 1, wherein the HCDR1 sequence comprises SEQ ID NO: 5, the HCDR2 sequence comprises SEQ ID NO: 16, the HCDR3 sequence comprises SEQ ID NO: 28, the LCDR1 sequence comprises SEQ ID NO: 41, the LCDR2 sequence comprises SEQ ID NO: YTS, and the LCDR3 sequence comprises SEQ ID NO: 60.

7. The anti-CD122 antibody of claim 1, wherein the HCDR1 sequence comprises SEQ ID NO: 6, the HCDR2 sequence comprises SEQ ID NO: 17, the HCDR3 sequence comprises SEQ ID NO: 29, the LCDR1 sequence comprises SEQ ID NO: 42, the LCDR2 sequence comprises SEQ ID NO: KAS, and the LCDR3 sequence comprises SEQ ID NO: 61.

8. The anti-CD122 antibody of claim 1, wherein the HCDR1 sequence comprises SEQ ID NO: 7, the HCDR2 sequence comprises SEQ ID NO: 18, the HCDR3 sequence comprises SEQ ID NO: 30, the LCDR1 sequence comprises SEQ ID NO: 43, the LCDR2 sequence comprises SEQ ID NO: YTS, and the LCDR3 sequence comprises SEQ ID NO: 62.

9. The anti-CD122 antibody of claim 1, wherein the HCDR1 sequence comprises SEQ ID NO: 8, the HCDR2 sequence comprises SEQ ID NO: 19, the HCDR3 sequence comprises SEQ ID NO: 31, the LCDR1 sequence comprises SEQ ID NO: 44, the LCDR2 sequence comprises SEQ ID NO: YTS, and the LCDR3 sequence comprises SEQ ID NO: 63.

10. The anti-CD122 antibody of claim 1, wherein the HCDR1 sequence comprises SEQ ID NO: 9, the HCDR2 sequence comprises SEQ ID NO: 20, the HCDR3 sequence comprises SEQ ID NO: 32, the LCDR1 sequence comprises SEQ ID NO: 45, the LCDR2 sequence comprises SEQ ID NO: GAT, and the LCDR3 sequence comprises SEQ ID NO: 64.

11. The anti-CD122 antibody of claim 1, wherein the HCDR1 sequence comprises SEQ ID NO: 1, the HCDR2 sequence comprises SEQ ID NO: 21, the HCDR3 sequence comprises SEQ ID NO: 33, the LCDR1 sequence comprises SEQ ID NO: 37, the LCDR2 sequence comprises SEQ ID NO: GTS, and the LCDR3 sequence comprises SEQ ID NO: 65.

12. The anti-CD122 antibody of claim 1, wherein the HCDR1 sequence comprises SEQ ID NO: 1, the HCDR2 sequence comprises SEQ ID NO: 21, the HCDR3 sequence comprises SEQ ID NO: 34, the LCDR1 sequence comprises SEQ ID NO: 37, the LCDR2 sequence comprises SEQ ID NO: GTS, and the LCDR3 sequence comprises SEQ ID NO: 65.

13. The anti-CD122 antibody of claim 1, wherein the HCDR1 sequence comprises SEQ ID NO: 10, the HCDR2 sequence comprises SEQ ID NO: 22, the HCDR3 sequence comprises SEQ ID NO: 35, the LCDR1 sequence comprises SEQ ID NO: 46, the LCDR2 sequence comprises SEQ ID NO: YAS, and the LCDR3 sequence comprises SEQ ID NO: 66.

14. The anti-CD122 antibody of claim 1, wherein the HCDR1 sequence comprises SEQ ID NO: 11, the HCDR2 sequence comprises SEQ ID NO: 23, the HCDR3 sequence comprises SEQ ID NO: 36, the LCDR1 sequence comprises SEQ ID NO: 47, the LCDR2 sequence comprises SEQ ID NO: STS, and the LCDR3 sequence comprises SEQ ID NO: 67.

15. The anti-CD122 antibody of claim 1, wherein the VH domain comprises at least 80%, at least 85%, at least 90%, or at least 95% sequence identity to a sequence selected from SEQ ID NOs: 82-94.

16. The anti-CD122 antibody of claim 1, wherein the VL domain comprises at least 80%, at least 85%, at least 90%, or at least 95% sequence identity to a sequence selected from SEQ ID NOs: 95-107.

17. The anti-CD122 antibody of claim 1, wherein the VH domain is encoded by a nucleic acid comprising at least 80%, at least 85%, at least 90%, or at least 95% sequence identity to a sequence selected from SEQ ID NOs: 108-120.

18. The anti-CD122 antibody of claim 1, wherein the VL domain is encoded by a nucleic acid comprising at least 80%, at least 85%, at least 90%, or at least 95% sequence identity to a sequence selected from SEQ ID NOs: 121-133.

19. The anti-CD122 antibody of claim 1, wherein the heavy chain comprises at least 80%, at least 85%, at least 90%, or at least 95% sequence identity to a sequence selected from SEQ ID NOs: 134-141.

20. The anti-CD122 antibody of claim 1, wherein the light chain comprises at least 80%, at least 85%, at least 90%, or at least 95% sequence identity to a sequence selected from SEQ ID NOs: 142-145.

21. The anti-CD122 antibody of claim 1, wherein the heavy chain is encoded by a nucleic acid comprising at least 80%, at least 85%, at least 90%, or at least 95% sequence identity to a sequence selected from SEQ ID NOs: 146-153.

22. The anti-CD122 antibody of claim 1, wherein the light chain is encoded by a nucleic acid comprising at least 80%, at least 85%, at least 90%, or at least 95% sequence identity to a sequence selected from SEQ ID NOs: 154-157.

23. The anti-CD122 antibody of claim 1, wherein the heavy chain comprises a leader sequence at an N-terminus of a heavy chain polypeptide.

24. The anti-CD122 antibody of claim 23, wherein the leader sequence comprises SEQ ID NO: 158, SEQ ID NO: 159, SEQ ID NO: 160, or SEQ ID NO: 161.

25. The anti-CD122 antibody of claim 1, wherein the light chain comprises a leader sequence at an N-terminus of a light chain polypeptide.

26. The anti-CD122 antibody of claim 25, wherein the leader sequence comprises SEQ ID NO: 158, SEQ ID NO: 159, SEQ ID NO: 160, or SEQ ID NO: 161.

27. The anti-CD122 antibody of any one of claims 1-26, wherein the anti-CD122 antibody is a humanized antibody or antigen binding fragment thereof.

28. The anti-CD122 antibody of any one of claims 1-27, wherein the anti-CD122 antibody is a chimeric antibody or antigen binding fragment thereof.

29. The anti-CD122 antibody of any one of claims 1-28, wherein the anti-CD122 antibody comprises an IgG-scFv, nanobody, mini-antibody, minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, or intrabody.

30. The anti-CD122 antibody of any one of claims 1-29, wherein the anti-CD122 antibody is an IgG1 antibody.

31. The anti-CD122 antibody of any one of claims 1-30, wherein the anti-CD122 antibody is an IgG2 antibody.

32. The anti-CD122 antibody of any one of claims 1-31, wherein the anti-CD122 antibody is an IgG4 antibody.

33. The anti-CD122 antibody of any one of claims 1-32, wherein the light chain is a kappa chain.

34. The anti-CD122 antibody of any one of claims 1-33, wherein the anti-CD122 antibody has a binding affinity to human CD122 of about 100 μM to about 3 nM.

35. A pharmaceutical composition comprising an anti-CD122 antibody of any one of claims 1-34 and a pharmaceutically acceptable excipient.
